(19) United States
(12) Patent Application Publication
(10) Pub. No.: US 2026/0010781 A1
(43) Pub. Date: Jan. 8, 2026

(54) SYSTEMS AND METHODS FOR MODELING AND DECODING NEURAL ACTIVITIES

(71) Applicants: The Trustees of Columbia University in the City of New York, New York, NY (US); The Regents of The University of California, Oakland, CA (US)

(72) Inventors: Vivek ATHALYE, New York, NY (US); Rui COSTA, New York, NY (US); Jose CARMENA, Berkeley, CA (US); Preeya KHANNA, San Francisco, CA (US)

(21) Appl. No.: 19/330,303

(22) Filed: Sep. 16, 2025

Related U.S. Application Data
(63) Continuation of application No. PCT/US2024/020092, filed on Mar. 15, 2024.
(60) Provisional application No. 63/491,028, filed on Mar. 17, 2023.

(51) Int. Cl. G06N 3/06; A61B 5/00; G06F 3/01

(52) U.S. Cl. CPC G06N 3/061; A61B 5/7228; A61B 5/7278; A61B 5/7282; G06F 3/015

(57) ABSTRACT

Methods and systems are disclosed for modeling and decoding neural activities. A brain machine interface (BMI) measures neural activities. A processor receives signals corresponding to the neural activities from the BMI. The processor generates by applying a neural dynamics model signals corresponding to a de-noised state of the neural activities. By applying a BMI decoding model to the de-noised signals, the processor generates a control signal, which is used by a BMI plant model to generate a movement vector. An object is moved according to a predetermined path, in response to the movement vector and the current state of the object. In response to the object's movement, the BMI continuously measures the neural activities. Coefficients of the neural dynamics model and the BMI decoding model are iteratively updated, by executing a learning algorithm, in response to the BMI's continuous measurement.

### CROSS-REFERENCE TO RELATED APPLICATION

This application is a continuation of International Patent Application No. PCT/US2024/020092, filed on Mar. 15, 2024, which claims the benefit of priority to U.S. Provisional Application No. 63/491,028, entitled “Systems and Methods for Modeling and Decoding Neural Activities,” filed on Mar. 17, 2023, the disclosures of which are hereby incorporated by reference in their entirety.

All patents, patent applications and publications cited herein are hereby incorporated by reference in their entirety. The disclosures of these publications in their entireties are hereby explicitly incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT INFORMATION

This invention was made with government support under NS104649 and NS128250 awarded by the National Institutes of Health. The government has certain rights in the invention.

### TECHNICAL FIELD

The present disclosure relates generally to brain-machine interfaces (BMIs) and dynamics of neural population activity.

### BACKGROUND

BMIs (sometimes also referred to as brain-computer interfaces, or BCIs) can be applied in many areas involving problems facing humans, such as restoring lost motor function to people suffering such neurological injury or disease. Moreover, BMIs provide new mechanisms for controlling a wide variety of machines and/or computing devices by translating neural activities to commands. For example, a BMI can be used to allow a paralyzed human to control a prosthetic device such as an arm, a leg, a hand, a foot, a finger, a toe, etc. Likewise, a BMI can enable monitoring of the brain to detect a state of the brain, such as a seizure state, a resting state, an awake state, etc.

BMI technology can use a decoder to decode a user's intentions directly from brain signals, for example, collected by implanted electrodes. BMI systems and techniques can involve Kalman filter techniques, population vector techniques, and external kinematic parameters, such as arm velocity, cursor position, and cursor velocity.

### SUMMARY

Methods and systems are disclosed for modeling and decoding neural activities. In some embodiments, a brain machine interface (BMI) measures neural activities. A processor receives signals corresponding to the neural activities from the BMI. The processor generates by applying a neural dynamics model signals corresponding to a de-noised state of the neural activities. By applying a BMI decoding model to the de-noised signals, the processor generates a control signal, which is used by a BMI plant model to generate a movement vector. An object is moved according to a predetermined path, in response to the movement vector and the current state of the object. In response to the object's movement, the BMI continuously measures the neural activities. Coefficients of the neural dynamics model and the BMI decoding model are iteratively updated, by executing a learning algorithm, in response to the BMI's continuous measurement.

In accordance with some embodiments, mechanisms (which can include systems, methods, and media) for brain-machine interfaces (BMIs) are provided. As used herein, a BMI is a device that can interface with a nervous system (which can include the brain, the spinal cord, and peripheral nerves) and/or with muscles in order to translate neural information into one or more signals to be subsequently used for controlling an external device, monitoring the state of the brain, visualizing neural activity, and/or for any other suitable purpose. These mechanisms can be used with human subjects and/or any other suitable animal subjects, in some embodiments. These mechanisms can include receiving neural data of subjects for a period of time using one or more sensors, and transforming the neural data to generate aligned variables.

In some embodiments, a “neuron” as described herein can actually be the combined response of a small number of nearby neurons. Thus, when a “neuron” is referred to herein, the neuron can be whatever is the source of spiking activity that is detected by a single electrode or sensor being used to make observations.

In some embodiments, the activity of one or more neurons can be inferred indirectly from other measurements. For example, electrical activity measured from one or more muscles can be observed. Based on that electrical activity, the activity of motor neurons in the spinal cord can be inferred.

In some embodiments, neural activities can be modeled in a multi-dimensional state space in which each dimension represents the firing rate of a neuron being observed. Neural activity at any given time corresponds to a single location in this state space (the “neural state”), in some embodiments. The “firing rate” of a neuron at a given time is a numerical value related to how many spikes the neuron is likely to emit in a brief window of time. Models can be constructed to determine a latent spatiotemporal representation of one or more neural state variables, which are linked to neuron activities, for the period of time. Systems and methods disclosed by the present application include decoding the latent spatiotemporal representation into a state of a neural population for the period of time.

In some embodiments, systems disclosed by the present application include one or more processors, and one or more memory devices having stored computer-executable instructions. The instructions can be executable by the one or more processors to cause the disclosed system to perform at least receiving neural data for a period of time from one or more sensors, transforming the neural data to aligned variables, and operating with the aligned variables through a model representing spatiotemporal representations of activities of a neural population.

In some embodiments, using systems and methods disclosed by the present application, an operator can interface the brain of human subjects and/or any other suitable animal subjects with a computer via a brain-computer interface (BCI). The disclosed BCI can continuously learn and/or update, without external supervision, a model of the dynamics of brain activity with reference to the user's behavior and context. In addition, the disclosed BCI can decode information from the brain in a manner that generalizes across different behavior and contexts. The disclosed systems and methods can control an object not limited to a computer cursor. The controlled object can be a graphical object displayed on a display device and/or a physical object. In an example, the system decodes how the brain would like to control the movement of a prosthetic such as a computer cursor or prosthetic limb, and other suitable items that can be controlled mechanically and/or electronically, generalizing across different types of movements and contexts. Since the disclosed model does not require supervised training, the disclosed system doesn't require the user to perform instructed behavior in order for the system to learn the brain's dynamics. In another example, the system can learn models of various dynamics without supervised calibration in order to accurately decode how the brain would like to move for both slow and fast prosthetic movements. In another example, the system can learn models of various dynamics, which correspond to the brain exhibiting activities when using different computer applications, without supervised calibration in order to decode information from the brain across different contexts.

In some embodiments, the disclosed system improves the performance of decoding information from the brain across different behaviors and contexts when operated by a user. For example, the system can decode a variety of information (e.g., attention, mood, cognitive load, anesthesia level, hunger, thirst). The system can also encode information into the brain in a manner that generalizes across different contexts of behavior, where information can be encoded through sensory stimulus presentation or through direct stimulation of the brain.

### BRIEF DESCRIPTION OF THE DRAWINGS

FIG. 1 is a simplified diagram illustrating an example system for decoding neural dynamics with a BMI, according to some embodiments.

FIG. 2 is a simplified diagram illustrating an example system for modeling and decoding neural dynamics, according to some embodiments.

FIG. 3 is a simplified diagram illustrating an example system for processing brain activity information with models, according to some embodiments.

FIG. 4 is a flow chart illustrating an example method for modeling and decoding neural dynamics, according to some embodiments.

FIG. 5 is a simplified diagram illustrating an example method for modeling and decoding neural dynamics, according to some embodiments.

FIG. 6 is a flow chart illustrating an example method for modeling and decoding neural dynamics, according to some embodiments.

FIG. 7 is a simplified diagram illustrating an example method for modeling and decoding neural dynamics, according to some embodiments.

FIG. 8 is a simplified diagram illustrating an example system for modeling and decoding neural dynamics, according to some embodiments.

FIG. 9 is an example of hardware processor that can be used in accordance with some embodiments.

FIG. 10 is a simplified diagram illustrating an example system for simulating control mechanisms of neural dynamics, according to some embodiments.

FIGS. 11A-22K illustrate intermediary results of using the disclosed systems and methods, according to some embodiments.

Various objectives, features, and advantages of the disclosed subject matter can be more fully appreciated with reference to the following detailed description of the disclosed subject matter when considered in connection with the following drawings, in which like reference numerals identify like elements.

### DETAILED DESCRIPTION

Systems and methods disclosed in the current application are directed at a subject matter related to BMIs and dynamics of neural population activity, and more specifically, to techniques of modeling and decoding neural dynamics across various behavior and contexts. In some embodiments, a brain-machine interface is operated to use signals from the brain of a subject to control prosthetic devices. For example, the disclosed system can improve techniques to restore lost motor functions of patients suffering from neurological injury or disease. In an example, the disclosed brain-machine interface consists of a sensor to measure the activity of neurons in the brain. It also uses hardware processors to process mathematical models to transform neural activity into the movement of a prosthetic device. Examples of a prosthetic device generally include a robotic limb, a cursor on a computer screen, etc.

In some embodiments, the disclosed systems and methods train models to adjust and/or update their parameters for the operation of a brain-machine interface. Signals are received from the brain of a subject by a brain sensing program. The received signals are processed in a series of transformation operations using a neural dynamics model, a BMI decoding model, a BMI plant model, and a cursor interface program. In other embodiments, an augmented dynamics model is also used in addition to the BMI decoding model. The augmented dynamics model is configured to transform the received signals by receiving inputs from the neural dynamics model and outputting to the BMI plant model.

In some embodiments, the disclosed systems and methods provide mechanisms for a BMI interface to perform well and generalize across different subject behaviors and in various contexts. For example, the disclosed systems and methods use a neural dynamics model in addition to a BMI decoding model and a BMI plant model. As a result, neural activities across different movement and contexts can be more accurately modeled. In an example, the neural dynamics model infers two mechanisms: a state that evolves in time following recurrent dynamics, and an input that drives the state. In another example, a supervised calibration period is implemented by the disclosed systems and methods. The supervised calibration period involves specifying a variety of predetermined target movement trajectories. By using these predetermined target movement trajectories, the disclosed systems and methods can identify a neural dynamics model and a BMI decoding model that accurately model neural activities across different subject behaviors. In yet another example, the neural dynamics model's parameters can be updated by an unsupervised learning method. As a result, the models configured to be updated can accurately model neural activities across various subject behaviors and in various contexts. In a further example, the neural dynamics model comprises parameters that can be switched between modes, based on whether certain mode can more accurately predict neural activities. The BMI with the disclosed systems and methods can operate in different modes for different phases within a movement. It can also operate in different modes when the movement is being controlled in different contexts (e.g., by operating different software applications on a computer, or movement in various settings).

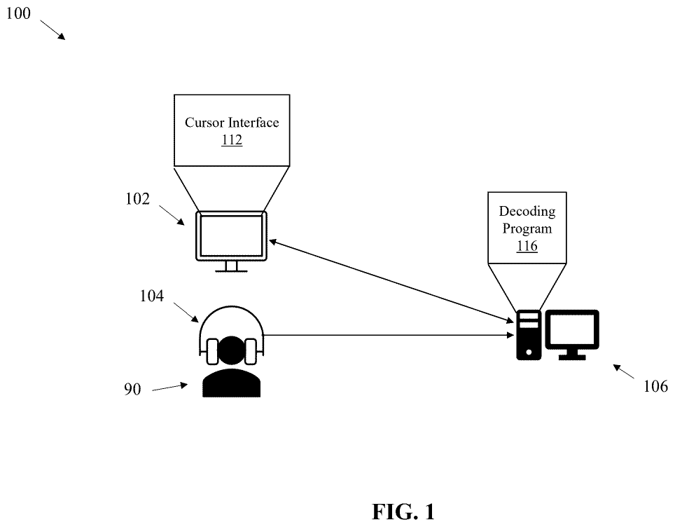

FIG. 1 illustrates an example decoding system 100 for decoding neural activities in the brain of a subject 90, in accordance with some embodiments. The decoding system 100 includes a first computing device 102, a recording device 104, and a second computing device 106. The first computing device 102 is communicatively coupled to the second computing device 106, which is communicatively coupled to the recording device 104. Examples of the computing devices 102 and 106 generally include a computing device or system, such as personal computer, an on-premises server, a cloud-based server, or the like. Examples of communication channels coupling the devices 102, 104, and 106 generally include wired and/or wireless links, and/or a network including one or more local area networks (LANs), wide area networks (WANs), wired networks, wireless networks, the Internet, or the like. In some embodiments, the second computing device 106 is directly connected to and/or integrated within the first computing device 102. In other embodiments, the recording device 104 is directly connected to and/or integrated within the second computing device 106.

The recording device 104 is configured to record neural activities in the brain of the subject 90 and generate a signal corresponding to the recorded neural activities. In turn, the recording device 104 is configured to send the signal corresponding to the recorded neural activities to the second computing device 106, via the communication channel between them. In some embodiments, the recording device 104 comprise chronically implanted microwire electrode arrays spanning bilateral dorsal premotor cortex and primary motor cortex.

In some embodiments, the first computing device 102 includes a processor and a memory. The memory stores instructions and/or data. When executed by the processor, the instructions and/or data stored by the memory causes the processor to run software programs, for example, a cursor interface program 112. By operating the cursor interface program 112 on the first computing device 102, the subject 90 passively watches a cursor, on a display of the first computing device 102, move according to tasks predetermined by the cursor interface program 112. Then, having been trained in advance, the subject 90 controls the cursor to move (e.g., performing a center-out task in which the goal was to move the cursor from the center of the workspace to one of eight radial targets). In turn, the subject 90 performs an obstacle-avoidance task with the same goal plus the constraint of avoiding an obstacle blocking the straight path to the target. During the process of the subject 90 interacting with the display controlled by the cursor interface program 112, the recording device 104 records neural activities in the brain of the subject 90.

Similarly, the second computing device 106 includes a processor and a memory. The memory stores instructions and/or data. When executed by the processor, the instructions and/or data stored by the memory causes the processor to run software programs, for example, a decoding program 116. By operating the decoding program 116 on the second computing device 106, the neural activities in the brain of the subject 90, which are recorded by the recording device 104, are then decoded. For example, the neural activities can be linearly transformed into a two-dimensional command signal, which can be processed in further steps.

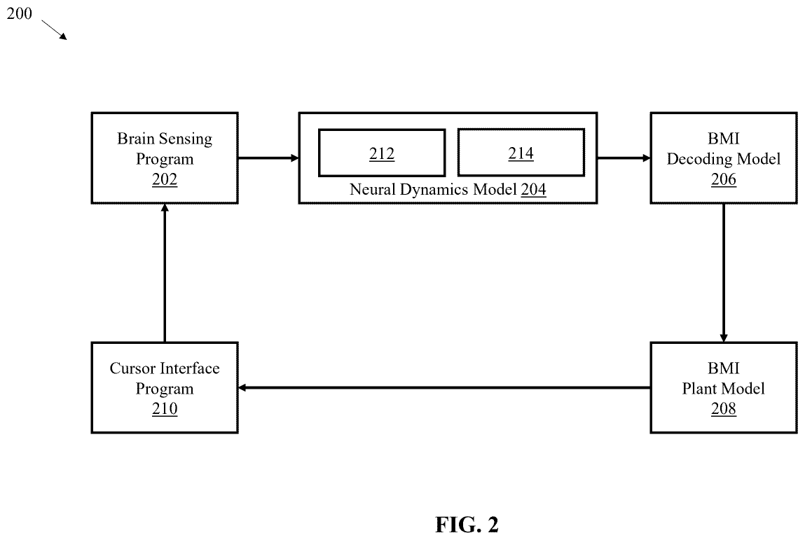

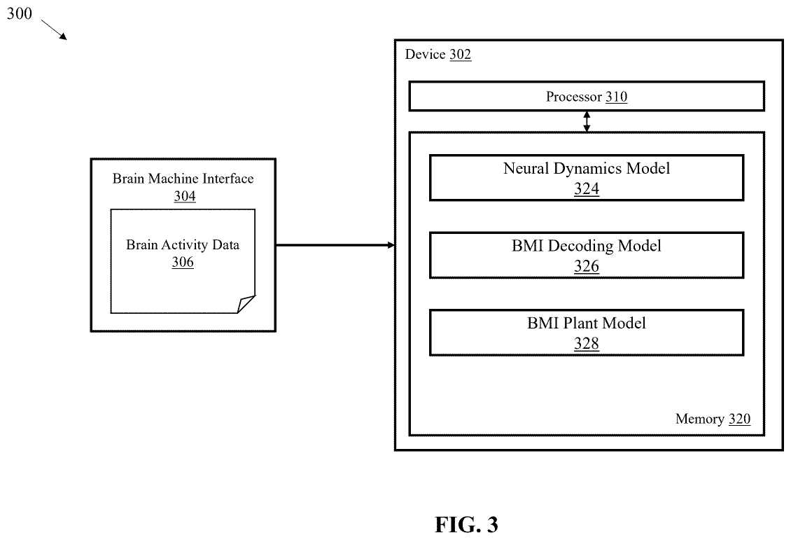

FIG. 2 illustrates an example decoding system 200 for decoding neural activities using a variety of models, in accordance with some embodiments. In the illustrated example, the decoding system 200 includes a brain sensing program 202, a neural dynamics model 204, a BMI decoding model 206, a BMI plant model 208, and a cursor interface program 210. In some embodiments, the programs 202 and 210 as well as models 204, 206, and 208 operate on one or more computing devices (e.g., the computing devices illustrated and described in FIG. 1). More details about models that operate on computing devices are described in below FIG. 3.

The brain sensing program 202 is configured to record neural activities in the brain of a subject and generate a signal corresponding to the recorded neural activities. For example, the brain sensing program 202 can measure neural activities by operating on the recording device 104 illustrated and described in FIG. 1. In turn, the brain sensing program 202 is configured to output the generated signal to the neural dynamics model 204.

In some embodiments, the brain sensing program 202 is configured to operate on a brain sensor. The brain sensor can be an electrode array implanted invasively into motor regions of the brain such as the motor cortex. Examples of brain sensors generally include Electroencephalography (EEG) electrode array, Electrocorticography (ECoG) electrode array, High-density electromyography electrode array (HD-EMG), etc. An electrode records the activity of multiple neurons. By operating brain sensing program 202, the brain sensor can, for example, decompose the measured voltage waveforms into the waveforms of individual neurons (sometimes also referred to as spike sorting). Then, the brain sensing program 202 is configured to count the number of action potentials that each recorded neuron emits in a predetermined unit of time. The operation of counting results in a vector of firing rates. Elements of the vector corresponds to one recorded neuron's firing rate. In this example, the vector of firing rates at a predetermined point of time is recorded to correspond to the neural activity being sensed.

Receiving the signal generated by the brain sensing program 202, which correspond to the measured neural activities, the neural dynamics model 204 is configured to transform the received signal to a neural state signal. In an example, the neural dynamics model 204 receives the measured neural activity from the brain sensing program 202. The neural dynamics model 204 then extracts latent states that represent specific spatiotemporal structure in the neural activity of the subject. Based on at least these latent states, the neural dynamics model 204 is configured to construct a de-noised neural signal. The de-noised neural signal corresponds to a de-noised version of the neural activities being measured. In some embodiments, the neural dynamics model 204 comprise a supervised sub-model 212 and an unsupervised sub-model 214. The supervised sub-model 212 is configured to operate by updating its parameters in response to the user of the disclosed system performing a set of predetermined procedures. The unsupervised sub-model 214 is configured to operate by updating its parameters without interventions from the user of the disclosed system.

In some embodiments, the neural dynamics model 204 makes use of the following mathematical formulation of the dynamics of neural activity:

$${z}_{t + 1} = h ( {z}_{t} , {input}_{t} )$$

$${x}_{t} = g ( {z}_{t} , {input}_{t} )$$

$${y}_{t} = {x}_{t} + {observation\_noise}_{t}$$

yₜ∈R^{N} is the vector of observed activity at time t of N recorded neurons. In an example BMI implementation, this activity is the count of the number of spikes of each neuron in a time bin. observation_noiseₜ∈R^{N} is noise that causes variability in neural activity, which can arise from biological sources such as the unreliability of synaptic transmission between neurons, and can arise from measurement sources such as noise from the electronics that record and pre-process the voltage signals from the brain. This noise corrupts the commands that the brain seeks to send to the BMI. The purpose of modeling the dynamics of neural activity is to remove the effect of this noise on the commands that are decoded from the brain. xₜ∈R^{k} is the de-noised version of the measured neural activity. It can be the signal that the BMI decoding model 206 uses to decode the brain's commands. zₜ∈R^{k} is the underlying latent state at time t of the neural activity, and it can have different dimensionality than the number of recorded neurons. zₜ influences zₜ₊₁. Because neurons have recurrent connections, their present activity influences their future activity. The formulation captures the influence of zₜ on zₜ₊₁ with the dynamics function h. The formulation captures the relationship between latent state zₜ and observed neural activity with the “emissions” function g. inputₜ ∈R^{k} is external input at time t to the recorded neurons, which also drives the evolution in time of latent state zₜ to zₜ₊₁. The physical sources of inputₜ are the unrecorded neurons in the brain that send connection to the recorded neurons, and ultimately the sensory input to the brain. In an example BMI implementation, the neural dynamics model 204 is configured to infer inputₜ, which allows improved estimates of latent state zₜ.

In turn, the neural dynamics model 204 is configured to output generated the neural state signal to the BMI decoding model 206. Receiving the neural state signal, the BMI decoding model 206 is configured to transform the neural state signal to a control signal (sometimes also referred to as a command signal) using a plurality of coefficients of the BMI decoding model 206. The control signal can be used by a processor to linearly update a two-dimensional velocity vector of a computer cursor. For example, the velocity vector of a computer cursor controls the velocity of a cursor to be displayed by the cursor interface program 210. The BMI decoding model 206 is then configured to send the control signal to the BMI plant model 208.

In some embodiments, the BMI decoding model 206 takes a de-noised neural signal xₜ to compute, by linear transformation, the control signal for prosthetic devices. The control signal can be used to update the state of a prosthetic device.

The BMI plant model 208 is configured to combine the control signal generated by the BMI decoding model 206 and cursor state signal corresponding to a current state of the cursor to be displayed by the cursor interface program 210. As a result, the BMI plant model 208 generates a movement signal that determines the velocity and the position of the computer cursor in a next state.

In some embodiments, the state of the prosthetic device (written here as cursorₜ) generally can be updated according to the device's dynamics due to physics of the prosthetic device. For example, a cursor's position can be determined by integrating its velocity. In this example, linear prosthetic dynamics (e.g., position integrating velocity) can be represented with the matrix F:

$${cursor}_{t + 1} = F {cursor}_{t} ( e . g . , updating the cursor based on its own state )$$

$${cursor}_{t + 1} = F {cursor}_{t} + {Kx}_{t} ( e . g . , updating the cursor based on its own state and the neural signal )$$

Where Kxₜ corresponds to the control signal outputted by the BMI decoding model 206.

In other embodiments, non-linear dynamics like the movement of a two-link robotic arm can be linearized and then be transformed as above.

The movement signal is in turn executed by the cursor interface program 210 to display movements of the computer cursor using a display device of a computing device. The displayed cursor movements can be visible to the subject, whose neural activities in the brain are measured by the brain sensing program 202. Viewing cursor movements in the next state stimulates the subject's brain, thereby generating changes to the brain's neural activities. Thus, the neural state signal obtained by measuring the brain's neural activities will update accordingly, which will drive propagations of new signals along the models 204, 206, and 208.

In some embodiments, the cursor interface program 210 is configured to provide feedback to the subject using the BMI about the movement of the prosthetic device (e.g., the computer cursor). For instance, a two-dimensional or three-dimensional cursor is displayed on a display device, and the subject can control the cursor's movement. In some examples, the movement of an avatar is controlled by the subject on the display device. In other examples, the movement of a physical robotic limb is controlled without the subject using the display device. In further examples, the movement of the subject's limb is controlled by the control signal from the BMI decoding model 206 to stimulate the subject's limb muscles.

In some embodiments, the neural dynamics model 204 comprises the following coefficients for the operation and update of the model 204:

$${z}_{t + 1} = {Az}_{t} + {input}_{t}$$

$${x}_{t} = {Cz}_{t}$$

$${y}_{t} = {x}_{t} + {observation\_noise}_{t}$$

yₜ∈R^{N} is the measured neural activity. observation_noiseₜ∈R^{N} is modeled as a multivariate Gaussian distribution with diagonal covariance. xₜ∈R^{N} is the de-noised neural activity, e.g., the prediction of neural activity given the latent state. It is the signal that the BMI decoding model 206 will use. zₜ∈R^{k} is the latent state. zₜ influences zₜ₊₁ through the linear transformation represented with the matrix A (e.g., the recurrent dynamics matrix). Latent state zₜ predicts neural activity yₜ through the linear transformation represented with the emissions matrix C. inputₜ∈R^{k} is modeled as a multivariate Gaussian with diagonal covariance. Thus, inputₜ is modeled as uncorrelated in time and across latent dimensions. This is a minimal model of inputₜ (In the literature in standard formulations of this model, this term is often called noise.) Altogether, this is a latent linear dynamical system which can be optimized using, for example, the EM algorithm.

Dynamics parameters A, C in conjunction with the BMI decoding model 206 parameter are updated as the user operates the BMI during a supervised calibration period. The calibration period is termed “supervised” because the subject uses the BMI to perform specific movement trajectories that are hypothesized to be enable excellent joint fits of the dynamics model 204 parameters and the BMI decoding model 206 parameters.

After the supervised calibration period, the dynamics parameters A, C are continuously updated by the operation of the neural dynamics model 204 as the user operates the BMI. This allows updates to the dynamics in case: 1) the dynamics parameters change because the user's brain exhibits learning, and/or 2) the user performs new movements with the BMI that engage new dynamics that were not observed during the calibration period.

The disclosed linear dynamics model allows A, C to be updated in an unsupervised manner, even as the user operates the BMI. Concretely, A, C are optimized to predict neural activity at time t (yₜ) given the measurement of previous neural activity. This training data is being recorded by the BMI as the user operates the BMI, permitting A, C to be continuously updated. Since A, C is updated so that xₜ predicts yₜ, continuously updating A, C does not negatively impact the BMI decoder model that uses xₜ as input in order to control cursor movements by the operation of the BMI plant model 208.

In other embodiments, the neural dynamics model 204 comprises the following coefficients for the operation and update of the model 204:

$${z}_{t + 1} = {Az}_{t} + B {input}_{t}$$

$${x}_{t} = C {z}_{t} + D {input}_{t}$$

$${y}_{t} = {x}_{t} + {observation\_noise}_{t}$$

In this example, the neural dynamics model 204 separates activity subspaces for input and those for recurrent dynamics. It performs better inference of inputₜ by making use of the following property: neural activity modulates in different dimensions when it is driven by input than the dimensions in which it follows recurrent dynamics. The neural activity dimensions modulated by input are encoded in the matrix D in the model. The neural activity dimensions where activity follows recurrent dynamics are encoded in the matrix C in the model.

yₜ∈R^{N} is the measured neural activity. observation_noiseₜ∈R^{N} is modeled as a multivariate Gaussian distribution with diagonal covariance. xₜ∈R^{N} is the de-noised neural activity, e.g., the prediction of neural activity given the latent state zₜ and inputₜ It is the signal that the BMI decoding model 206 will use. zₜ∈R^{k}^{₁} is the latent state. zₜ influences zₜ₊₁ through the linear transformation represented with the matrix A (the recurrent dynamics matrix). It predicts neural activity yₜ through the linear transformation represented with the emissions matrix C. In some examples, zₜ occupies the dimensions of neural activity spanned by the columns of C. inputₜ∈R^{k}^{₂} is modeled as a multivariate Gaussian with identity covariance. It drives the latent state through the linear transformation represented by the input matrix B. It predicts neural activity yₜ through the linear transformation represented with the emissions matrix D. In some examples, inputₜ occupies the dimensions of neural activity spanned by the columns of D. To utilize the observation that input-driven neural activity modulates in dimensions different from activity following recurrent dynamics, in the supervised calibration period, the user of the disclosed system can perturb the movement of the BMI plant in order collect data in which external input drives the neural activity to make a corrective movement. This data helps estimating the particular parameters B, D. In one implementation of the BMI, parameters B, D can be fixed after supervised calibration, and the remaining parameters A, C can be continuously adapted.

In further embodiments, the neural dynamics model 204 comprises the following coefficients for the operation and update of the model 204:

$${z}_{t + 1} = {Az}_{t} + {B}_{mode} {input}_{t}$$

$${x}_{t} = C {z}_{t} + {D}_{mode} {input}_{t}$$

$${y}_{t} = {x}_{t} + {observation\_noise}_{t}$$

In this example, the neural dynamics model 204 switches between discrete modes of inputs while maintaining invariant recurrent dynamics. The discrete modes of inputs are used to drive the recurrent dynamics. Specifically, the model of the recurrent dynamics stay invariant, but the model of inputs driving the dynamics switches in different discrete modes. The subject can enter discrete modes of movement control, for example, planning to move while withholding movement, initiating movement from quiescence, controlling an ongoing movement that is proceeding as desired, and correcting a large error based on movement feedback. Modes can also include different tasks and/or classes of movements being performed. The neural dynamics model 204 can infer what discrete mode of movement control is currently happening, and switches the model of inputs to the recurrent dynamics. Modes can be inferred from neural activity, muscle activity, behavior, other biometrics such as heart rate, pupil diameter and the like.

B_{mode}−inputₜ drives the latent state through the linear transformation represented by the input matrix B_{mode}. Each mode has its own separate B_{mode} matrix.

D_{mode}−inputₜ predicts neural activity yₜ through the linear transformation represented with the emissions matrix D_{mode}. Each mode has its own separate D_{mode}. In some examples, inputₜ occupies the dimensions of neural activity spanned by the columns of D_{mode}.

During the supervised calibration period, the subject can be placed in different discrete movement control modes and fit B_{mode}, D_{mode} in those modes. The disclosed system can decide which mode by determining which dynamics model yields the highest likelihood on the recent history neural activity. In some examples, the system controls the mode such that it does not switch sporadically. In other examples, some modes can be best fit in the supervised calibration and left constant, while other modes can be able to be continuously learned. In these examples, the user can choose which parameters can be fit only with supervised calibration and which parameters can be continuously learned.

In yet further embodiments, the neural dynamics model 204 comprises the following coefficients for the operation and update of the model 204:

$${z}_{t + 1} = {A}_{mode} {z}_{t} + {B}_{mode} {input}_{t}$$

$${x}_{t} = {C}_{mode} {z}_{t} + {D}_{mode} {input}_{t}$$

$${y}_{t} = {x}_{t} + {observation\_noise}_{t}$$

In this example, the neural dynamics model 204 switches between both discrete modes of inputs and discrete modes of recurrent dynamics.

A_{mode}−zₜ influences zₜ₊₁ through the linear transformation represented with the matrix A_{mode} (the recurrent dynamics matrix). Each mode has its own separate A_{mode} matrix.

C_{mode}−zₜ predicts neural activity yₜ through the linear transformation represented with the emissions matrix C_{mode}. Each mode has its own separate C_{mode} matrix. In some examples, zₜ occupies the dimensions of neural activity spanned by the columns of C.

In yet further embodiments, the neural dynamics model 204 comprises the following coefficients for the operation and update of the model 204:

$${latent\_state}_{t + 1} = A {latent\_state}_{t} + B {latent\_state}_{t}$$

$${neural\_state}_{t} = C {latent\_state}_{t}$$

$${neural\_input}_{t} = D {latent\_input}_{t}$$

$${neural\_denoised}_{t} = {neural\_state}_{t} + {neural\_input}_{t}$$

$${y}_{t} = {neural\_denoised}_{t} + {observation\_noise}_{t}$$

With the above coefficients in these embodiments, applying the neural dynamics model 204 can infer the state of neural activity that evolves with specific dynamics, and the input to the neural activity that modulates multiple neurons together. The inferred state and the input are two separate quantities that can in combination predict neural activity. In these embodiments, the neural dynamics model 204 can capture neural activity modulation in different dimensions, namely when it is driven by input, and when it follows recurrent dynamics respectively. The neural activity dimensions modulated by input are encoded in the matrix D in the model. The neural activity dimensions where activity follows recurrent dynamics are encoded in the matrix C in the model.

In some examples, the BMI decoding model 206 can take the inferred state and the input independently from each other and operate transformations on the inferred state and the input. Instead of receiving just a denoised neural activity signal “neural_denoisedₜ” as input (as in other examples), the BMI decoding model 206 receives two separate inputs: “neural_stateₜ” and “neural_inputₜ.” These two signals can carry different information, and the BMI decoding model 206 can solve and combine these two signals to produce a control signal for the BMI plant model 208.

FIG. 3 illustrates an example system 300 for processing brain activity information with models, in accordance with some embodiments. In the illustrated example, the system 300 includes a device 302 and a brain machine interface (BMI) 304. The BMI 304 is communicatively coupled to the device 302 via a communication channel. Examples of the BMI 304 generally include brain sensing devices or systems used to measure brain activity and/or record data corresponding to neural activities in the brain, such as non-invasive BMIs including electroencephalography (EEG), invasive BMIs including microelectrode array, and partially invasive BMIs including endovascular device. Examples of the device 302 generally include a computing device or system, such as personal computer, an on-premises server, a cloud-based server, or the like. Examples of the communication channel generally include wired and/or wireless links, and/or a network including one or more local area networks (LANs), wide area networks (WANs), wired networks, wireless networks, the Internet, or the like. In other embodiments, the BMI 304 is directly connected to and/or integrated within the device 302.

During operation of the BMI 304, it is configured to capture brain activities of a subject and record corresponding signals in brain activity data 306. Then, the BMI 304 sends the brain activity data 306 to the device 302 via the communication channel. In some embodiments, the device 302 is configured to control the operation of the BMI 304 to automate the brain activity measuring process. For example, the device 302 can send instructions to the BMI 304 to measure the subject's brain activity. The device 302 can further select parameters associated with the BMI 304, such as active time periods, sensitivity level, or the like.

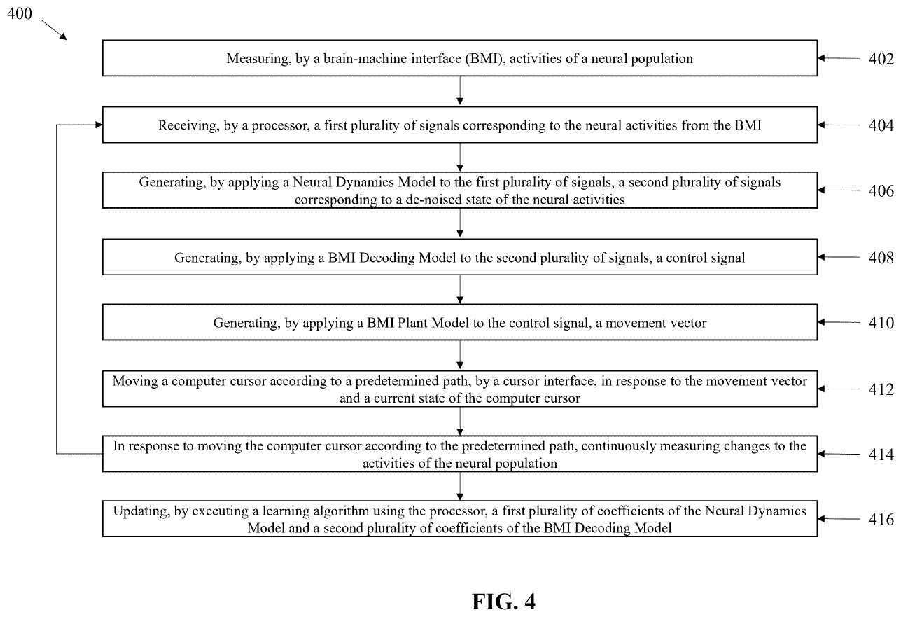

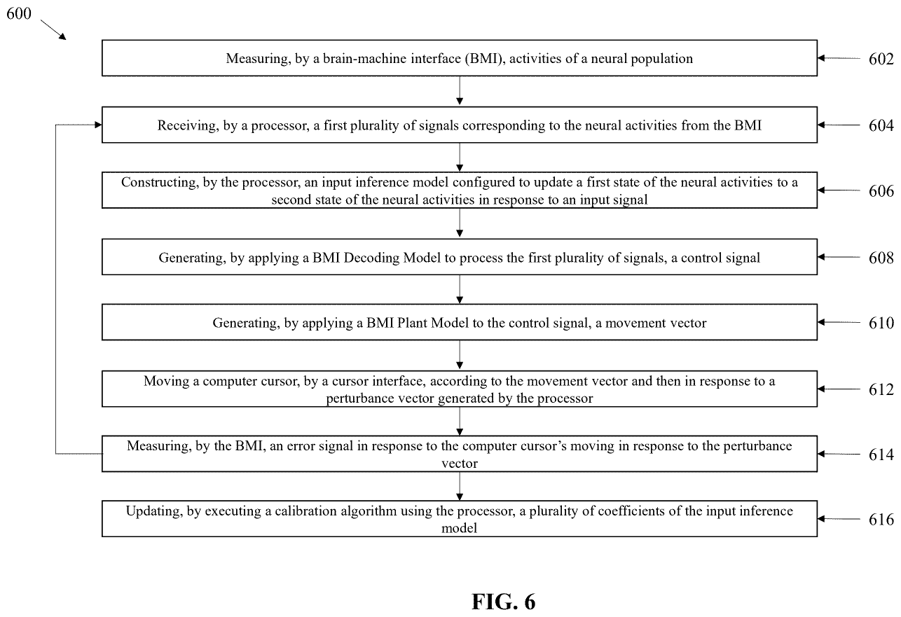

The device 302 includes a processor 310 (e.g., one or more hardware processors) coupled to a memory 320 (e.g., one or more non-transitory memories). The memory 320 stores instructions and/or data. When executed by the processor 310, the instructions and/or data stored by the memory 320 causes the processor 310 to perform operations associated with modeling and decoding neural activities (e.g., the operation of models and the transformation of signals described and illustrated in FIG. 2; also methods described and illustrated in below FIG. 4 and FIG. 6). Specifically, according to some embodiments, the memory 320 stores instructions and/or data corresponding to a neural dynamics model 324, a BMI decoding model 326, and a BMI plant model 328. In further embodiments, the memory 320 also stores instructions and/or data corresponding to a brain sensing program 202 and a cursor interface program 210 (described and illustrated in FIG. 2).

FIG. 4 illustrates an example method 400 for modeling and decoding neural dynamics, in accordance with some embodiments. In the illustrated example, the method 400 includes 402, 404, 406, 408, 410, 412, 414, and 416, which can be performed by one or more processors in conjunction with one or more memory devices (e.g., as described and illustrated in FIG. 3).

At 402 in FIG. 4, a BMI is configured to measure activities of a neural population in the brain of a subject. After measuring these neural activities, a processor is configured to receive a first plurality of signals corresponding to the neural activities from the BMI at 404.

Then, at 406, the processor is configured to apply a neural dynamics model and generate a second plurality of signals corresponding to at least a denoised state of the neural activities. In some embodiments, these signals include a de-noised neural signal corresponding to a de-noised state of neural activities. The neural dynamics model extracts latent states that represent specific spatiotemporal structure in the neural activity of the subject. Based on at least these latent states, the neural dynamics model is configured to remove the effect of noises and construct a de-noised neural signal. The de-noised neural signal corresponds to a de-noised version of the neural activities being decoded.

In turn, at 408, the processor is configured to apply a BMI decoding model and transform at least one of the second plurality of signals to a control signal. Using the control signal as input, the processor is further configured to apply a BMI plant model and output a movement vector for controlling a computer cursor to be displayed on a display device of a computing device. Accordingly, the movement vector is generated at 410.

At 412, the processor, by operating a cursor interface program, is configured to cause a computer cursor to move on the display device of the computing device. The movement of the computer cursor is controlled along a predetermined path. The movement of the computer cursor is carried out in response to the movement vector generated at 410 and a current state of the computer cursor. In response to the cursor movement at 412, the BMI is configured to continuously measure changes to the activities of the neural population at 414, which generate changes in the first plurality of signals. Thus, the example method 400 iteratively performs 404 and other subsequent methods. In the illustrated example, the movement of the computer cursor is controlled along a predetermined path during a supervised calibration process. After the supervised calibration process is completed, the subject can cause the computer cursor to move on the display device of the computing device according to trained procedures.

During these iterative steps, the processor is configured to update a first plurality of coefficients of the neural dynamics model and a second plurality of coefficients of the BMI decoding model at 416. In some embodiments, updating the first plurality of coefficients of the neural dynamics model comprises executing an unsupervised learning algorithm and/or a supervised learning algorithm. On the other hand, updating the second plurality of coefficients of the BMI decoding model comprises executing a supervised learning algorithm. After the supervised calibration process is completed, the processor is configured to update a first plurality of coefficients of the neural dynamics model while the BMI is configured to continuously measure changes to the activities of the neural population (e.g., as described at 414).

In a supervised calibration and/or learning period, the parameters of the neural dynamics model and the BMI decoding model are adapted jointly as the subject uses the BMI to control the cursor along the predetermined path with an initial set of parameters. As time elapses and the subject makes errors, an optimization procedure is configured to optimize the parameters to: (1) improve the neural dynamics model's accuracy in predicting future states of neural activities; and (2) reduce the error of the computer cursor.

In an unsupervised learning period, a subset or all of the parameters of the neural dynamics model can be updated as the subject operates the BMI. The unsupervised learning process is configured to improve the neural dynamics model's accuracy in predicting future states of neural activities.

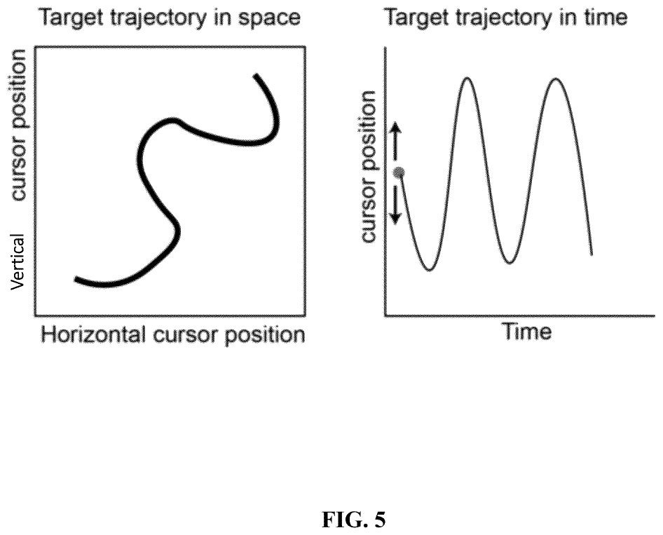

FIG. 5 is a non-limiting example illustrating a scenario applying the methods described and illustrated in FIG. 4, in accordance with some embodiments. In an example, the parameters of the neural dynamics model and the BMI decoding model are first adjusted in a supervised calibration period. This supervised calibration period involves specifying targeted movement trajectories which the subject is trained to follow by manipulating the computer cursor through the BMI. As depicted by FIG. 5, the targeted movement trajectories can be specified in space and/or in time. When specified in space, a target trajectory can be in a two-dimensional space with a horizontal cursor position and a vertical cursor position. When specified in time, a target trajectory can be in a two-dimensional space where one dimension is the cursor position that the subject is trained to follow, and the other dimension corresponds to time into the future. Accordingly, the subject is trained to manipulate the cursor to reach a target position at each point in time.

Neural activity can follow different dynamics when it controls movements with different properties (e.g., movements that oscillate with low or high frequencies, or movements that change directions fast versus slow). In some embodiments, a variety of target trajectories (e.g., that oscillate with low or high frequencies, or that change directions fast versus slow) are used to identify the parameters of the neural dynamics model across behavioral conditions. As the subject controls the cursor along these target trajectories, the neural dynamics model's parameters are adjusted to predict future neural activity based on past and present neural activity. In other embodiments, the BMI decoding model's parameters are adjusted to minimize a cursor error signal, which corresponds to the distance between the cursor's position and the target position. In the example scenario, the parameters of the neural dynamics model and the decoding model are being identified jointly and adjusted at the same time.

FIG. 6 illustrates an example method 600 for modeling and decoding neural dynamics, in accordance with some embodiments. In the illustrated example, the method 600 includes 602, 604, 606, 608, 610, 612, 614, and 616, which can be performed by one or more processors in conjunction with one or more memory devices (e.g., as described and illustrated in FIG. 3).

At step 602 in FIG. 6, a BMI is configured to measure activities of a neural population in the brain of a subject. After measuring these neural activities, a processor is configured to receive a first plurality of signals corresponding to the neural activities from the BMI at 604.

Then, at 606, the processor is configured to construct an input inference model configured to update a first state of the neural activities to a second state of the neural activities in response to an input signal. In some embodiments, at 606, the processor is also configured to apply a neural dynamics model and generate a second plurality of signals corresponding to at least a de-noised state of the neural activities. In some embodiments, these signals include a de-noised neural signal.

In turn, at 608, the processor is configured to apply a BMI decoding model and transform at least one of the first plurality of signals to a control signal, by processing with the input inference model and/or the neural dynamics model. In some embodiments, the BMI decoding model takes in both the output of the neural dynamics model and the output of the input inference model as inputs when generating the control signal. In some embodiments, the input inference model is a component of the neural dynamics model that, for example, infers an input variable (e.g., inputₜ). The inference of the input variable and the inference of a state variable (e.g., zₜ) are in conjunction with each other as operated by the neural dynamics model comprising the input inference model.

Using the control signal as input, the processor is further configured to apply a BMI plant model and output a movement vector for controlling a computer cursor to be displayed on a display device of a computing device. Accordingly, the movement vector is generated at 610.

At 612, the processor, by operating a cursor interface program, is configured to cause a computer cursor to move on the display device of the computing device. The movement of the computer cursor is controlled along a predetermined path. The movement of the computer cursor is carried out in response to the movement vector generated at 610 and a current state of the computer cursor. In addition, the movement of the computer cursor is carried out in response to a perturbance vector, which deviates from the predetermined path. Then, in response to the cursor movement at 612, the BMI is configured to measure changes to the activities of the neural population at 614, which generate an error signal in response to the computer cursor's moving in response to the perturbance vector. Accordingly, the example method 600 iteratively performs 604 and other subsequent methods.

During these iterative steps, the processor is configured to update the coefficients of the input inference model at 616 by executing a calibration algorithm. In some embodiments, updating these coefficients comprises executing an unsupervised learning algorithm and/or a supervised learning algorithm.

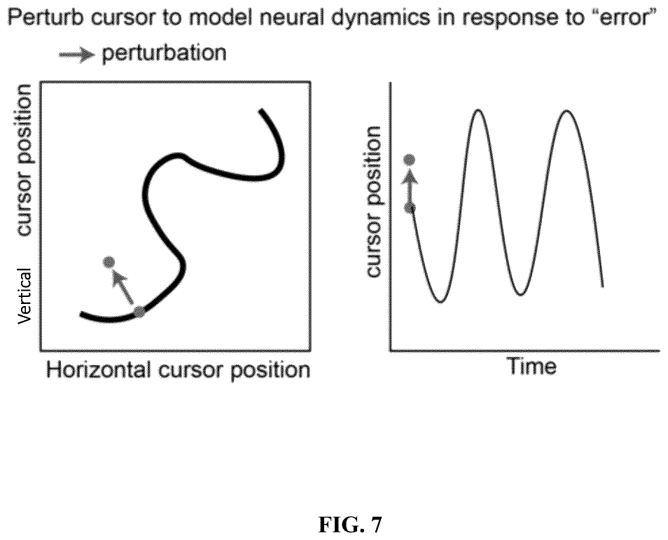

FIG. 7 is a non-limiting example illustrating a scenario applying the methods described and illustrated in FIG. 6, in accordance with some embodiments. In an example, a supervised calibration period involves introducing perturbations to the computer cursor which the subject is trained to correct. Processing these perturbations can improve identification of parameters of the neural dynamics model and the BMI decoding model. Perturbations to the cursor position, as depicted by FIG. 7, can cause the subject to use the feedback about the cursor's position. As a result, the disclosed systems and methods can infer how input signals and/or error signals drive neural activity. (e.g., to more accurately estimate parameters of models described above.) In the example scenario, the parameters of the neural dynamics model and the decoding model are being identified jointly and adjusted at the same time.

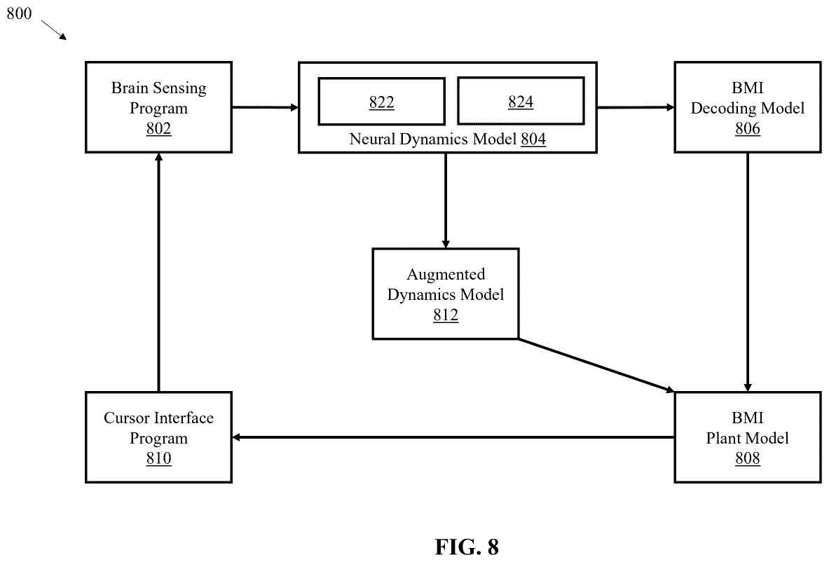

FIG. 8 illustrates an example decoding system 800 for decoding neural activities using a variety of models, in accordance with some embodiments. In the illustrated example, the decoding system 800 includes a brain sensing program 802, a neural dynamics model 804, a BMI decoding model 806, a BMI plant model 808, and a cursor interface program 810, which are also illustrated and described in FIG. 2. In addition, the decoding system 800 includes an augmented dynamics model 812. In some embodiments, the programs 802 and 810 as well as models 804, 806, 808, and 812 operate on one or more computing devices (e.g., the computing devices illustrated and described in FIG. 1).

The neurons in the brain of a subject send signals to the spinal cord in order to generate movement. Neurons in the spinal cord are connected in precise ways and generate rich dynamics, in addition to the dynamics present in the brain's neurons. The spinal cord's dynamics can be critical for generating movement. In analogy to the spinal cord's dynamics, the augmented dynamics model 812, when executed by a processor, provides additional decoding mechanism using rich dynamics that neurons in the brain can activate to help it control movement. For example, a subject using a BMI can move the computer cursor in a circular trajectory at a certain frequency. If the neurons in the subject's brain can't generate activity patterns that evolve like a circle at the desired frequency, then the computer cursor will likely move in a noisy manner that only approximates the circle with the predetermined frequency. However, by applying the augmented dynamics model 812 with the processor, improved controlling and decoding of dynamics can result in movements of the computer cursor in a circle at the predetermined frequency. In this example, neural activity is transformed by those augmented dynamics modeled by the augmented dynamics model 812.

In the illustrated example, the augmented dynamics model 812 takes the output of the neural dynamics model 804 as its input. The BMI plant model 808 takes both the output of the augmented dynamics model 812 and the output of the BMI decoding model 806 as its inputs. In other embodiments, the augmented dynamics model 812 also takes its input from the BMI decoding model 806. In an example, a BMI provides feedback to the subject about the state of the augmented dynamics, in addition to the state of the prosthetic that the subject attempts to control by using the BMI. This feedback can be provided through any modality (including visual, auditory, or haptic vibration, or direct stimulation of the brain). Accordingly, the augmented dynamics model 812 comprises the following coefficients for the operation and the optimization of the model 812:

$$\begin{matrix}a u {g}_{t + 1} = G a u {g}_{t} \\ {cursor}_{t + 1} = F {cursor}_{t} + {F}_{a u g} a u {g}_{t} + K {x}_{t}\end{matrix}$$

augₜ is the state of the augmented dynamics.

When combining the output of the BMI decoding model 806, the BMI decoding model 806 comprises additional coefficients:

$$a u {g}_{t + 1} = {K}_{a u g} {x}_{t} + G a u {g}_{t}$$

After the parameters of the neural dynamics model 804 and the BMI decoding model 806 are optimized, those parameters are used to solve for parameters of the augmented dynamics F_{aug} and K_{aug}. Solving these parameters reduces the theoretical magnitude of inputₜ that the neural dynamics model 804 would need to produce some target movements.

In some embodiments, in another supervised calibration period, with F_{aug} and K_{aug} initialized to this optimal solution, parameter of models 804, 806, and 812 can be adapted again in a closed loop to improve accuracy in predicting future neural activity and reduce the computer cursor's error. In other embodiments, before the supervised calibration period, parameters of the augmented dynamics model 812 can be adapted in response to specific movements of the computer cursor.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of nonvolatile memory, including by way of example semiconductor memory devices, (e.g., EPROM, EEPROM, and flash memory devices); magnetic disks, (e.g., internal hard disks or removable disks); magneto optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

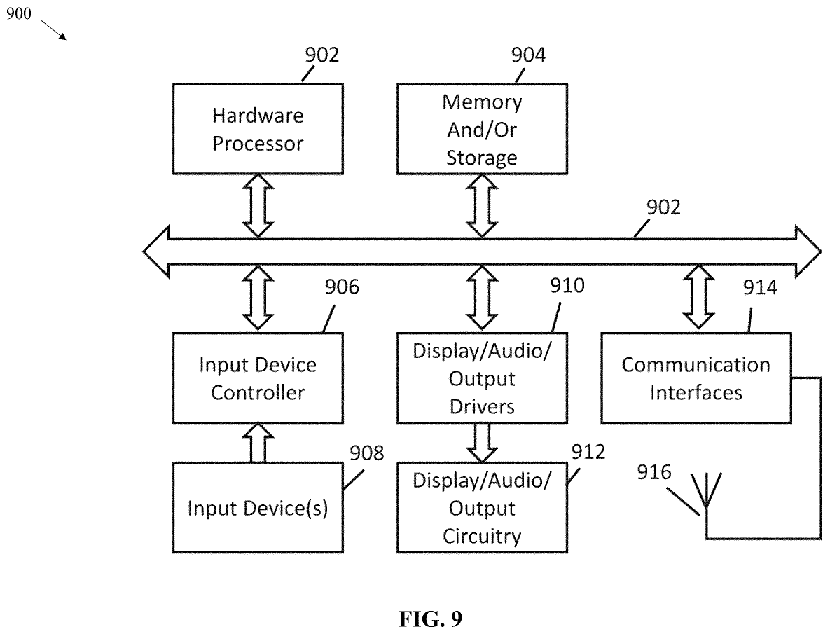

For example, FIG. 9 illustrates a computer hardware system 900 that can be used in accordance with some embodiments. The example computer hardware system 900 includes a hardware processor 902, memory and/or storage 904, an input device controller 906, an input device 908, display/audio/output drivers 910, display/audio/output circuitry 912, communication interface(s) 914, an antenna 916, and a bus 918. The hardware processor 902 can include any suitable hardware processor, such as a microprocessor, a micro-controller, a digital signal processor, dedicated logic, and/or any other suitable circuitry for controlling the functioning of a general-purpose computer or a special purpose computer in some embodiments. In some embodiments, multiple hardware processors 902 can be included to facilitate parallel processing as described herein.

The memory and/or storage 904 can be any suitable memory and/or storage for storing data, lookup tables, programs, etc. in some embodiments. For example, memory and/or storage 904 can include random access memory, read-only memory, flash memory, hard disk storage, optical media, etc. The input device controller 906 can be any suitable circuitry for controlling and receiving input from one or more input devices 908 in some embodiments. For example, the input device controller 906 can be circuitry for receiving input from a touch screen, from one or more buttons, from a voice recognition circuit, from a microphone, from a camera, from an optical sensor, from an accelerometer, from a temperature sensor, from a force sensor, from a near field sensor, a brain interface sensor, a muscle sensor, a nerve sensor, etc. The display/audio/output drivers 910 can be any suitable circuitry for controlling and driving output to one or more display/audio/output circuitries 912 in some embodiments. For example, the display/audio drivers 910 can be circuitry for driving an LCD display, a speaker, an LED, an industrial robot, a robotic arm, a robotic leg, a robotic hand, a robotic exoskeleton, a wheelchair, a stimulator, etc. The communication interface(s) 914 can be any suitable circuitry for interfacing with one or more communication networks, in some embodiments. For example, interface(s) 914 can include network interface card circuitry, wireless communication circuitry, etc. The antenna 916 can be any suitable one or more antennas for wirelessly communicating with a communication network in some embodiments. In some embodiments, the antenna 916 can be omitted when not needed. The bus 918 can be any suitable mechanism for communicating between two or more components 902, 904, 906, 910, and 914 in some embodiments. Any other suitable components can be included in the computer hardware system 900, and any un-needed components of the computer hardware system 900 can be omitted, in accordance with some embodiments.

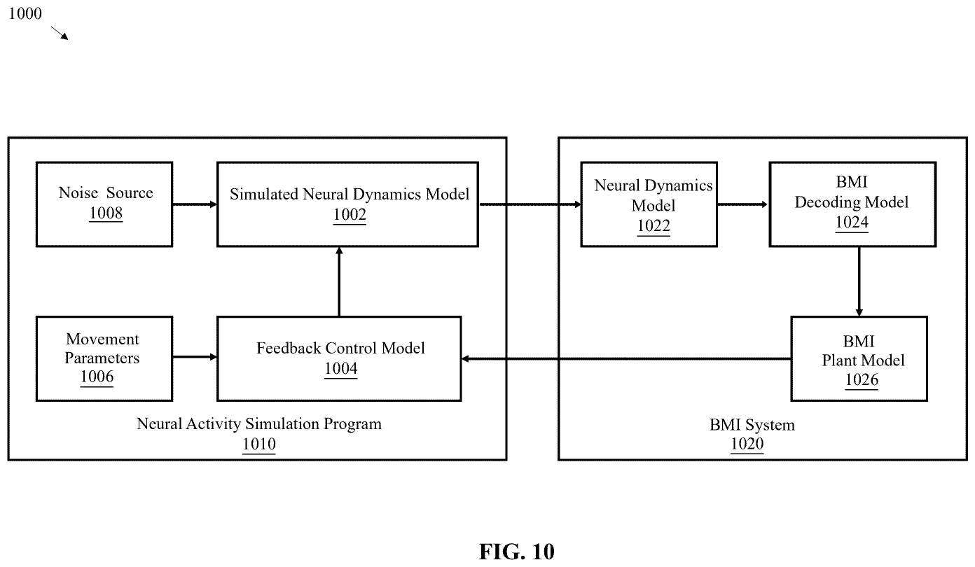

FIG. 10 illustrates an example simulation system 1000, in accordance with some embodiments. In the illustrated example, the simulation system 1000 comprises a neural activity simulation program 1010 and a BMI system 1020. The neural activity simulation program 1010 and the BMI system 1020 can operate on one or more processors in conjunction with one or more memory devices (e.g., as described and illustrated in FIG. 3). In some embodiments, the neural activity simulation program 1010 is configured to work in combination with the BMI system 1020. As a result, the simulation system 1000 can verify the operations of a BMI system (for example, test runs before mass production) without invasive surgeries for brain sensors to be implanted. Using the neural activity simulation program 1010 in combination with the BMI system 1020 helps designing improved BMI algorithms, and testing BMI systems before their deployment to human and/or animal subjects with expensive neural sensor implants.

In some embodiments, an example method comprises simulating a neural activity signal by the neural activity simulation program 1010 and controlling the BMI system 1020 (e.g., in a closed-loop process). In generating the simulated neural activity signal, an optimal feedback controller generates an input signal with a feedback control model 1004. In addition, the neural activity simulation program 1010 can generate a state signal by a simulated neural dynamics model 1002, which models more than a cursor's position and velocity. By combining the input signal and the state signal, the neural activity simulation program 1010 can simulate neural activities more accurately and control a BMI (e.g., the BMI system 1020) to perform specific predetermined movements. For instance, the BMI system 1020 can receive the simulated neural activity signal with a neural dynamics model 1022 in the BMI system 1020. In some examples, the simulated neural dynamics model 1002 also takes inputs from a noise source 1008. In other examples, the feedback control model 1004 also takes inputs from movement parameters 1006.

In other embodiments, another example method of simulating neural activity simulate how a subject's feedback control strategy interacts with a BMI in a closed-loop process, in which the subject uses physical movement to simulate controlling a BMI. For example, the example method involves using, in addition to the example simulation system 1000, a device configured to measure the subject's physical movement (e.g., the subject applies force on an immobile joystick). The physical movement being measured can be transformed by the disclosed system and method to a command signal for the cursor (e.g., a command for the velocity of a two-dimensional cursor). Then, by operating with a processor the neural activity simulation program 1010, which incorporates a model of neural dynamics, the neural activity simulation program 1010 can infer a simulated neural activity signal that would be generated to issue the command signal. The neural activity simulation program 1010 then sends the simulated neural activity signal to a BMI decoding model 1024 and a BMI plant model 1026 of the BMI system 1020. In turn, the cursor is updated, and its updated status is provided to the subject as feedback. The subjects can then update their movement based on the feedback to control the simulated BMI. The disclosed system and method can simulate how neural activity is generated to control a BMI based the subjects' own feedback control strategy. In other embodiments, the device configured to measure the subject's physical movement can function as the feedback control model 1004 in the example simulation system 1000.

A brain can generate a vast variety of movements. The brain would not have such capacity if it used separate populations of neurons to control each movement. Thus, the brain's capacity to produce different movements relies on re-using the dynamics of a specific neural population's activity. Neural activity is transmitted through recurrent connectivity, giving rise to neural population dynamics that can be re-used to control different movements. In some embodiments, systems and methods disclosed by the present application can identify that the brain re-uses dynamics to actually control movements.

The motor cortex of the brain, a region that controls movement through direct projections to the spinal cord and other motor control centers, is studied to show that neural population dynamics have similarity across different movements. Specifically, the spatial pattern of population activity at a given time point (e.g., the instantaneous firing rate of each neuron in the population) influences what spatial pattern occurs next, and this influence is similar across different movements. When similar activity patterns occur in different movements, the next pattern in time is similar across the different movements (a phenomena termed “untangled” neural activity). In some embodiments, models of neural population dynamics h that are invariant across movements³ can predict the transition from the current population activity pattern xₜ to the subsequent pattern xₜ₊₁:

$${x}_{t + 1} = h ( {x}_{t} ) + {input}_{t} + {noise}_{t}$$

In this example, the external input inputₜ and noise noiseₜ are typically unmeasured sources of neural activity variation. Such models have explained features of neural activity that were unexpected from measured behavior such as oscillations of particular components of neural activity. Dynamics models have predicted neural activity during different movements on single trials, for single neurons' spiking, for local field potential features, and over many days. These models have even helped predict ongoing behavior from neural activity.

In some embodiments, a test using the disclosed system can identify the causal transformation from neural activity to command, where the “command” is the instantaneous influence of the nervous system on movement. This is a long-standing and unsolved challenge in motor control, which is increasingly appreciated how complex the transformation is, with multiple pathways from motor cortex activity to muscle activation. To address the challenge, a brain-machine interface (BMI) is leveraged, in which the transformation from neural activity to command for movement was known exactly and determined by the experimenter, and in which the neural activity and commands were fully observed. Rhesus monkeys are trained to use the activity of 20-151 units in motor cortex (dorsal premotor and primary motor cortex) to move a two-dimensional computer cursor on a screen through a BMI. Analogous to motor control in which the nervous system transforms neural activity into muscle activation to apply force on the skeleton, the BMI transformed neural activity into a force-like command to update the cursor's velocity. Thus, an individual movement is produced by a series of commands, where each command acts on the cursor at an instant in time. Importantly, the BMI enabled identifying when the same exact command was issued across different movements, and to study how neural activity transitioned into and out of the pattern that issued the command.

If the instantaneous neural activity pattern both issues the command and influences the next pattern, it can be observed that the same exact command is issued with different neural activity patterns in different movements. Indeed, the neural pattern that issues a command is not invariant across movements. These different patterns transition according to low-dimensional, invariant dynamics to patterns that issue the next command, even when the next command differs across movements. Thus, the results demonstrate that the brain uses invariant dynamics to issue commands across movements.

Prior to the disclosed systems and methods, it had been unclear what invariant dynamics can provide for controlling movement. It had been unclear how this view would extend to movement that is controlled based on ongoing feedback. Indeed, motor cortex is interconnected to larger motor control circuits including cortical and cortico-basal ganglia-thalamic circuits, and ongoing input to motor cortex is relevant to its pattern generation during arm movement⁴³. Thus, a hierarchical model of optimal feedback control (OFC) can be introduced in which the brain (e.g., larger motor control circuitry) uses feedback to control the motor cortex population which controls movement. The model reveals that the invariant dynamics observed in experiments can help transform feedback into neural patterns for motor control, as they reduce the input that a neural population needs to issue commands based on feedback.

In some embodiments, results demonstrate that invariant neural population dynamics are both used and useful for issuing commands across different movements. These findings from BMI experiments show how invariant neural population dynamics can control physical movement. These results show how variable neural activity patterns for a fixed behavioral output can arise from invariant dynamics and provide a computational accuracy for controlling behavior.

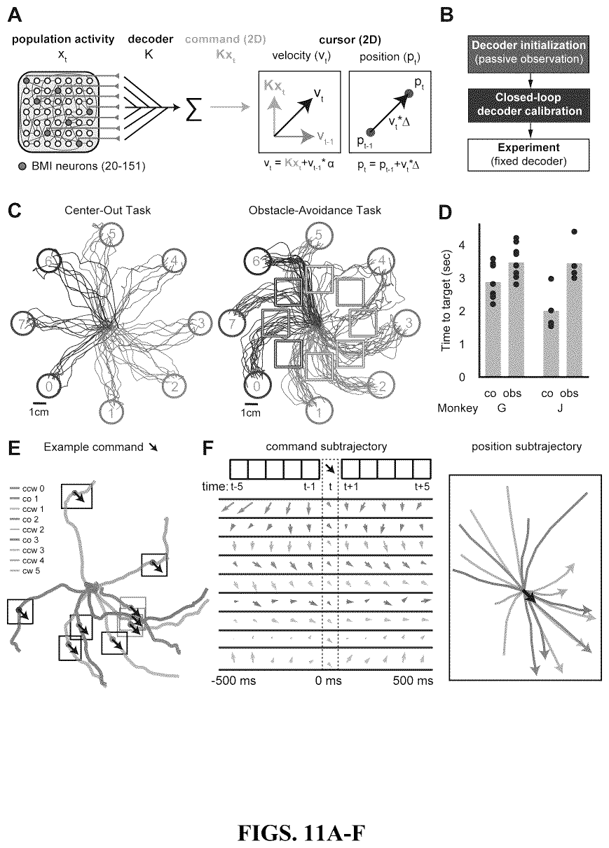

For studying neural population control of movement, a BMI is used to study the dynamics of neural population activity as it causally issued commands for movement of a two-dimensional computer cursor. The BMI transformed high-dimensional neural population activity into two-dimensional commands that update the computer cursor's velocity (FIG. 11A). Neural activity was recorded using chronically implanted microwire electrode arrays spanning bilateral dorsal premotor cortex and primary motor cortex. Each unit's spiking rate at time t (computed as the number of spikes in a temporal bin) was stacked into a vector of population activity xₜ, and the BMI used a “decoder” given by matrix K to linearly transform population activity into a two-dimensional command:

$${command}_{t} = K {x}_{t}$$

The command linearly updated the two-dimensional velocity vector of the computer cursor (FIG. 11A right and Methods):

$${velocity}_{t} = {command}_{t} + α * {velocity}_{t - 1} + offset$$

The BMI was not identical across the two subjects, as neural activity was modeled with different statistical distributions (Gaussian for Monkey G and a Point Processs for Monkey J). Nonetheless, both the command update equation (Equation 2) and the velocity update equation (Equation 3) hold for both subjects (see STAR methods—“Neuroprosthetic decoding”).

One experimental design for defining the decoder allowed studying how the neural population issued commands specifically for BMI movement (FIG. 11B). On each session, a decoder was initialized from a baseline recording block in which subjects passively watched a cursor move through the tasks. Then in a closed-loop decoder calibration block, the decoder was continuously refined based on subjects' control of the BMI in closed-loop. Following calibration, the decoder was fixed, and the experiment began. All data subsequently analyzed was from fixed-decoder blocks. Under this experimental design, subjects controlled the BMI without using trained overt movement. Critically, this BMI did not demand movement-associated neural population dynamics because the BMI was not designed to decode neural activity during trained overt movement, as was done previously.

To study neural population control of diverse movements, monkeys can be trained to perform two different tasks (FIGS. 11C, 11D). Monkeys performed a center-out task in which the goal was to move the cursor from the center of the workspace to one of eight radial targets, and they performed an obstacle-avoidance task with the same goal plus the constraint of avoiding an obstacle blocking the straight path to the target. The behavior paradigm elicited up to 24 conditions of movement (with an average of 16-17 conditions per session), where each condition is defined as the task performed (“co”=center-out task, “cw”/“ccw”=clockwise/counterclockwise movement around the obstacle in the obstacle-avoidance task) and the target achieved (numbered 0 through 7).

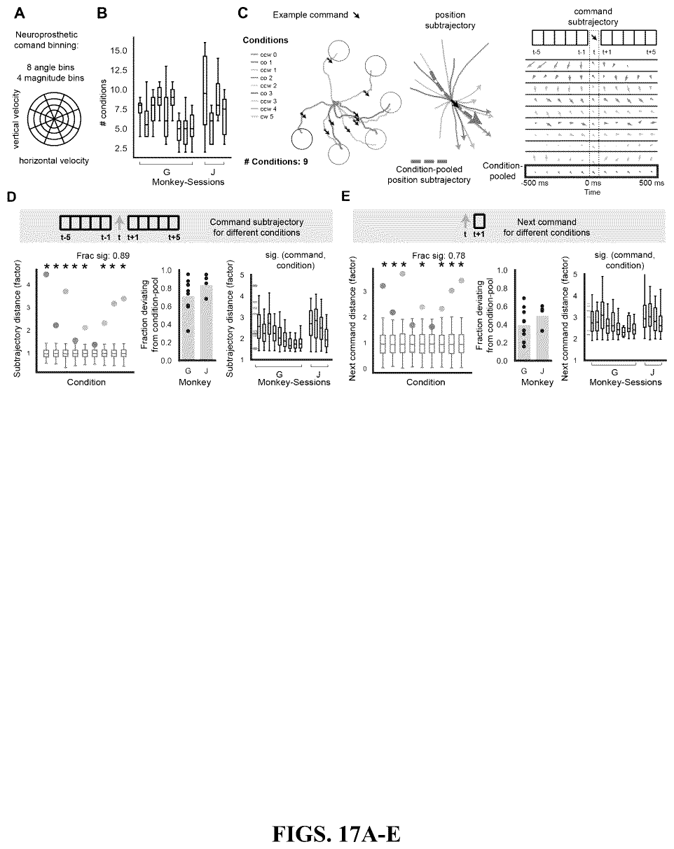

The BMI enabled identifying when the same exact command was issued in different conditions (FIGS. 11E, 11F, FIGS. 17A-17E). This allowed studying neural population activity when it exerted the same instantaneous influence on behavior across different conditions. For analysis, particular two-dimensional, continuous-valued commands are considered as the same if they fell within the same discrete bin. Commands are categorized into 32 bins (8 angular×4 magnitude) based on percentiles of the continuous-valued distribution (FIG. 17A: see STAR methods—“Command discretization for analysis”). On each session, a command (of the 32 discretized bins) was analyzed if it was used in a condition 15 or more times (FIG. 17B). Each individual command was used with regularity during multiple conditions (on average ˜7 conditions, FIG. 17B), within distinct local “subtrajectories” of previous and subsequent commands and cursor positions (FIG. 11F, FIGS. 17A-17E, STAR methods—“Cursor and command trajectory visualization”).

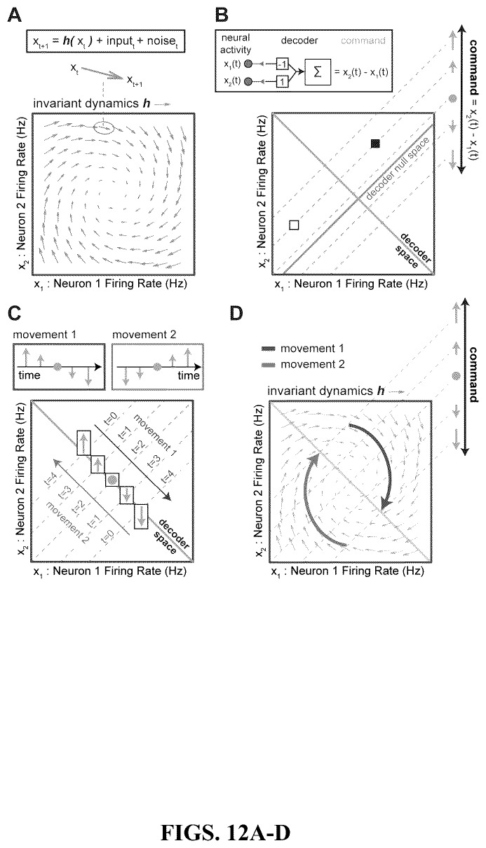

For using the BMI to test whether the brain uses invariant dynamics to control different movements, the BMI enabled testing whether the brain uses invariant dynamics to issue commands for different movements. If it does, the current neural activity pattern can influence the subsequent pattern in an invariant manner (Equation 1, FIG. 12A), such that the current pattern both issues the current command (Equation 2, FIG. 12B) and influences the subsequent command. An activity pattern xₜ can be visualized as a point in high-dimensional neural activity space, where each neuron's activity is one dimension, and visualize the transition between two patterns xₜ and xₜ₊₁ as an arrow (FIG. 12A). Then, dynamics that are invariant across different movements can be visualized as a flow field in neural activity space, where the arrows of the flow field illustrate predicted transitions in neural activity. This flow field is invariant because the predicted transition for a given neural activity pattern does not change, regardless of the current command or condition of movement. If the brain uses invariant dynamics to issue commands, neural activity trajectories can follow along the flow of invariant dynamics.

The BMI allowed studying what neural activity patterns were used to issue a command. The brain can use many different neural activity patterns to issue a particular command through the BMI, as is believed to be true in the natural motor system. This is because there are more neurons than command dimensions, as illustrated with two neurons and a one-dimensional command (FIG. 12B). The BMI decoder defined dimensions of neural activity that determine the command. These dimensions can be also referred to as the “decoder space,” and the orthogonal dimensions which have no consequence on the command through the decoder can be also referred to as the “decoder null space.” Concretely, neural activity's component in the decoder space determines the command issued.

The BMI allowed observing the precise temporal order of commands used to produce an individual movement, as illustrated for two cartoon movements (FIG. 12C). Because of the decoder-null space, many different neural trajectories would produce a given movement. It is tested whether neural activity trajectories followed the flow of invariant dynamics to issue commands across different movements (FIG. 12D).

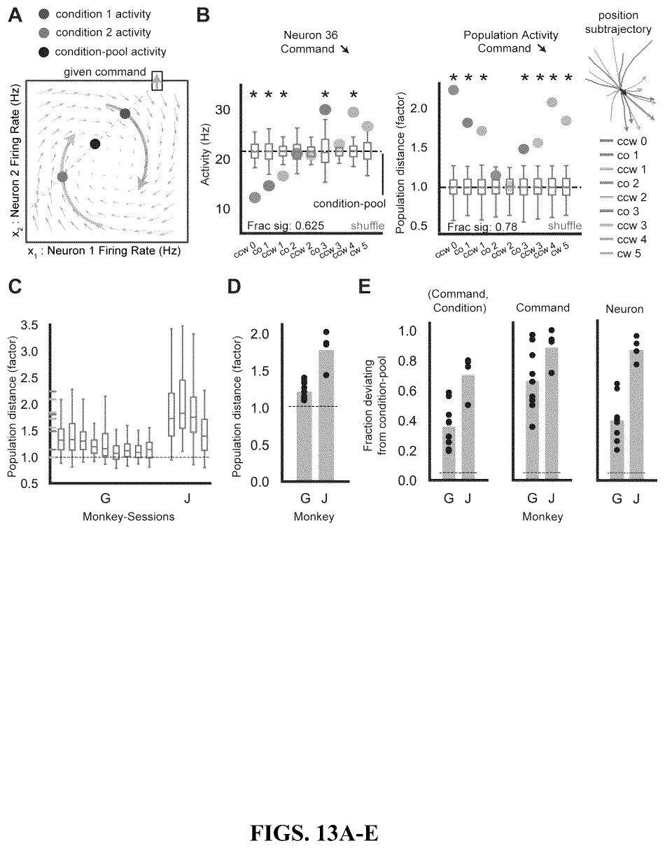

During BMI movement, the same command was issued in different conditions and was followed by different subsequent commands (FIG. 11F, FIGS. 17D, 17E). If the current neural activity pattern not only issues a command but also influences the subsequent pattern, then the same command can be issued by different neural activity patterns in different conditions (FIG. 13A). Thus, the distance between the average neural activity is calculated for a given command in a given condition (“condition-specific average activity”) and the average neural activity for a given command pooled over all conditions (“conditioned-pooled average activity”). Then it is tested whether this “condition-specific distance” is larger than expected simply due to the variability of noisy neural activity. To emulate the scenario in which neural activity for a given command is drawn from the same, noisy distribution across conditions, shuffled datasets are constructed where all observations of neural activity are identified issuing a given command and shuffled their condition-labels, for all commands (see STAR methods—“Behavior-preserving shuffle of activity”). In this scenario, the condition-specific distance is expected to be greater than zero simply because condition-specific average activity is estimated from limited samples and thus is subject to variability. Thus, it is tested whether the experimentally-observed condition-specific distances exceeded the distribution of distances calculated on the shuffled datasets (see STAR methods—“Analysis of activity issuing a given command”).

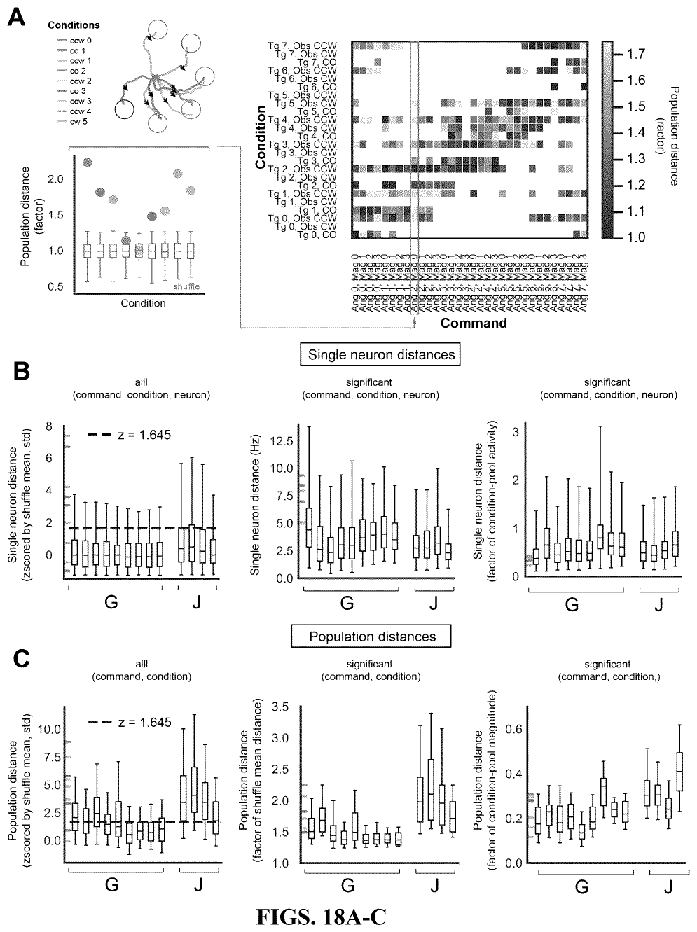

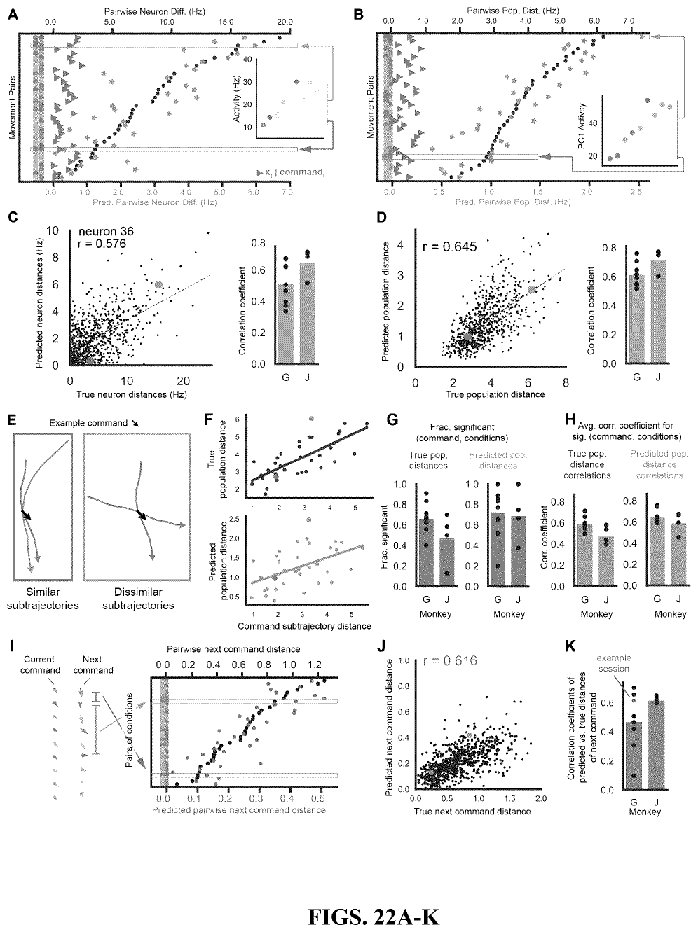

Overall, neural activity issuing a given command significantly deviated across conditions relative to the shuffle distribution (FIGS. 13B-13E). This is illustrated for an example command for an example neuron (FIG. 13B left) and the entire population (FIG. 13B right). The entire distribution of condition-specific population activity distances is shown for each session, normalized to the mean of the shuffle distribution (FIG. 13C). Condition-specific distances for individual sessions ranged from 10% to 200% larger than the average shuffle distance (FIG. 13D). Distinct activity for a given command was detectable not only at the level of the neural population (stats in FIG. 13D legend), but also for individual neurons (stats in FIG. 13B legend). Sec FIGS. 18A-18C for additional distance distributions and normalizations, including distributions of the specific (command, condition) tuples that significantly deviate from shuffle. Condition-specific distances were significantly larger than shuffle distances for a large fraction of individual (command, condition) tuples (˜30% for Monkey G, ˜70% for Monkey J, FIG. 13E left), individual commands (˜65% for Monkey G, ˜90% for Monkey J, FIG. 13E middle) when aggregating over conditions, and individual neurons (˜40% for Monkey G, ˜80% for Monkey J, FIG. 13E right) when aggregating over all (command, condition) tuples. Further, these deviations reflected structure in the behavior, as neural activity issuing a given command was more distinct for conditions with more distinct past and future commands (FIGS. 22E-22H). This evidence demonstrates that different neural activity patterns are used to issue the same command in different conditions.

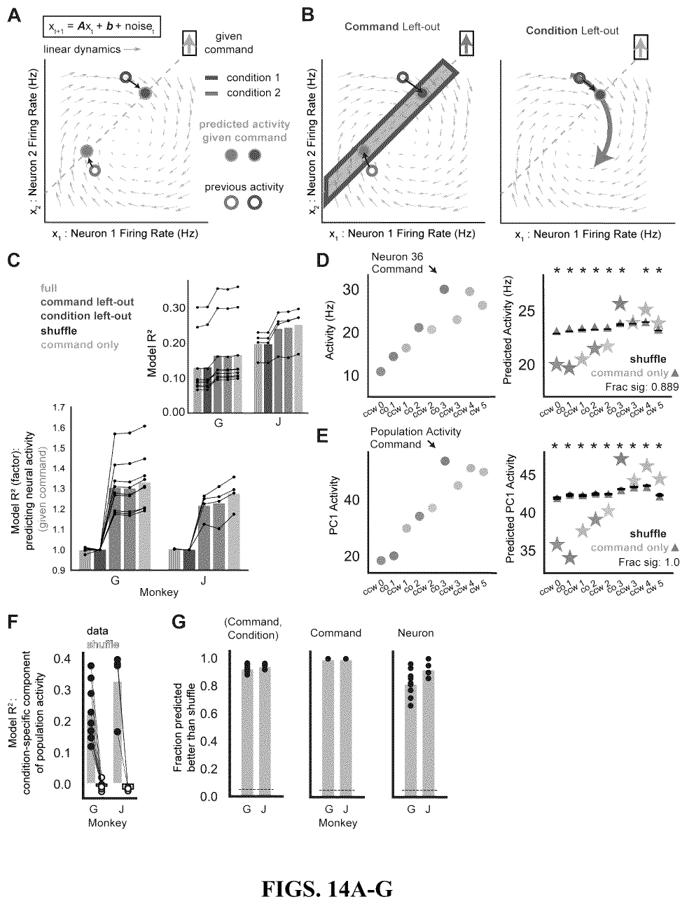

Invariant dynamics can predict the different neural activity patterns used to issue the same command. Given that the specific neural activity pattern that issued a command was not invariant across conditions, a model of invariant dynamics is constructed next. Single-trial neural activity xₜ from all conditions is used to estimate neural population dynamics with a linear model (FIG. 14A):

$${x}_{t + 1} = A {x}_{t} + b$$

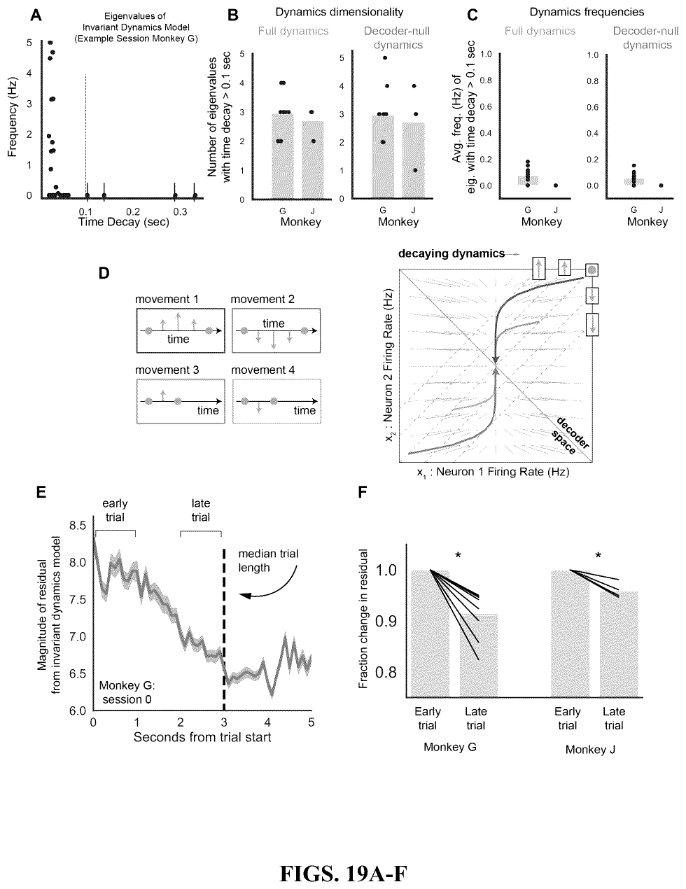

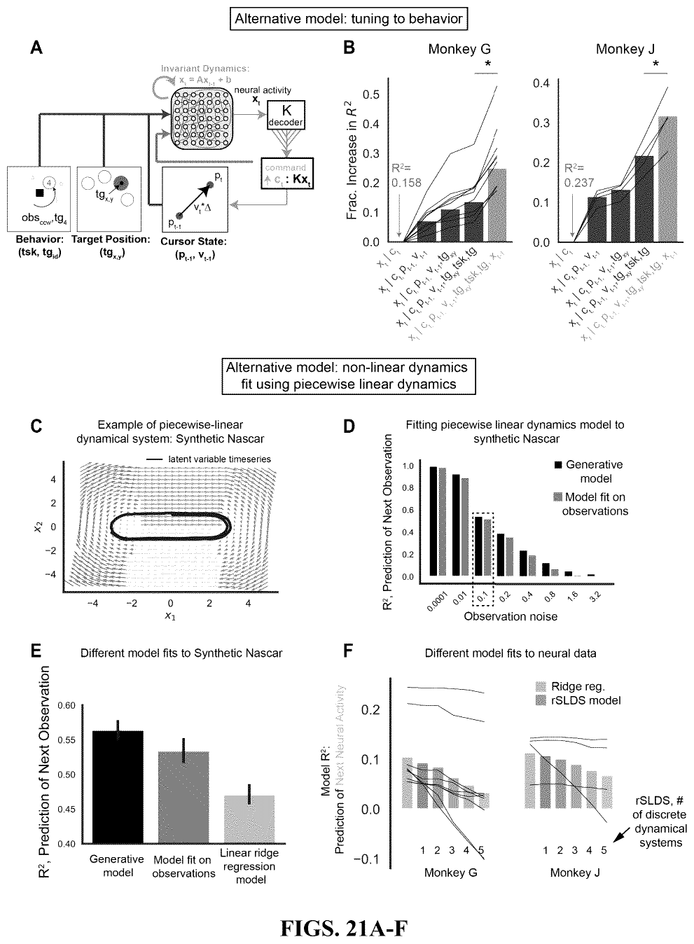

The dynamics A were low-dimensional (2-4 dimensions) and decaying to a fixed point (FIGS. 19A-19C), contrasting with rotational dynamics observed during natural motor control ^{12,13,16,22,51}. See FIG. 19D for an illustration of how decaying invariant dynamics can control different movements. Notably, a state-of −art non-linear dynamics model (a recurrent switching linear dynamical system⁵²) did not out-perform these simple linear dynamics (FIGS. 21C-21F).

To address the question whether invariant dynamics predict the different neural activity patterns issuing the same command, the dynamics model (Equation 4) is combined with knowledge of the BMI decoder (Equation 2) to predict the population's activity pattern given the command it issued and its previous activity (FIG. 14A, see STAR methods—“Invariant dynamics model predictions”). This approach constrained the invariant dynamics model's prediction of activity to necessarily issue the given command. Thus, the model's prediction accuracy was interpretable for addressing the question, as errors weren't simply due to predicting activity patterns that issued the wrong command. For comparison to the invariant dynamics model, the prediction of neural activity is also computed when only given the command it issued (in the absence of a dynamics model).

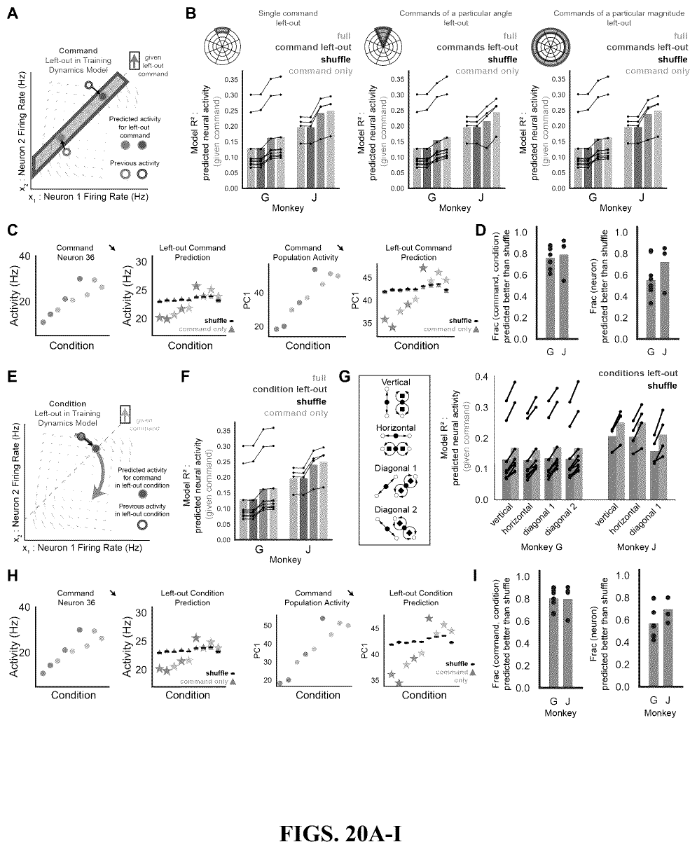

Further, it is tested whether the invariant dynamics model generalized to new commands and conditions. To test generalization, dynamics models were fit on neural activity specifically excluding individual commands or conditions, and these models were used to predict the neural activity for the left-out commands or conditions (FIG. 14B, FIGS. 20A-201, see STAR methods—“Invariant dynamics models”).

It is tested whether the dynamics model's accuracy exceeded a dynamics model fit on shuffled datasets that preserved behavior and represented the re-use of the same noisy distribution of neural activity patterns to issue a command across movements. As described above, shuffled datasets are constructed to shuffle the condition-label of neural activity for a given command; this preserves the temporal order of commands while shuffling the neural activity issuing the commands (see STAR methods—“Behavior-preserving shuffle of activity”). The shuffle dynamics model captured the expected predictability in neural activity due to the behavioral structure of the performed movements.

On the level of single time points in individual trials, the dynamics model predicted the neural activity pattern issuing a given command based on the previous pattern (FIG. 14C), significantly exceeding the accuracy of shuffle dynamics. Further, the invariant dynamics model generalized across left-out commands and conditions, as it significantly exceeded shuffle dynamics and was close to the dynamics model trained on all commands and conditions (FIG. 14C). Notably, the model generalized even when much larger subsets of commands and conditions were left-out (FIGS. 20A-201). The result was not driven by neural activity simply representing behavioral variables in addition to the command, as dynamics models predicted neural activity better than models where neural activity encoded cursor kinematics, target location, and condition (FIGS. 21A-21B). This is consistent with previous work showing that cursor kinematics such as cursor position only weakly affect neural activity controlling a BMI in the absence of arm movements.

For each condition, the invariant dynamics model also predicted the different average activity patterns issuing a given command, as shown for an example command across conditions for the example neuron (FIG. 14D) and for the entire population (FIG. 14E). Invariant dynamics predicted 20-40% of the condition-specific component of average neural activity for a given command (e.g., the difference between condition-specific average activity and the prediction of that activity based on the command alone), across subjects and sessions (FIG. 14F, see STAR methods—“Invariant dynamics model predictions”, “Predicting condition-specific component”). The invariant dynamics model's accuracy for predicting condition-specific average neural activity significantly exceeded shuffle dynamics for almost all (command, condition) tuples (FIG. 14G left), commands (FIG. 14E middle) when aggregating over conditions, and neurons (FIG. 14E right) when aggregating over all (command, condition) tuples.

Finally, the invariant dynamics predictions of neural activity for a given command preserved structure between pairs of conditions (FIGS. 22A-22D) and explained the finding that the activity patterns for a given command are more distinct between pairs of conditions with more distinct past and future commands (FIGS. 22E-221). Altogether, these results show that invariant dynamics contribute to what activity pattern was used to issue a command, generalizing across commands and conditions.

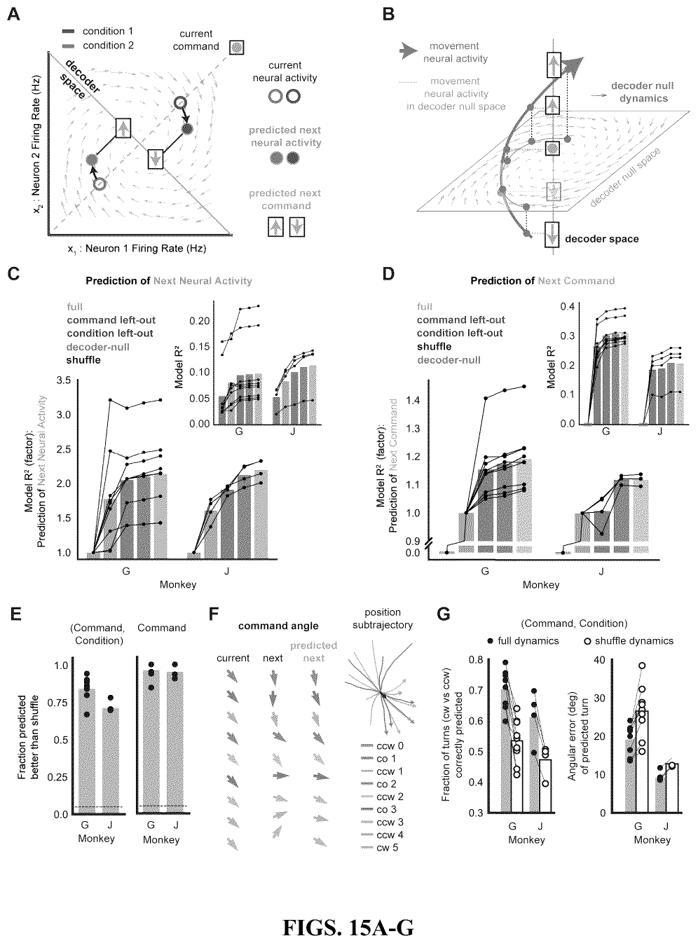

Invariant dynamics align with the decoder, propagating neural activity to issue the next command. For the question whether invariant dynamics were actually used to transition between commands, it can be determined that the consequence of invariant dynamics on the next command using knowledge of the BMI decoder. More specifically, the dynamics model is used to predict next neural activity from current neural activity, and then applied the decoder to predict the next command (FIG. 15A). This analysis was possible because the BMI decoder provides a fully-defined causal transformation from neural activity to command which has not been measurable during natural motor control.

Invariant dynamics can potentially not contribute to the next command. Invariant dynamics would not update the issued command (FIG. 15B) if invariant dynamics only transitioned neural activity in the decoder null space (e.g., the dimensions of neural activity that the decoder does not use to transform neural activity into a command, FIG. 12B). To assess this possibility, an invariant dynamics model is fitted on the component of neural activity in the decoder null space (“decoder-null dynamics”, see STAR methods—“Invariant dynamics models”, “Decoder-null dynamics model”). While this decoder-null dynamics model was restricted to the decoder-null space, it maintained very similar dimensionality and eigenvalues to the full dynamics model (FIGS. 19B-19C). Thus, this decoder-null dynamics model is compared to the full dynamics model.

On the level of single time points in individual trials, the decoder-null dynamics model's accuracy (FIG. 15C, pink bar) significantly exceeded shuffle dynamics (FIG. 15C, black bar), showing that neural activity transitions followed invariant dynamics in decoder-null dimensions. In contrast, the decoder-null dynamics model provided no prediction for the next command (FIG. 15D), as expected. This result shows that successful prediction of next neural activity does not imply prediction of next command.

Critically, the full dynamics model predicted both the next neural activity pattern (FIG. 15C, cyan bar) and the next command (FIG. 15D, orange bar), in contrast to decoder-null dynamics. Neural activity and command predictions significantly exceeded shuffle dynamics which captures expected predictability due to behavioral structure of movements. Importantly, the invariant dynamics model generalized across commands (FIGS. 15C-15D, magenta bar) and conditions (FIGS. 15C-15D, purple bar) that were left-out from model fitting. Together, these results demonstrate that invariant dynamics predict the current neural activity pattern's transition to the next activity pattern and command, on the level of single time points in individual trials,

When analyzed at the level of an individual command and condition, a particular command can be issued by distinct average neural activity patterns across different conditions. Thus, when analyzed at this level of individual commands and conditions, in studying whether the invariant dynamics model transitioned these distinct activity patterns towards distinct next commands appropriate for each condition, the dynamics model predicted the next command following a given current command, significantly exceeding shuffle dynamics for the majority of (command, condition) tuples (FIG. 15E left) and for the majority of commands (FIG. 15E right) when aggregating over conditions. These predictions preserved structure across pairs of conditions, such that invariant dynamics can indicate whether the next command would be similar or different across pairs of conditions (FIGS. 221-22K).

In studying whether invariant dynamics can predict interpretable properties of the next command, the direction of an example command, the next command, and the predicted next command are visualized all within different conditions (FIG. 15F). This is for analyzing whether invariant dynamics predicted the turn the next command would take in individual conditions (FIG. 15G). This turn angle was calculated between the next command in a given condition and the average next command pooling across conditions. The invariant dynamics model predicted both whether the turn would be clockwise or counter clockwise (FIG. 15G left) and the angle of turn (FIG. 5G right) for individual (command, condition) tuples better than shuffle dynamics. Altogether, these results show that invariant dynamics are used to transition between commands.

An OFC model reveals that invariant dynamics reduce the input that a neural population needs to issue commands based on feedback. The invariant dynamics model did not perfectly predict transitions between commands. Early in trials, neural activity exhibited large residuals from the dynamics model, consistent with inputs setting an initial state for movement (FIGS. 19E-19F), but throughout movement, there were also substantial residuals consistent with ongoing input driving neural activity in addition to invariant dynamics.

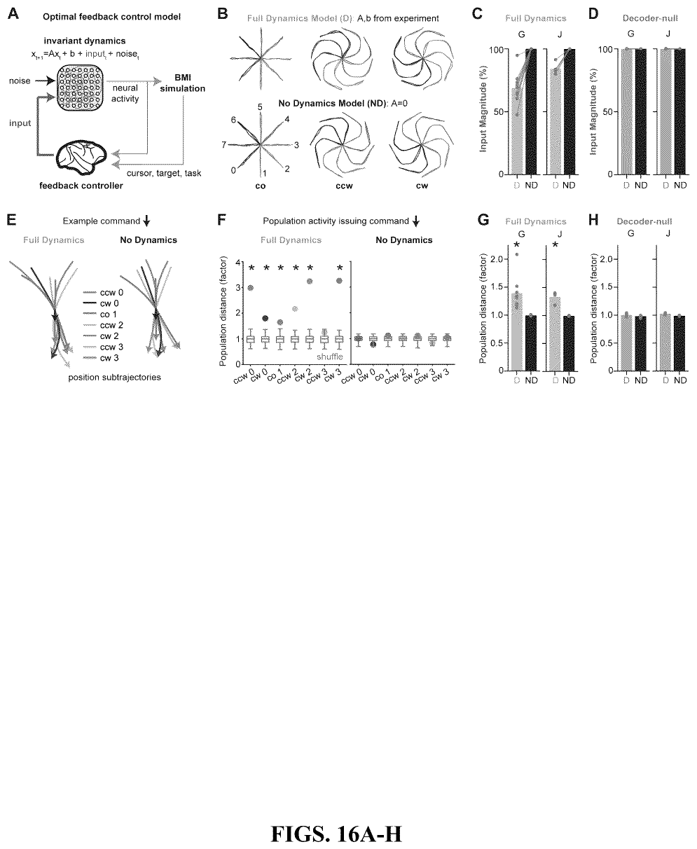

Given that motor cortex is interconnected to larger motor control circuits and that it needs ongoing input to generate activity for movement, a hierarchical model of optimal feedback control (OFC) is introduced in which the brain controls the motor cortex population which controls BMI movement (FIG. 16A). Concretely, in this model, the brain acts as an optimal linear feedback controller with knowledge of the neural population's invariant dynamics, the BMI decoder, and the movement's task and target. The brain transforms ongoing cursor state and population activity into the minimal input necessary to the neural population to achieve successful movement. The combination of this input, invariant dynamics, and noise results in the population activity that issues commands for movement (Equation 1). The model performing center-out and obstacle-avoidance movements is simulated with the BMI decoders that were used in experiments (see STAR methods—“Optimal feedback control model and simulation”).

To study the function of the neural population's invariant dynamics during feedback control, OFC models are simulated with and without invariant dynamics. If invariant dynamics help transform feedback into commands for movement, they can reduce the input that the neural population needs during feedback control. In the Full Dynamics Model, the brain computed the minimal input necessary to a neural population that followed the invariant dynamics observed experimentally. In the No Dynamics Model, the brain computed the minimal input necessary to a neural population that has no invariant dynamics, e.g., the dynamics matrix is set to zero. To facilitate comparison, the models are designed to receive the same magnitude of noise and to produce matched behavior, resulting in center-out and obstacle-avoidance cursor trajectories with equal success and target acquisition time (FIG. 16B).

These simulations revealed that, in some implementations, the neural population takes in significantly less input in the Full Dynamics Model than in the No Dynamics Model (FIG. 16C). This effect is not true for all types of invariant dynamics; it relied on the fact that the experimentally-observed invariant dynamics aligned with the BMI's decoder. The effect was erased in the Decoder-Null Dynamics Model (FIG. 6D), in which the OFC model's invariant dynamics were restricted to the decoder-null space (but still maintained similar dimensionality and eigenvalues, see FIGS. 19B-19C). These results show that invariant dynamics that specifically align with the decoder, as experimentally-observed, can help the brain perform feedback control, reducing the input the neural population needs to issue commands based on feedback.

Finally, the principle that using invariant dynamics to perform feedback control makes use of distinct neural activity patterns to issue a particular command. As in FIGS. 13A-13E, the simulated neural activity from the OFC models is compared against the behavior-preserving shuffle of neural activity that represents the re-use of the same activity distribution to issue a command across conditions. For an example command used during different conditions of the simulations (FIG. 16E), the distance between condition-specific average activity and condition-pooled average activity (the condition-specific distance) was significantly larger than shuffle in the Full Dynamics Model but not the No Dynamics Model (FIG. 16F). Indeed, Having analyzed activity over all (command, condition) tuples, the condition-specific distance in the Full Dynamics Model was significantly larger than shuffle, while there was no detected difference between the No Dynamics Model and shuffle (FIG. 16G). Further, this effect depended on alignment between invariant dynamics and the decoder, as there was no detected difference between the Decoder-Null Dynamics Model and shuffle (FIG. 16H). Thus, the OFC model used different neural activity patterns to issue the same command only when the invariant dynamics were useful for feedback control.

The way that neural activity is transmitted through the brain is constrained by its connectivity. Theoretical work shows that recurrent connectivity can give rise to dynamics of neural activity for motor control and endow the brain with the capacity to generate diverse physical movement. Important experimental work has found that neural population activity in the motor cortex follows similar and predictable dynamics across different movements. But the transformation from neural activity to command for movement has been challenging to measure; previous studies approximate the transformation with a model. Thus, it has been untested whether dynamics that are invariant across movements are used to actually control movement, or whether they are a consequence of movement. Here, that test is performed using a BMI to define the transformation from neural activity to command for movement of a neuroprosthetic cursor.

Different neural activity patterns are used to issue the same command in different movements. The neural activity patterns issuing the same command vary systemically depending on past neural activity, and critically, they transition according to invariant dynamics towards neural activity patterns that causally drive the subsequent command. The results' focus on the command provides a conceptual advance beyond previous work that characterized properties of dynamics during behavior, revealing that invariant dynamics are actually used to control movement.

A hierarchical model of optimal feedback control is introduced, in which the brain uses movement feedback to control a motor cortex population that controls movement. Optimal control theory reveals that invariant dynamics that are aligned to the decoder can help the brain perform feedback control of movement, reducing the input that a neural population needs to issue the appropriate commands based on feedback. The use of invariant dynamics for feedback control results in issuing the same command with different neural activity patterns across different movements, as observed in the experimental data.

These results provide strong evidence against one traditional view that the brain reuses the same neural population activity patterns to issue a particular command. This perspective is present in classic motor control studies that describe populations of motor cortex neurons as being dedicated to representing movement parameters that their activity updates. It is still debated what movement parameters are updated by motor cortex populations, as population activity encodes diverse movement parameters spanning from low-level muscle-related parameters such as muscle activation, muscle synergies, and force/torque to high-level movement parameters such as position, distance, velocity, speed, acceleration, and direction of movement. Recent work has discovered flexible neural control of even finer motor output at the level of motor units. Regardless of how motor cortex commands update physical movement, the findings using a BMI strongly suggest that the motor cortex does not use an invariant neural activity pattern to issue a specific motor command. The findings support the recent proposal that neural activity in motor cortex avoids “tangling”: having similar activity patterns undergo dissimilar transitions to control movement. Of course, whether the brain uses invariant dynamics to issue muscle commands remains to be demonstrated.

A neural population's invariant dynamics do not perfectly determine its next issued command, contrasting with the view that neural population dynamics evolve an initial state of activity into a complete activity trajectory which produces movement. Instead, a model can be used based on optimal control theory in which the neural population combines ongoing external input with invariant dynamics to control movement. As the brain sends input to the neural population, it performs feedback control on the state of the neural population's invariant dynamics in order to produce movement. In this view, the invariant dynamics may not need to define the precise neural activity transitions that perfectly produce movement; they only need to provide useful neural activity transitions that inputs can harness to control movement. The results show that invariant dynamics can reduce the input a neural population needs to issue commands based on feedback, adding to previous work identifying that invariant dynamics provide robustness to noise.

This perspective in which the brain performs feedback control on the state of a neural population's dynamics expands the number of behaviors for which invariant dynamics arc useful. This is because invariant dynamics can provide useful neural activity transitions for issuing commands across diverse movements without specifying the precise transitions needed to completely produce each movement. In the data, simple dynamics (decaying dynamics with different time constants) in a low-dimensional activity space (˜4 dimensions) were used to control many conditions of movement (˜20 conditions). While the results did not rely on high-dimensional neural dynamics, they still rely on more dimensions of neural activity than command dimensions. In particular, the results refute a simplistic interpretation of the minimal intervention principle in which neural populations can only control the few dimensions of neural activity which matter directly for issuing commands, which are given by the decoder space in these experiments. Instead, invariant dynamics provide constraints in the dimensions of neural activity which do not directly matter for issuing current commands, given by the decoder null space (FIG. 12B), so that inputs in these dimensions help produce future commands (FIG. 16C). This accords with the finding that motor cortex responses to feedback are initially in the decoder null space before transitioning to neural activity that issues corrective commands 24. Broadly, the results provide a feedback control perspective for how invariant dynamics enable the brain to issue commands across diverse behaviors.

There is almost surely a limitation to the behaviors that particular neural population dynamics are useful for. Motor cortex population activity occupies orthogonal dimensions and shows a markedly different influence on muscle activation during walking and trained forclimb movement, and follows different dynamics for reach and grasp movements. Notably, the finding of decaying dynamics for BMI control contrasts with rotational dynamics observed during natural arm movement. This can be because controlling the BMI relied more on feedback control than a well-trained physical movement, because controlling the BMI did not require the temporal structure of commands needed to control muscles for movement, and/or because controlling the BMI did not involve proprioceptive feedback of physical movement. One intuition would be that behaviors which need particular temporal frequencies of commands elicit neural dynamics that produce those frequencies in their activity transitions. Recent theoretical work shows that cortico-basal ganglia-thalamic loops can switch between different cortical dynamics useful for different temporal patterns of commands.

The use of invariant dynamics to issue commands has implications for how the brain learns new behavior, enabling the brain to leverage pre-existing dynamics for initial learning and to develop new dynamics through gradual reinforcement. This learning that modifies dynamics relies on plasticity in cortico-basal ganglia circuits and permits the brain to reliably access a particular neural activity pattern for a given command and movement, even if the same neural activity pattern is not used to issue the same command across different movements.

The results suggest that modeling invariant dynamics informs the design of new neuroprosthetics that can generalize commands to new behaviors and classify entire movement trajectories. As new behaviors are performed, distinct neural activity patterns can be used to issue the same command, but that invariant dynamics can predict and thus recognize these distinct neural patterns as signal for the BMI rather than noise. In addition, the results inform the design of rehabilitative therapies to restore dynamics following brain injury or stroke to recover movement.

Overall, this study put the output of a neural population into focus, revealing how rules for neural activity transitions are used to issue commands and produce different movements. This was achieved by studying the brain as it skillfully controlled the very neural population activity recorded. BMI, especially combined with technical advances in measuring, modeling, and manipulating activity from defined populations, provides a powerful technique to test emerging hypotheses about how neural circuits generate activity to control behavior.

FIGS. 11A-11F illustrate the BMI used to study neural population control of movement.
(A) Schematic of the BMI system. Subjects generate population activity patterns to control a cursor to hit visual targets under visual feedback. Population activity (Monkey G [J]: 35-151 [20-21] units from motor cortex) is transformed through a linear decoder K into two-dimensional commands (orange arrows) which update the two-dimensional velocity of the cursor.
(B) Before each experiment, the decoder parameters were calibrated in two steps. First, the decoder parameters were initialized based on neural activity recorded while subjects passively watched the cursor move through the tasks. Second, the parameters were continuously adapted as subjects controlled the BMI in closed-loop until performance reached an expected threshold based on previous performance. Then the decoder's parameters were fixed, and the experimental session began.
(C) Single trials from the center-out and obstacle-avoidance task in a Monkey G session.
(D) Average time to reach target on each session. Monkey G [J] took 2.91 [2.04] sec for the center-out task and 3.50 [3.48] sec for the obstacle-avoidance task, on average.
(E) Example of the same command (black arrow) being issued during single trials of different conditions (labelled with the number of the target achieved (0-7) and with ‘co’ for the center-out task or ‘cw’/‘ccw’ for clockwise/counter-clockwise movements around the obstacle in the obstacle-avoidance task). The example command was in the −45 degree direction and the smallest magnitude bin of analysis (see STAR Methods—“Command discretization for analysis”).
(F) Left: The average commands occurring before and after the example command are plotted for each condition (the command subtrajectory from −500 ms to 500 ms). Right: The average positions occurring before and after the example command are plotted for each condition (the position subtrajectory from −500 ms to 500 ms). The position subtrajectories are centered and superimposed at the occurrence of the example command to highlight subtrajectory differences. See FIGS. 17A-17E for quantification of differences in command subtrajectories across conditions.FIGS. 12A-12D illustrate using the BMI to test whether the brain uses invariant dynamics to control different movements.
(A) Illustration of invariant dynamics governing how population activity xₜ transitions in time to xₜ₊₁. Dynamics that are invariant across different movements can be visualized as a flow field in neural activity space, where the arrows depicting flow are the same for different movements.
(B) Knowledge of the BMI transformation from neural activity to command enables identifying the component of neural activity that drives the current command and the component with no effect on the current command. An illustrative decoder defines the command at time t as the difference between two neurons' instantaneous activity x₂(t)−x₁(t), symbolized with orange arrows (top right) indicating the command's magnitude and sign. The component of neural activity in the “decoder space” determines the command, while the component in the “decoder null space” has no effect on the command. The decoder null space implies redundancy, as the space of activity patterns with a given coordinate in the decoder space (given by the dotted-orange lines) issues the same command (such as the two patterns given by the white and black square that issue the same upward command).
(C) A trajectory of commands (orange arrows) produces one whole movement. Movement 1 (blue) and 2 (green) illustrate movements driven by the same commands in different temporal orders. In some embodiments, these movements comprise different transitions through neural activity space. Many different neural trajectories would issue the appropriate commands to produce a given movement.
(D) Illustration of neural activity trajectories that follow the transitions given by invariant dynamics h in order to issue the commands for movement. This illustration shows how population dynamics that are invariant across movement are able to control different movements. See FIG. 19D for how another example of invariant dynamics (decaying dynamics) can control different movements.

FIGS. 13A-13E illustrate that the same command is issued by different neural activity patterns in different movements.
(A) If neural activity follows invariant dynamics to control movement, systematically different activity patterns (blue, green dots) are used to issue the same command (orange upward arrow) in different movement conditions. These patterns deviate from the condition-pooled average activity pattern that issues the command (black dot).
(B) Left: An example neuron's average firing rate (colored dots) for the example command and conditions from FIG. 11F, as well as the condition-pooled average activity (dotted black line labeled “condition-pool”). The condition-shuffled distributions of average activity are shown with gray boxplots labeled “shuffle” indicating the 2.5^{th}, 25^{th}, 50^{th}, 75^{th}, and 97.5^{th} percentiles (see STAR methods—“Matching the condition-pooled distribution” and “Behavior-preserving shuffle of activity”). The neuron's condition-specific distance (e.g., the average absolute difference between condition-specific activity and condition-pooled activity) exceeded shuffle distance (p<0.05) for 62.5% of this example's conditions, as indicated by an asterisk on 5/9 conditions. For more detail, see STAR methods—“Analysis of activity issuing a given command.” Aggregating over all (command, condition, neuron) tuples, the condition-specific distance was significantly greater than shuffle distance: Monkey G [J]: p-value <0.001 for 9/9 [4/4] sessions, p-value <0.001 pooled over sessions. Condition-specific distance averaged over all (command, condition, neuron) tuples=1.167 Hz [1.235 Hz]; mean (95^{th} percentile) of shuffle distance distribution=1.004 (1.010) Hz [0.745 (0.757) Hz]. Right: Same example command and conditions as Left, except showing the condition-specific population distance (colored dots), e.g., the distance between the average population activity vector for a specific condition and the average vector pooling across conditions. This population distance was normalized, displayed as the “factor” of increase relative to the mean of the condition-shuffled distribution of distances. For display, the condition-shuffled distribution of distances was also normalized to its mean and shown with gray boxplots labeled “shuffle” indicating the 0^{th}, 25^{th}, 50^{th}, 75^{th}, and 95^{th} percentiles. The dashed line at 1.0 indicates the shuffle mean distance when normalized to itself. The condition-specific population distance was significantly greater (p<0.05) than the shuffle distances for 78% of the example (command, condition) tuples, as indicated with asterisks on 7/9 tuples. See FIG. 18A for a visualization of the condition-specific population distance for all (command, condition) tuples in this example session.
(C) The distribution of normalized population distances across (command, condition) tuples is displayed for each session. Colored ticks indicate distances in (B) right. See FIG. 18BC to see additional distance distributions, including for single neurons, with different normalization, and for only the (command, condition) tuples that significantly deviate from shuffle.
(D) Normalized population distance averaged across (command, condition) tuples (Monkey G [J]: n=9 [4] sessions). Bars indicate the average across sessions. Aggregating over all (command, condition) tuples, the condition-specific population distance was significantly greater than the shuffle distances: Monkey G [J]: p-value <0.001 for 9/9 [4/4] sessions, p-value <0.001 for pooled over sessions. Normalized condition-specific distance averaged over all (command, condition) tuples=1.222 [1.724]; mean (95^{th} percentile) of shuffle distance distribution=1.0 (1.007), [1.0 (1.019)].
(E) Left: Fraction of (command, condition) tuples with condition-specific distance significantly greater than shuffle distance (Monkey G [J]: n=9 [4] sessions). Middle: Fraction of commands with condition-specific distances significantly greater than shuffle distance, aggregating over conditions (Monkey G [J]: n=9 [4] sessions). Right: Fraction of neurons with condition-specific distance significantly greater than shuffle distance (Monkey G [J]: n=9 [4] sessions), aggregating over all (command, condition) tuples. Throughout (E), the dashed line indicates chance level, which is the fraction equal to 0.05 significantly deviating from the shuffle distribution of distance. See FIGS. 22E-22H for the relationship between condition-specific distance and the structure of behavior across pairs of conditions.

FIGS. 14A-14G illustrate invariant dynamics predict the different neural activity patterns used to issue the same command.

FIG. 14A illustrates whether a linear model of invariant dynamics can predict the different activity patterns (cyan-outlined dots) that issue a given command (orange arrow). Specifically, neural activity given the command it issues, the previous activity pattern (colored rings), and knowledge of the BMI decoder (see STAR methods—“Invariant dynamics model predictions”, “Predicting activity issuing a given command”). Also see FIGS. 22A-22K on predicting structure of activity across pairs of conditions.

FIG. 14B illustrates whether invariant dynamics generalize their predictions to situations in which the invariant dynamics model had no training data. Sec FIGS. 20A-201 for more extensive tests of invariant dynamics generalization. Left: illustrates whether the invariant dynamics model can predict neural activity for a given command that was left-out of training the model (illustrated in magenta). Right: illustrates whether the invariant dynamics model can predict neural activity for a given command and condition if all neural activity in that condition is left-out of training the model (illustrated in purple).
(C) R² of models predicting neural activity given the command it issues and previous activity (Monkey G [J]: n=9 [4] sessions). See FIGS. 19A-19F for properties of the models (e.g., eigenvalues and dimensionality) and the residuals. The models included: 1) the dynamics model that is trained using a complete dataset and that predicts held-out test data (cyan, labeled “full”), 2) the dynamics model that is trained with a command left out and that predicts the left-out command (magenta, labeled “command left-out”), 3) the dynamics model that is trained with a condition left out and that predicts the left-out condition (purple, labeled “condition left-out”), 4) the dynamics model that is trained using a shuffled complete dataset (see STAR methods—“Behavior-preserving shuffle of activity”) and that predicts held-out, unshuffled test data (black, labeled “shuffle”), and 5) a model trained using a complete dataset and that predicts held-out test neural activity just given the command but not given previous neural activity (gray, labeled “command only”). For elaboration, see STAR methods—“Invariant dynamics models.” To evaluate the significance of each model R² relative to shuffle dynamics, a distribution of shuffle dynamics R² values was generated by computing one R² value per shuffled dataset. The value of the “shuffle” bar is the 95^{th} percentile of the shuffle distribution. The main panel shows the models' R² normalized by the 95^{th} percentile of shuffle dynamics R², while the inset shows the raw R². Full dynamics, command left-out dynamics, and condition left-out dynamics all predict neural activity significantly better than shuffle dynamics. Shuffle dynamics: Monkey G [J]: mean (95^{th} percentile, displayed) R²=0.130 (0.130) [0.196 (0.196)]. Full dynamics: Monkey G [J]: p-value <0.001 for 9/9 [4/4] sessions, p-value <0.001 for sessions pooled, mean R²=0.167 [0.252]. Command left-out dynamics: Monkey G [J]: p-value <0.001 for 9/9 [4/4] sessions, p-value <0.001 for sessions pooled, mean R²=0.163 [0.243]. Condition left-out dynamics: Monkey G [J]: p-value <0.001 for 9/9 [4/4] sessions, p-value <0.001 for sessions pooled, mean R²=0.163 [0.240]. FIGS. 21A-21F show that invariant dynamics models predict more than just encoding of behavior variables, and that a state-of −art non-linear model of invariant dynamics does not improve upon the linear model.
(D) Left. Average activity for the example neuron, command, and conditions from FIG. 13B, left.

Right. Prediction of the condition-specific average activity for the example neuron by the full dynamics model (stars), the shuffle dynamics model (black boxplot distribution), and the model predicting neural activity only using the command (gray triangle). To evaluate whether the full dynamics model predictions were significant (see STAR methods—“Invariant dynamics model predictions”, “Comparing invariant dynamics to shuffle”), the error is computed as the distance between the condition-specific average activity for a given command and the model prediction for this quantity, and compared the error of the full dynamics prediction to the distribution of errors from shuffle dynamics predictions. For the example command and neuron, a fraction of 0.889 conditions are significantly predicted as indicated by the black asterisks. Aggregating over all (command, condition, neuron) tuples, the full dynamics model predicted individual neuron activity better than shuffle dynamics. Shuffle dynamics: Monkey G [J]: mean (5^{th} percentile) of error: 1.359 (1.359 Hz) [1.455 (1.454)]. Full dynamics: Monkey G [J]: p-value <0.001 for 9/9 [4/4] sessions, p-value <0.001 for pooled sessions, error averaged over all (command, condition, neuron) tuples: 1.232 Hz [1.182 Hz].
(E) Left. Average population activity for the example command and conditions from FIG. 13B right, visualized along the activity dimension that captured the most neural activity variance (the first principal component, labeled “PC1”, from principal components analysis applied to condition-specific average population activity). Right. Prediction of the condition-specific average population activity along the same PC1 by the full dynamics model (stars), the shuffle dynamics model (black boxplot distribution), and the model predicting neural activity only using the command (gray triangle). For the example command, all conditions (e.g., fraction of 1.0) are significantly predicted as indicated by the black asterisks (significance was calculated using full population activity, not just PC1). Aggregating over all (command, condition) tuples, the full dynamics model predicted population activity better than shuffle dynamics, based on comparison of the error (distance between true and predicted average population activity for a given command and condition) normalized by shuffle mean error. Shuffle dynamics: Monkey G [J]: mean (5^{th} percentile) of normalized error=1.0 (0.99), [1.0, (0.99)]. Full dynamics: Monkey G [J]: p-value <0.001 for 9/9 [4/4] sessions, p-value <0.001 for pooled sessions, mean normalized error=0.883 [0.809].
(F) the condition-specific component of neural activity is isolated for a given command and condition as: condition-specific average population activity minus the prediction of that quantity based on the command alone. (For detail, See STAR methods—“Invariant dynamics model predictions”, “Predicting condition-specific component”.) The bar plot shows the model R² from predicting the condition-specific component of neural activity for a given command, comparing the full dynamics model (dark gray bar and filled dots) with the mean of the shuffle dynamics model (light bar and empty dots) (Monkey G [J]: n=9 [4] sessions). The full dynamics model predicted significantly more variance of the condition-specific component of neural activity than shuffle dynamics: Monkey G [J]: p-value <0.001 for 9/9 [4/4] sessions, p-value <0.001 for pooled sessions, (mean R² for dynamics model 0.226 [0.330], mean (95^{th} percentile) of R² of shuffled dynamics −0.006 (−0.005) [−0.016 (−0.014)].
(G) Analyses of how well neural activity is predicted for individual commands and conditions. (For detail, see STAR methods—“Invariant dynamics model predictions”, “Predicting condition-specific activity”.) Left. Fraction of (command, condition) tuples where full dynamics predicts condition-specific average population activity significantly better than shuffle dynamics (Monkey G [J]: n=9 [4] sessions). Center. Fraction of commands, aggregated over all conditions, where full dynamics predicts condition-specific average population activity significantly better than shuffle dynamics (Monkey G [J]: n=9 [4] sessions). Right. Fraction of neurons, aggregated over all (command, condition) tuples, where full dynamics predicts the neuron's average activity significantly better than shuffle dynamics (Monkey G [J]: n=9 [4] sessions).

FIGS. 15A-15G illustrate invariant dynamics align with the decoder, propagating neural activity to issue the next command.

FIG. 15A illustrates whether invariant dynamics are used to transition neural activity towards the next command needed for each movement. Given that invariant dynamics predicted the neural activity issuing a given command (colored rings), it is analyzed to determine whether invariant dynamics predict next neural activity (cyan-outlined dots) and next commands (orange symbols). Specifically, the next command by applying the predicted next neural activity through the BMI decoder K (see STAR methods—“Invariant dynamics model predictions”, “Predicting next command”).
(B) Invariant dynamics do not necessarily predict the transition to the next command. It is possible that invariant dynamics only define activity transitions in the neural activity space that is orthogonal to the BMI decoder (pink plane, labeled “decoder null space”). If this were the case, invariant dynamics would predict the next neural activity in the decoder null space, but the predicted transition would not change the next command.
(C) R² of models predicting next neural activity given current neural activity (Monkey G [J]: n=9 [4] sessions) including 1) the dynamics model trained using a complete dataset and that predicts held-out test data (cyan, labeled “full”), 2) the dynamics model trained with an command left out and that predicts for the left-out command (magenta, labeled “command left-out”), 3) the dynamics model trained with a condition left out and that predicts for the left-out condition (purple, labeled “condition left-out”), 4) the dynamics model trained on the component of neural activity in the decoder null space and that predicts held-out test data (pink, labeled “decoder-null”), and 5) the dynamics model trained using a shuffled complete dataset and that predicts held-out unshuffled test data (black, labeled “shuffle”). For elaboration, see STAR methods-“Invariant dynamics models.” The value of the “shuffle” bar is the 95^{th} percentile of the shuffle distribution. The main panel shows the models' R² normalized by the 95^{th} percentile of shuffle dynamics R², while the inset shows the raw R². All models predicted next neural activity significantly better than shuffle dynamics. To evaluate the significance of each model R² relative to shuffle dynamics, a distribution of shuffle dynamics R² values was generated by computing one R² value per shuffled dataset. Shuffle dynamics: Monkey G [J]: mean (95^{th} percentile) R²=0.055 (0.055), [0.051 (0.053)]. Full dynamics: Monkey G [J]: p-value <0.001 for 9/9 [4/4] sessions, p-value <0.001 for sessions pooled, mean R²=0.100 [0.117]. Command left-out dynamics: Monkey G [J]: p-value <0.001 for 9/9 [4/4] sessions, p-value <0.001 for sessions pooled, mean R²=0.099 [0.113]. Condition left-out dynamics: Monkey G [J]: p-value <0.001 for 9/9 [4/4] sessions, p-value <0.001 for sessions pooled, mean R²=0.097 [0.103]. Decoder-null dynamics: Monkey G [J]: p-value <0.001 for 9/9 [4/4] sessions, p-value <0.001 for sessions pooled, mean R²=0.083 [0.085].
(D) Same as (C), except prediction of next command given current neural activity (Monkey G [J]: n=9 [4] sessions). All models except decoder-null dynamics predicted next command significantly better than shuffle dynamics. Shuffle dynamics (black): mean (95^{th} percentile) R² of shuffle=0.264 (0.266) [0.186 (0.188)]. Full dynamics (orange): Monkey G [J]: p-value <0.001 for 9/9 [4/4] sessions, p-value <0.001 for sessions pooled, mean R²=0.315 [0.212]. Command left-out (magenta): Monkey G [J]: p-value <0.001 for 9/9 [4/4] sessions, p-value <0.001 for sessions pooled, mean R²=0.310 [0.211]. Condition left-out (purple): Monkey G [J]: p-value <0.001 for 9/9 [2/4] sessions, p-value <0.05 for 9/9 [3/4] sessions, p-value n.s. for 0/9 [1/4] sessions, p-value <0.001 for sessions pooled, mean R²=0.305 [0.193]. Decoder-null dynamics (pink): Monkey G [J]: p-value n.s. for 9/9 [4/4] sessions, p-value n.s. for sessions pooled, mean R²=0.00 [0.00].
(E) Analyses of how well the next command is predicted for individual (command, condition) tuples. The error is computed between the true and predicted average next command for a given current command and condition, and compared the error of the full dynamics prediction and shuffle dynamics prediction. Aggregating over all (command, condition) tuples, the full dynamics model predicted condition-specific next command better than shuffle dynamics. Shuffle dynamics: Monkey G [J]: mean (5^{th} percentile) error of predicted next command=5.40 (5.38), [9.305 (9.240)]. Full dynamics: Monkey G [J]: p-value <0.001 for 9/9 [4/4] sessions, p-value <0.001 for pooled sessions, mean error of predicted next command=3.956 [7.324]. Left. Fraction of (command, condition) tuples where full dynamics predicts the next command significantly better than shuffle dynamics (Monkey G [J]: n=9 [4] sessions). Right. Fraction of commands, aggregated over all conditions, where full dynamics predicts the next condition-specific command significantly better than shuffle dynamics (Monkey G [J]: n=9 [4] sessions).
(F) Visualization of the command angle (left) (e.g., the direction that the command points) for the example command and conditions (right) from FIG. 13B. For each example condition (each row), visualization shows the average current command angle (first column), the average next command angle (second column), and the prediction of the average next command angle by the full dynamics model (third column).
(G) For each (command, condition) tuple, prediction of the angle between the next command and the condition-pooled average next command. Left. Fraction of (command, condition) tuples for which the sign of the angle is accurately predicted (positive=turn counterclockwise, negative=turn clockwise). Full dynamics predictions are significantly more accurate than shuffle dynamics (Monkey G [J]: p-value <0.001 for 9/9 [4/4] sessions, p-value <0.001 for pooled sessions, mean fraction correct=0.708 [0.617]. Shuffle dynamics mean (95^{th} percentile)=0.535 (0.541) [0.473 (0.480)]. Right. Error in predicted angle. Full dynamics predictions are significantly more accurate than shuffle dynamics (Monkey G [J]: p-value <0.001 for 9/9 [4/4] sessions, p-value <0.001 for pooled sessions, mean angular error=19.503 (9.608). Shuffle dynamics mean (5^{th} percentile)=26.564 (26.415) [12.779 (12.636)].

FIG. 16 illustrates an OFC model reveals that invariant dynamics reduce the input that a neural population needs to issue commands based on feedback.
(A) An optimal feedback control model in which the brain processes ongoing task state and population activity into an input to the neural population. The neural population activity that issues commands for movement is driven by three terms: invariant dynamics observed experimentally, input from an optimal feedback controller, and noise.
(B) Three simulated trials for each condition (center-out (co), counter-clockwise (ccw), and clockwise (cw) movements to 8 targets resulting in 24 conditions). Top: Full Dynamics Model that uses invariant dynamics fit on experimental data. Bottom: No Dynamics Model that uses dynamics matrix A set to 0.
(C) Input magnitude. Data for each session is shown as a percentage of the No Dynamics Model (Monkey G [J]: n=9 [4] sessions). In some embodiments, the neural population takes in significantly less input to control movement under the Full Dynamics Model (cyan ‘D’) as compared to the No Dynamics Model (black ‘ND’). The models' un-normalized data were pooled across sessions and compared with a linear mixed effect (LME) model between input magnitude and model category with session modeled as random effect (Monkey G [J]: N=432 [192], z=10.49 [5.20], p-value=9.67e-26 [1.92e-7]). Individual sessions were analyzed with a Wilcoxon signed-rank test that paired condition across the models (Monkey G [J]: p-value<0.05 for 9/9 [4/4] sessions).
(D) Same as (C) but for Decoder-null Dynamics (e.g., model of invariant dynamics fit on experimental data that is restricted to the decoder-null space). There was no significant difference in input magnitude between Decoder-null Dynamics (pink ‘D’) and No Dynamics (black ‘ND’) when pooling across sessions (Monkey G [J]: N=432 [192], z=0.002 [−0.003], p-value=9.98e-1 [9.90e-1]) and on individual sessions (Monkey G [J]: p-value<0.05 for 0/9 [0/4] sessions).
(E) The same command is issued across conditions in both the Full Dynamics Model and No Dynamics Model. Average cursor position subtrajectories are shown locked to the occurrence of the example command across example conditions.
(F) Condition-specific population distance (e.g., the distance between 1) population activity issuing the example command in a specific condition and 2) population activity issuing the example command averaged across conditions). Distances were normalized by the mean of the shuffle distribution (gray boxplots showing mean, 0^{th} percentile, 25^{th}, 75^{th}, and 95^{th} percentile). Left: data from Full Dynamics Model. Right: data from the No Dynamics Model. Asterisk indicates distance is greater than shuffle (p-value<0.05).
(G) Same as (F), but each data point is an individual session that pools over (command, condition) tuples (Monkey G [J]: n=9 [4] sessions). Population distances for the Full Dynamics Model were greater than shuffle. Data was pooled over sessions using a LME with session modeled as random effect (Monkey G [J]: N=4906 [2408], z=−23.09 [z=−16.68], p-value=6.37e-118 [1.77e-62]), and individual sessions were analyzed with a Mann-Whitney U test (p-value<0.05 for Monkey G [J] on 9/9 [4/4] sessions). No difference was detected in population distances between the No Dynamics Model and shuffle when pooling across sessions (Monkey G [J]: N=4334 [2188], z=0.168 [0.462], p-value=8.66e-1 [6.44e-1]) and on individual sessions (p-value<0.05 for Monkey G (J) on 0/9 (0/4) sessions).
(H) Same as (G), but for the Decoder-null Dynamics Model (pink ‘D’). No difference was detected in population distances between the Decoder-null Dynamics Model and shuffle when pooling across sessions (Monkey G [J]: N=4488 [2252], z=−0.932 [−1.490], p-value=3.51e-1 [1.36e-1]) and on individual sessions (p-value<0.05 for Monkey G (J) on 0/9 (0/4) sessions). Also, no difference was detected in population distances between the No Dynamics Model and shuffle when pooling across sessions (Monkey G [J]: N=4482 [2250], z=0.611 [0.449], p-value=5.41e-1 [6.54e-1]) and on individual sessions (p-value<0.05 for Monkey G (J) on 0/9 (0/4) sessions).

All training, surgery, and experimental procedures were conducted in accordance with the NIH Guide for the Care and Use of Laboratory Animals and were approved by the University of California, Berkeley Institutional Animal Care and Use Committee (IACUC). Two adult male rhesus macaque monkeys (7 years old, monkey G and 10 years old, monkey J) (Macaca mulatta, RRID: NCBITaxon: 9544) were used as subjects in this study. Prior to this study, Monkeys G and J were trained at arm reaching tasks and spike-based 2D neuroprosthetic cursor tasks for 1.5 years. All animals were housed in pairs.

For electrophysiology and experimental setup, two male rhesus macaques were bilaterally, chronically implanted with 16×8 arrays of Teflon-coated tungsten microwire electrodes (35 mm in diameter, 500 mm separation between microwires, 6.5 mm length, Innovative Neurophysiology, Durham, NC) in the upper arm area of primary motor cortex (M1) and posterior dorsal premotor cortex (PMd). Localization of target areas was performed using stereotactic coordinates from a neuroanatomical atlas of the rhesus brain. Implant depth was chosen to target layer 5 pyramidal tract neurons and was typically 2.5-3 mm, guided by stereotactic coordinates.

During behavioral sessions, neural activity was recorded, filtered, and thresholded using the 128-channel Multichannel Acquisition Processor (Plexon, Inc., Dallas, TX) (Monkey J) or the 256-channel Omniplex D Neural Acquisition System (Plexon, Inc.) (Monkey G). Channel thresholds were manually set at the beginning of each session based on 1-2 min of neural activity recorded as the animal sat quietly (e.g., not performing a behavioral task). Single-unit and multi-unit activity were sorted online after setting channel thresholds. Decoder units were manually selected based on a combination of waveform amplitude, variance, and stability over time.

For neuroprosthetic decoding, subjects' neural activity controlled a two-dimensional (2D) neuroprosthetic cursor in real-time to perform center-out and obstacle-avoidance tasks. The neuroprosthetic decoder consists of two models:
1) A cursor dynamics model capturing the physics of the cursor's position and velocity.
2) A neural observation model capturing the statistical relationship between neural activity and the cursor.

In some embodiments, the neuroprosthetic decoder combines the models according to predetermined conditions to estimate the subjects' intent for the cursor and to correspondingly update the cursor.

### Decoder Algorithm and Calibration—Monkey G

Monkey G used a velocity Kalman filter (KF) 96.97 that uses the following models for cursor state cₜ and observed neural activity xₜ:

$$\begin{matrix}{c}_{t} = A {c}_{t - 1} + {w}_{t} , {w}_{t} ∼ N ( 0 , W ) \\ {x}_{t} = C {c}_{t} + {q}_{t} , {q}_{t} ∼ N ( 0 , Q )\end{matrix}$$

In the cursor dynamics model, the cursor state cₜ∈R⁵ was a 5-by-1 vector [posₓ, pox_{y}velₓ, vel_{y}, 1]^{T}, A∈R^{5×5} captures the physics of cursor position and velocity, and wₜ is additive Gaussian noise with covariance W∈R^{5×5} capturing cursor state variance that is not explained by A.

In the neural observation model, neural observation xₜ∈R^{N} was a vector corresponding to spike counts from N units binned at 10 Hz, or 100 ms bins. C models a linear relationship between the subjects' neural activity and intended cursor state. The decoder only modeled the statistical relationship between neural activity and intended cursor velocity, so only the columns corresponding to cursor state velocity and the offset (columns 3-5) in C were non-zero. Q is additive Gaussian noise capturing variation in neural activity that is not explained by Ccₜ. For Monkey G, 35-151 units were used in the decoder (median 48 units).

In summary, the KF is parameterized by matrices {A∈R^{5×5}, W∈R^{5×5}, C∈R^{N×5}, Q∈R^{N×N}}. The KF equations used to update the cursor based on observations of neural activity are defined as in ⁹⁷.

The KF parameters were defined as follows. For the cursor dynamics model, the A and W matrices were fixed as in previous studies 98. Specifically, they were:

$$A = [ \begin{matrix}1 & 0 & 0.1 & 0 & 0 \\ 0 & 1 & 0 & 0.1 & 0 \\ 0 & 0 & 0.8 & 0 & 0 \\ 0 & 0 & 0 & 0.8 & 0 \\ 0 & 0 & 0 & 0 & 1\end{matrix} ] , W = [ \begin{matrix}0 & 0 & 0 & 0 & 0 \\ 0 & 0 & 0 & 0 & 0 \\ 0 & 0 & 7 & 0 & 0 \\ 0 & 0 & 0 & 7 & 0 \\ 0 & 0 & 0 & 0 & 0\end{matrix} ]$$

where units of cursor position were in cm and cursor velocity in cm/sec.

For the neural observation model, the C and Q matrices were initialized from neural and cursor kinematic data collected at the beginning of each experimental session while Monkey G observed 2D cursor movements that moved through either a center-out task or obstacle avoidance task. Maximum likelihood methods were used to fit C and Q.

Next, Monkey G performed a “calibration block” where he performed the center-out or obstacle-avoidance task movements as the newly initialized decoder parameters were continuously calibrated/adapted online (“closed-loop decoder adaptation”, or CLDA). This calibration block was performed in order to arrive at parameters that would enable excellent neuroprosthetic performance. At predetermined time intervals, decoder matrices C and Q were adapted using the recursive maximum likelihood CLDA algorithm. Half-life values, defining how quickly C and Q can adapt, were typically 300 sec, and adaptation blocks were performed with a weak, linearly decreasing “assist” (re-defining cₜ as a weighted linear combination of user-generated cₜ and optimal cₜ to drive the cursor to the target). Typical assist values at the start of the block were 90% user-generated, 10% optimal and decayed to 100% user-generated, 0% optimal over the course of the block. Following CLDA, decoder parameters were fixed. Then the experiment proceeded with Monkey G performing the center-out and obstacle-avoidance tasks.

### Decoder Algorithm—Monkey J

Monkey J used a velocity Point Process Filter (PPF). The PPF uses the same cursor dynamics model for cursor state cₜ as the KF above, but uses a different neural observations model (a Point Process model rather than a Gaussian model) for the spiking Sₜ^{1:N} of each of N neurons:

$$\begin{matrix}{c}_{t} = A {c}_{t - 1} + {w}_{t} , {w}_{t} ∼ N ( 0 , W ) \\ p ( {S}_{t}^{1 : N} \vert {v}_{t} ) = {\prod }_{j = 1}^{N} {( {λ}_{j} ( t \vert {v}_{t} , {ϕ}^{j} ) Δ )}^{{S}_{t}^{j}} exp ( - {λ}_{j} ( t \vert {v}_{t} , {ϕ}^{j} ) Δ )\end{matrix}$$

In the neural observations model, neural observation Sₜ^{j} is the j^{th} neuron's spiking activity, equal to 1 or 0 depending on whether the j^{th} neuron spikes in the interval (t, t+Δ). Δt=5 ms bins are used since consecutive spikes rarely occurred within 5 ms of each other. For Monkey J, 20 or 21 units were used in the decoder (median 20 units). The probability distribution over spiking

$$p ( {S}_{t}^{1 : N} \vert {v}_{t} )$$

was a point process win λⱼ(t|vₜ, ϕ^{j}) as the j^{th} neuron's instantaneous firing rate at time t. λⱼ(t|vₜ, ϕ^{j}) depended on the intended cursor velocity vₜ∈R² in the two dimensional workspace and the parameters ϕ^{j} for how neuron j encodes velocity. λⱼ(t|vₜ, ϕ^{j}) was modeled as a log-linear function of velocity:

$${λ}_{j} ( t \vert {v}_{t} , {ϕ}^{j} ) = exp ( {β}_{j} + {α}_{j}^{T} {v}_{t} )$$

where ϕ^{j} parameters consist of αⱼ∈R², βⱼ∈R¹.

In summary, the PPF is parameterized by {A∈R^{5×5}, W∈R^{5×5}, ϕ^{1:N}}. The PPF equations used to update the cursor based on observations of neural activity are defined as in 48.

The PPF parameters were defined as follows. For the cursor dynamics model, the A and W matrices are defined as:

$$A = [ \begin{matrix}1 & 0 & 0.005 & 0 & 0 \\ 0 & 1 & 0 & 0.005 & 0 \\ 0 & 0 & 0.989 & 0 & 0 \\ 0 & 0 & 0 & 0.989 & 0 \\ 0 & 0 & 0 & 0 & 1\end{matrix} ] , W = [ \begin{matrix}0 & 0 & 0 & 0 & 0 \\ 0 & 0 & 0 & 0 & 0 \\ 0 & 0 & 3.7 \times {10}^{- 5} & 0 & 0 \\ 0 & 0 & 0 & 3.7 \times {10}^{- 5} & 0 \\ 0 & 0 & 0 & 0 & 0\end{matrix} ]$$

where units of cursor position were in m and cursor velocity in m/sec.

For the neural observations model, parameters ϕ^{1:N} were initialized from neural and cursor kinematic data collected at the beginning of each experimental session while Monkey J observed 2D cursor movements that moved through a center-out task. Decoder parameters were adapted using CLDA and optimal feedback control intention estimation as outlined in. Following CLDA, decoder parameters were fixed. Then the experiment proceeded with Monkey J performing the center-out and obstacle-avoidance tasks.

The “command” is defined for the BMI as the direct influence of subjects' neural activity xₜ (binned at 100 ms) on the cursor. Concretely, in both decoders, the command was a linear transformation of neural activity represented as Kxₜ which updated the cursor velocity.

### Command Definition—Monkey G

For Monkey G, the update to the cursor state cₜ due to cursor dynamics and neural observation xₜ can be written as:

$${c}_{t} = {F}_{t} {c}_{t - 1} + {K}_{t} {x}_{t}$$

where FₜCₜ₋₁ is the update in cursor state due to the cursor dynamics process and Kₜxₜ is defined as the command: the update in cursor state due to the current neural observation. Kₜ∈R^{5×n} is the Kalman Gain matrix and Fₜ=(I−KₜC) A. In practice Kₜ converges to its steady-state form K within a matter of seconds, and thus Fₜ converges to F=(I−KC) A, so the above expression can be represented in its steady state form:

$${c}_{t} = F {c}_{t - 1} + K {x}_{t}$$

In one implementation, the structure of K is such that neural activity xₜ directly updates cursor velocity, and velocity integrates to update position. The following technical note explains the structure of K. Due to the form of the A, W matrices, Rank(K)=2. In addition, decoder adaptation imposed the constraint that the intermediate matrix C^{T}Q⁻¹C was of the form aI, where a=mean (diag(C^{T}Q⁻¹C)). Due to this constraint, the rows of K that update the position of the cursor are equal to the rows of K that update the velocity multiplied by the update timestep: K(1:2, :)=K(3:4, :)*dt¹⁰⁰ (see independent velocity control in the reference). Given this structure of K, neural activity's contribution to cursor position is the simple integration of neural activity's contribution to velocity over one timestep.

In summary, since Kxₜ reflects the direct effect of the motor cortex units on the velocity of the cursor, the velocity components of Kxₜ can be also referred to as the “command”. The neural spike counts binned at 100 ms, which were used online to drive cursor movements with no additional pre-processing, are analyzed.

### Command Definition—Monkey J

For Monkey J the cursor state updates in time as:

$$\begin{matrix}{c}_{t} = {f}_{t} ( {c}_{t - 1} ) + {K}_{t} {x}_{t} \\ where \\ {f}_{t} ( {c}_{t - 1} ) = ( A {c}_{t - 1} - {K}_{t} {e}^{{cAc}_{t - 1}} Δ ) , {K}_{t} = {P}_{t} C\end{matrix}$$

Here fₜ(cₜ₋₁) is the cursor dynamics process and Kₜxₜ is the neural command. Pₜ∈R^{5×5} is the estimate of cursor state covariance, and C∈R^{5×N} captures how neural activity encodes velocity as a matrix where each column is composed of

$${[ 0 , 0 , {α}_{j}^{xvel} , {α}_{j}^{yvel} , {β}_{j} ]}^{T}$$

for the j^{th} unit.

The command for analysis in this study is defined as Kₑₛₜxₜ, where Kₑₛₜ is a time-invariant matrix that almost perfectly approximates Kₜ. While the PPF's Kₜ does not necessarily converge in the same way it does in the KF, for all four analyzed sessions, neural activity mapped through Kₑₛₜ∈R^{2×N} can account for 99.6, 99.6, 99.5, and 99.8 percent of the variance of the command respectively (Kₜxₜ≈Kₑₛₜxₜ). In addition, due to the accuracy of this linear approximation, Monkey J's timescale of neural activity and commands are matched to that of Monkey G. In order to match timescales across the two animals (Monkey G: 100 ms updates, Monkey J: 5 ms updates), Monkey J's commands were aggregated into 100 ms bins by summing Kₑₛₜxₜ over 20 consecutive 5 ms bins to yield the aggregated command over 100 ms. Correspondingly, Monkey J's neural activity was also summed into 100 ms bins by summing xₜ over 20 consecutive 5 ms bins.

With neuroprosthetic tasks, subjects performed movements in a two-dimensional workspace (Monkey J: 24 cm×24 cm, Monkey G: 50 cm×28 cm) for two neuroprosthetic tasks: a center-out task and an obstacle-avoidance task. The movement “condition” is defined as the task performed (“co”=center-out task, “cw”/“ccw”=clockwise/counterclockwise movement around the obstacle in the obstacle-avoidance task) and the target achieved (numbered 0 through 7). Thus, there were up to 24 different conditions possible (8 center-out conditions, 8 clockwise obstacle-avoidance conditions, 8 counterclockwise obstacle-avoidance conditions). In practice, subjects mostly circumvented the obstacles for a given target location consistently in a clockwise or counterclockwise manner (as illustrated in FIG. 11C right) resulting in an average of 16-17 conditions per session.

In some embodiments, to be able to perform a center-out task, subjects hold their cursor within a center target (Monkey J: radius=1.2 cm, Monkey G: radius=1.7 cm) for a specified period of time (Monkey J: hold=0.25 sec, Monkey G: hold=0.2 sec) before a go cuc signaled the subjects to move their cursor to one of eight peripheral targets uniformly spaced around a circle. Each target was equidistant from the center starting target (Monkey J: distance=6.5 cm, Monkey G: distance=10 cm). Subjects then had to position their cursor within the peripheral target (Monkey J: target radius=1.2 cm, Monkey G: target radius=1.7 cm) for a specified period to time (Monkey J: hold=0.25, Monkey G: hold=0.2 sec). Failure to acquire the target within a specified window (Monkey J: 3-10 sec, Monkey G: 10 sec) or to hold the cursor within the target for the duration of the hold period resulted in an error. Following successful completion of a target, a juice reward was delivered. Monkey J was trained to move his cursor back to the center target to initiate a new trial, and Monkey G's cursor was automatically reset to the center target to initiate a new trial.

Monkey G performed an obstacle-avoidance task with a very similar structure to the center-out task. The only difference was that a square obstacle (side length 2 or 3 cm) would appear in the workspace centered exactly in the middle of the straight line connecting the center target position and peripheral target position. If the cursor entered the obstacle, the trial would end in an error, and the trial was repeated.

In some embodiments, Monkey J's obstacle-avoidance task involves a point-to-point movement between an initial (not necessarily center) target and another target. On arrival at the initial target, an ellipsoid obstacle appeared on the screen. If the cursor entered the obstacle at any time during the movement to the peripheral target, an error resulted, and the trial was repeated. Target positions and obstacle sizes and positions were selected to vary the amount of obstruction, radius of curvature around the obstacles, and spatial locations of targets. Trials were constructed to include the following conditions: no obstruction, partial obstruction with low-curvature, full obstruction with a long distance between targets, and full obstruction with a short distance between targets thus requiring a high curvature. In this study, only trials that included partial obstruction or full obstruction were analyzed as “obstacle-avoidance” trials.

9 sessions of data from Monkey G and 4 sessions of data from Monkey J are analyzed where on each session, monkeys performed both the center-out and obstacle-avoidance tasks with the same decoder. Only successful trials were analyzed.

In relation to optimal feedback control model and simulation, a model based on optimal feedback control (OFC) is introduced for how the brain can use invariant neural population dynamics to control movement based on feedback. From the perspective of the brain trying to control the BMI, the model is used to study how invariant neural population dynamics affect the brain's control of movement.

Thus, simulations of a model are performed and analyzed in which the brain acts as an optimal linear feedback controller (finite horizon linear quadratic regulator), sending inputs to a neural population so that it performs the center-out and obstacle-avoidance tasks (FIG. 16). In some embodiments, the feedback controller computed suitable inputs given predetermined conditions to the neural population based on the current cursor state and current neural population activity. Specifically, the inputs were computed as the solution of an optimization problem that used knowledge of the target and task, decoder, and the neural population's invariant dynamics. 20 trials are simulated for each of 24 conditions: 8 center-out conditions, 8 clockwise obstacle-avoidance conditions, and 8 counterclockwise obstacle-avoidance conditions. The neural and cursor dynamics processes in the simulation are summarized below:

For neural population dynamics with input, in one simulation, the neural activity of N neurons xₜ∈R^{N} is driven by invariant dynamics A∈R^{N×N} that act on previous activity xₜ₋₁, an activity offset b∈R^{N}, inputs from the feedback controller uₜ₋₁∈R^{N} that are transformed by input matrix B∈R^{N×N}, and noise σₜ₋₁ ∈R^{N}:

$${x}_{t} = {Ax}_{t - 1} + b + {Bu}_{t - 1} + {σ}_{t - 1}$$

The input matrix B was set to be the identity matrix such that each neuron has its own independent input. Each neuron also had its own independent, time-invariant noise (see Noise section below for how the noise level was set).

For notational convenience, an offset term was appended to xₜ:

$$[ \begin{matrix}{x}_{t} \\ 1\end{matrix} ] ∈ {R}^{N + 1} .$$

This enabled incorporating the offset b into the neural dynamics matrix:

$$[ \begin{matrix}{x}_{t} \\ 1\end{matrix} ] = [ \begin{matrix}A & b \\ 0 & 1\end{matrix} ] [ \begin{matrix}{x}_{t - 1} \\ 1\end{matrix} ] + [ \begin{matrix}B \\ 0\end{matrix} ] {u}_{t - 1} + [ \begin{matrix}{σ}_{t - 1} \\ 0\end{matrix} ]$$

In relation to BMI cursor dynamics, the cursor update equations for the simulation matched the steady state cursor update equations in the online BMI experiment (see “Definition of the command for the BMI” above):

$${c}_{t} = F {c}_{t - 1} + {Kx}_{t - 1}$$

As in the experiment, cursor state cₜ∈R^{N}^{_{c}} where N_{c}=5 was a vector consisting of two-dimensional position, velocity, and an offset: [posₓ, pox_{y} velₓ, vel_{y}, 1]^{T}. K∈R^{N}^{_{c}}^{×N} was the decoder's steady-state Kalman gain (Monkey G) or estimated equivalent Kₑₛₜ (Monkey J). F∈R^{N}^{_{c}}^{×N}^{_{c}} was set to the decoder's steady-state cursor dynamics matrix (Monkey G). For Monkey J, F was estimated using the expression for calculating the steady-state cursor dynamics matrix: Fₑₛₜ=(I−KₑₛₜCₑₛₜ)*A_{100 ms}, where I∈R^{N}^{_{c}}^{×N}^{_{c}}, Cₑₛₜ∈R^{N×N}^{_{c}} was set using the α, β velocity encoding parameters from the point process filter (see above): Cₑₛₜ(j, :)=[0 0 0.01*αⱼ(1) 0.01*αⱼ(2) 0.01*βⱼ]. Values in Cₑₛₜ were multiplied by 0.01 to adjust for velocities expressed in units of cm/sec (in the simulation) instead of m/sec (as in PPF). A_{100 ms} was set to the same A used by Monkey G so that the cursor dynamics would be appropriate for 100 ms timesteps:

$${A}_{100 ms} = [ \begin{matrix}1 & 0 & 0.1 & 0 & 0 \\ 0 & 1 & 0 & 0.1 & 0 \\ 0 & 0 & 0.8 & 0 & 0 \\ 0 & 0 & 0 & 0.8 & 0 \\ 0 & 0 & 0 & 0 & 1\end{matrix} ]$$

### Joint Dynamics of Neural Activity and Cursor

The feedback controller sent inputs to the neural population which were optimal considering the task goal, the cursor's current state, the neural population's invariant dynamics, and the neural population's current activity. To solve for the optimal input given all the listed quantities, first, the neural and cursor states are jointly defined. The cursor state cₜ is appended to the neural activity state

$$[ \begin{matrix}{x}_{t} \\ 1\end{matrix} ]$$

to form zₜ∈R^{N+1+N}^{_{c}}:

$${z}_{t} = [ \begin{matrix}{x}_{t} \\ 1 \\ {c}_{t}\end{matrix} ] = [ \begin{matrix}A & b & 0 \\ 0 & 1 & 0 \\ K & 0 & F\end{matrix} ] [ \begin{matrix}{x}_{t - 1} \\ 1 \\ {c}_{t - 1}\end{matrix} ] + [ \begin{matrix}B \\ 0 \\ 0\end{matrix} ] {u}_{t - 1} + [ \begin{matrix}{σ}_{t - 1} \\ 0 \\ 0\end{matrix} ]$$

In words, this expression defines a linear dynamical system where input uₜ₋₁ influences only the neural activity xₜ, xₜ evolves by invariant dynamics A with offset vector b, and xₜ drives cursor cₜ through the BMI decoder K. Finally, noise σₜ₋₁ only influences neural activity xₜ (see Noise section below for how the noise level was set).

For OFC to reach a target, the OFC model computes input uₜ to the neural population such that the activity of the neural population xₜ drives the cursor to achieve the desired final cursor state (e.g., the target) with minimal magnitude of input uₜ. Concretely, in the finite horizon LQR model, the optimal control sequence (uₜ, t=0, 1, . . . . T−1) is computed by minimizing the following cost function:

$$J ( {u}_{0 : T - 1} ) = ( {\sum }_{t = 0}^{T - 1} ( {( {z}_{t} - {z}_{targ} )}^{T} Q ( {z}_{t} - {z}_{targ} ) + {u}_{t}^{T} {Ru}_{t} ) ) + {( {z}_{T} - {z}_{targ} )}^{T} {Q}_{T} ( {z}_{T} - {z}_{targ} )$$

In the model,

$$Q = 0 ∈ {R}^{( N + 1 + {N}_{c} ) \times ( N + 1 + {N}_{c} )} , R = I ∈ {R}^{N \times N} , and$$

$${Q}_{T} = [ \begin{matrix}0 ∈ {R}^{N \times N} & 0 & 0 \\ 0 & 0 ∈ {R}^{1} & 0 \\ 0 & 0 & I * {10}^{2} ∈ {R}^{{N}_{c} \times {N}_{c}}\end{matrix} ] ∈ {R}^{( N + 1 + {N}_{c} ) \times ( N + 1 + {N}_{c} )} .$$

Thus, the final cursor state error is penalized, and the magnitude of the input to the neural population uₜ is penalized (with setting R as non-zero). Because the magnitude of the input to neural activity is penalized, the controller sends the minimal input to the neural population to produce task behavior. The cost function is defined such that the cursor state during movement before the final cursor state is not penalized, and the neural state is never penalized.

The optimal control sequence (uₜ, t=0, 1, . . . . T−1) is given by uₜ=Kₜ (zₜ−z_{targ}) where feedback gain matrices (Kₜ^{lqr}, t=0, 1, . . . T−1) are computed iteratively solving the dynamic Ricatti equation backwards in time. The LQR solution for uₜ is computed using the dynamics of state error zₜ−z_{targ}, and that the dynamics of state error for non-zero target states are affine rather than strictly linear.

Center-out task simulations were run with the initial cursor position in the center of the workspace at c₀=[0, 0, 0, 0, 1] and the target cursor state at [targetₓ, target_{y}, velₓ=0, vel_{y}=0, 1]^{T}. Targets were positioned 10 cm away from the origin (same target arrangement as Monkey G). Target cursor velocity was set to zero to enforce that the cursor can stop at the desired target location.

Exact decoder parameters from Monkey G and linearized decoder parameters from Monkey J were used (F, K) in simulations. The invariant neural dynamics model parameters (A, b) were varied depending on the simulated experiment (see below). The horizon for each trial to hit its target state was set to be T=40 (corresponding to 4 seconds based on the BMI's timebin of 100 ms). Constraining each trial to be equal length facilitated comparison of performance across different simulation experiments.

In relation to OFC for obstacle-avoidance using a heuristic, Obstacle-avoidance task simulations were performed with the same initial and target cursor states as the center-out task, except that the cursor circumvented the obstacle to reach the target in both clockwise and counterclockwise movements. A heuristic strategy is used to direct cursor movements around the obstacle; a waypoint is defined as an intermediate state the cursor had to reach enroute to the final target. The heuristic solution performs optimal control from the start position to the waypoint, and then optimal control from the waypoint to the final target. Importantly, this solution minimizes the amount of input needed to accomplish these goals. A heuristic solution is used because the linear control problem of going from the initial cursor state to the final target cursor state with the constraint of avoiding an obstacle is not a convex optimization problem.

Concretely, for the first segment of the movement, a controller with a horizon T=20 directed the cursor to the waypoint, and then a controller with horizon T=20 directed the cursor from the waypoint to the final target (such that the trial length was matched to the center-out task simulation with T=40).

The waypoint was defined relative to the obstacle position as follows. First the vector between the center target and the obstacle position was determined (v_{obs,center}). The v_{obs,center} was then rotated either +90 degrees or −90 degrees corresponding to clockwise and counterclockwise movements. The waypoint position was a 6 cm distance in the direction of the rotated vector, from the obstacle center. Finally, the desired velocity vector of the intermediate target was set to be in the direction of v_{obs,center}, with a magnitude of 10 cm/s, so that the cursor would be moving in a direction consistent with reaching its final target in the second segment of the movement after the waypoint was reached.

To compute the input uₜ to execute these movements, the state error is defined at each time t as zₑᵣᵣₒᵣ=z_{targ}−zₜ, where z_{targ} was the waypoint for the first half of the movement, and z_{targ} was the final target for the second half of the movement. The linear quadratic regulator feedback gain Kₜ^{lqr} matrices were computed on the appropriate state error dynamics with the shortened horizon T=20.

Simulations of the “Full Dynamics Model” consisted of OFC with the invariant dynamics parameters (A, b) that were fit on experimentally-recorded neural activity from each subject and session (see “Invariant dynamics models” below, under “Quantification and Statistical Analysis”). Kₜ^{lqr} was computed using these experimentally-observed (A, b) parameters. The initial state of neural activity (e.g., xₜ at t=0) was set to the fixed point of the dynamics.

Simulations of the “No Dynamics Model” consisted of OFC with invariant dynamics parameter A set to zero (A=0). The experimentally-observed offset b was still used from each subject and session. Kₜ^{lqr} was computed using A=0 and the experimentally-observed b, and thus it was different than in the “Full Dynamics Model.” The initial state of neural activity (e.g., xₜ at t=0) was set to offset b, the fixed point of dynamics with A=0.

Simulations of the “Decoder-null Dynamics Model” consisted of OFC with the experimentally-observed invariant dynamics parameters (A, b) that were restricted to the decoder-null space, e.g., each invariant dynamics model was fit only on the projection of neural activity into the decoder-null space (see “Invariant dynamics models” under “Quantification and Statistical Analysis”). Kr was computed using these experimentally-observed decoder-null (A, b) parameters, and thus it was different than in the “Full Dynamics Model.” The initial state of neural activity (e.g., xₜ at t=0) was set to the fixed point of the decoder-null invariant dynamics.

The “Decoder-null Dynamics Model” was compared to its own “No Dynamics Model”, which consisted of OFC with Kₜ^{lqr} computed using A=0 and the experimentally-observed decoder-null offset b for each subject and session, and thus it was different than in the previously defined models. The initial state of neural activity (e.g., xₜ at t=0) was set to the decoder-null offset b, the fixed point of dynamics with A=0.

In the OFC model, movement errors arise due to noise in the neural activity, and subsequent neural activity issues commands based on feedback to correct these errors. Two considerations are used to choose the noise level for neural activity. First, a level of neural noise was added that was comparable to the neural “signal” needed to perform control in the absence of noise. Second, the same level of noise was added to the dynamics model (either “Full Dynamics Model” or “Decoder-null Dynamics Model”) and the corresponding “No Dynamics Model,” in order to facilitate comparison.

Thus, the “No Dynamics Model” is first simulated without noise for a single trial for each of 24 conditions, and a, the average variance of a neuron is calculated across time and trials.

Then for the noisy simulations of the “No Dynamics Model” and the corresponding dynamics models, Gaussian noise with zero mean and fixed variance a was added to each neuron at each timestep: xₜ=Axₜ₋₁+Buₜ₋₁+σₜ₋₁, where σₜ˜N(0, aI). Thus, the overall level of added noise (the sum of noise variance over neurons) matched the overall level of signal in the noiseless No Dynamics Model simulation (sum of activity variance over neurons).

Main findings as illustrated in FIGS. 16C-16D, 16G-16H apply with different noise levels.

In relation to command discretization for analysis, the occurrence of the same command is analyzed across different movements. Commands on individual time points were analyzed as the same command if they fell within the same discretized bin of continuous-valued, two-dimensional command space. All commands from rewarded trials in a given experimental session (including both tasks) were aggregated and discretized into 32 bins. Individual commands were assigned to one of 8 angular bins (bin edges were 22.5, 67.5, 112.5, 157.5, 202.5, 247.5, 292.5, and 337.5 degrees) and one of four magnitude bins. Angular bins were selected such that the straight line from the center to each of the center-out targets bisected each of the angular bins as has been done in previous work⁵⁰ (FIG. 17A). Magnitude bin edges were selected as the 23.75^{th}, 47.5^{th}, 71.25^{th}, and 95^{th} percentile of the distribution of command magnitudes for that experimental session. Commands falling between the 95^{th} and 100^{th} percentile of magnitude were not analyzed to reduce very infrequent noisy observations from skewing the bin edges for command magnitude.

Conditions that Used a Command Regularly

For each session, the number of times each of the 32 (discretized) commands was used in a given condition was tabulated. If the command was used >=15 times for that condition within a given session pooling across trials, that condition was counted as using the command regularly and was used in all analyses involving (command, condition) tuples. Commands that were used <15 times were not used in analysis involving (command, condition) tuples. The main results of the study were not affected by this particular selection. Typically, an individual command is used regularly in 5-10 conditions (distribution shown in FIG. 17A).

### Cursor and Command Trajectory Visualization

### Cursor Position Subtrajectories

To visualize the cursor position trajectories locally around the occurrence of a given command for each condition, the average position “subtrajectory” is computed, defined as the average trajectory in a window locked to the occurrence of the given command. For each condition, cursor positions from successful trials were aggregated. Cursor position subtrajectories shown in FIG. 11F are from representative session 0 from Monkey G. A matrix of x-axis and y-axis position trajectories was formed by extracting a window of −500 ms to 500 ms (5 previous samples plus 5 proceeding samples) around each occurrence of the given command in a given condition (total of N_{com-cond} occurrences, yielding a 2×11×N_{com-cond} matrix). Averaging over the N_{com-cond} observations yielded a condition-specific command-locked average position subtrajectory (size: 2×11) for each condition. If a command fell in the first 500 ms or last 500 ms of a trial, its occurrence was not included in the subtrajectory calculation. The position subtrajectories were translated such that the occurrence of the given command was set to (0, 0) in the 2D workspace (FIG. 11F right, FIG. 17C middle).

To visualize trajectories of commands around the occurrence of a given command for each condition (FIG. 11G, right), the same procedure applies as described above for cursor position subtrajectories to tabulate a 2×11×N_{com-cond} matrix but with x-axis and y-axis commands instead of positions. This matrix consisted of the continuous, two-dimensional velocity values of the commands. Averaging over the N_{com-cond} observations yielded the average condition-specific command subtrajectory (size: 2×11 array), as shown in FIG. 11F left for example conditions.

For matching the condition-pooled distribution, in many analyses, data (e.g. neural activity or a command-locked cursor trajectory) associated with a command and a specific condition is compared to data that pools across conditions for that same command (FIGS. 3-5). The distribution of the precise continuous value of the command within the command's bin can systematically differ between condition-specific and condition-pooled datasets. These differences are also referred to as “within-command-bin differences.” To ensure within-command-bin differences are not the source of significant differences between condition-specific and condition-pooled data associated with a command, a procedure is developed to sub-select observations of condition-pooled commands so that the mean of the condition-pooled command distribution is matched to the mean of the condition-specific command distribution. This procedure ensures that any differences between the condition-specific quantity and condition-pooled quantity are not due to ‘within-command-bin differences’. This procedure is performed on all analyses comparing condition-specific data to a condition-pooled distribution of data. The matching procedure is as follows:

From the condition-specific distribution, compute the command mean μ_{com-cond} (size: 2×1) and standard deviation σ_{com-cond} (size: 2×1).
1. Compute the deviation of each continuous-valued command observation in the condition-pooled distribution from the condition-specific distribution.
a. Use the condition-specific distribution's parameters to z-score the condition-pooled distribution's continuous-valued command observations by subtracting μ_{com-cond} and dividing by σ_{com-cond}.
b. Compute the deviation of condition-pooled observations from the condition-specific distribution as the L2-norm of the z-scored value
c. For indices in the condition-pooled distribution that correspond to data in the condition-specific distribution, over-write the L2-norm of the z-scored values with zeros. This step reduces the condition-pooled distribution from dropping datapoints that are in the condition-specific data, thereby ensuring the condition-pooled distribution contains the condition-specific data.
2. Remove the 5% of condition-pooled observations with the largest deviations
3. Use a Student's t-test to assess if the remaining observations in the condition-pooled distribution are significantly different than the condition-specific distribution for the first and second dimension of the command (two p-values)
4. If both p-values are >0.05, then the procedure is complete and the remaining observations in the condition-pooled distribution are considered the “command-matched condition-pooled distribution” for a specific command and condition.
5. If either or both p-values are <0.05, return to step 3 and repeat.

If the condition-pooled distribution cannot be matched to the condition-specific distribution such that the size of the condition-pooled distribution is larger than the condition-specific distribution, the particular (command, condition) will not be included in the analysis.

### Comparing Command Subtrajectories

To assess whether a command is used within significantly different command subtrajectories in different conditions (FIGS. 17D-17E), the following analysis is performed for conditions that have sufficient occurrences of the command (>=15):
1. The condition-specific average command subtrajectory is computed by averaging over N_{com-cond} single-trial command subtrajectories for the condition, as defined above in “Visualization of command subtrajectories”.
2. The condition-pooled average command subtrajectory is computed: all the single-trial command subtrajectories (N_{com}) are pooled across trials from all conditions that use the given command regularly (command occurs >=15 times in a session) to create a condition-pooled distribution of single-trial command subtrajectories (a 2×11× N_{com} matrix), which is then averaged to yield the condition-pooled average command subtrajectory (a 2×11 matrix).
3. In order to test whether condition-specific average command subtrajectories were significantly different from the condition-pooled average command subtrajectory, a distribution of subtrajectories was created by subsampling the condition-pooled distribution to assess expected variation in subtrajectories due to limited data. Specifically, N_{com-cond} single-trial command subtrajectories were sampled from a condition-pooled distribution of command subtrajectories that was command-matched to the specific condition (see above, “Matching the condition-pooled distribution”). These N_{com-cond} samples were then averaged to create a single subtrajectory, representing a plausible condition-specific average subtrajectory under the view that the condition-specific subtrajectories are just subsamples of the condition-pooled subtrajectories. This procedure was repeated 1000 times and used to construct a bootstrapped distribution of 1000 command subtrajectories.
4. This distribution was then used to test whether condition-specific subtrajectories deviated from the condition-pooled subtrajectory more than would be expected by subsampling and averaging the condition-pooled subtrajectory distribution. Specifically, the true condition-specific command subtrajectory distance from the condition-pooled command subtrajectory was computed (L2-norm between condition-specific 2×11 subtrajectory and condition-pooled 2×11 subtrajectory) and compared to the bootstrapped distribution of distances: (L2-norm between each of the 1000 subsampled averaged 2×11 command subtrajectories and the condition-pooled 2×11 command subtrajectory). A p-value for each condition-specific command subtrajectory distance was then derived.
The same analysis is also performed using only the next command following a given command (FIG. 17E).

Neural activity is shuffled in a manner that preserved behavior as a control for comparison against the hypothesis that neural activity follows invariant dynamics beyond the structure of behavior. Shuffled datasets preserved the timeseries of discretized commands but shuffled the neural activity that issues these commands. In order to create a shuffle for each animal on each session, all timebins from all trials from all conditions were collated. The continuous-valued command at each timebin was labeled with its discretized command bin. For each of the 32 discretized command bins, all timebins corresponding to a particular discretized command bin were identified. The neural activity in these identified timebins was then randomly permuted. A complete shuffled dataset was constructed by performing this random permutation for all discretized command bins. This full procedure was repeated 1000 times to yield 1000 shuffled datasets.

Activity issuing a given command is analyzed in relation to condition-specific neural activity distances.

For each session, (command, condition) tuples with >=15 observations were analyzed. For each of these (command, condition) tuples, the distance between condition-specific average activity and condition-pooled average activity is analyzed, both for individual neurons and for the population's activity vector (FIGS. 13B-13E).

### Analysis of Individual Neurons for a Given (Command, Condition) Tuple, Given N Neurons:

1. Compute the condition-specific average neural activity (μ_{com-cond}∈R^{N}) as the average neural activity over all observations of the command in the condition.
2. Compute the condition-pooled average activity (μ_{com-pool}∈R^{N}) as the average neural activity over observations of the command pooling across conditions. The command-matching procedure is used to form the condition-pooled dataset to account for within-command-bin differences (see “Matching the condition-pooled distribution” above).
3. Compute the absolute value of the difference between the condition-specific and condition-pooled averages: dμ_{com-cond}=abs(μ_{com-cond}−μ_{com-pool})∈R^{N}.
4. Repeat steps 1-3 for each shuffled dataset i, yielding dμ_{shuff-i-com-cond} for i=1:1000.
5. For each neuron j, compare dμ_{com-cond}(j) to the distribution of dμ_{shuff-i-com-cond}(j) for i=1:1000. Distances greater than the 95^{th} percentile of the shuffled distribution are deemed to have significantly different neuron j activity for a command-condition.

Analysis of population activity for a given (command, condition) tuple: To compute population distances, one extra step was performed. This can ensure that the distances calculated were not trivially due to “within-bin differences” between the condition-specific and condition-pooled distributions. The first step to ensure this was described above in “Matching the condition-pooled distribution”. The second step was to only compute distances in the dimensions of neural activity that are null to the decoder and do not affect the composition of the command. Thus, any subtle remaining differences in the distribution of commands would not influence population distances.

To compute distances in the dimensions of neural activity null to the decoder, an orthonormal basis of the null space of decoder matrix K∈R^{2×N} is computed using scipy.linalg.null_space, yielding Vₙᵤₗₗ∈R^{N×N-2}. The columns of V correspond to basis vectors spanning the N−2 dimensional null space. Using Vₙᵤₗₗ, it is computed that μ_{com-cond-null}=Vₙᵤₗₗ′*μ_{com-cond} and μ_{com-pool-null}=Vₙᵤₗₗ′*μ_{com-pool}. Then the population distance metric (L2-norm) is calculated, normalized by the square-root of the number of neurons:

$$d {μ}_{pop - com - cond} = / \sqrt[2]{N} , d {μ}_{pop - com - cond} ∈ {R}^{1} .$$

In step 5, the single value dμ_{pop-com-cond} is compared to the distribution of dμ_{shuff-i-pop-com-cond} for i=1:1000 to derive a p-value for each (command, condition) tuple. The fraction of (command, condition) tuples with population activity distances greater than the 95^{th} percentile of the shuffle data is reported in FIG. 13E.

For visualization of distances relative to the shuffle distribution (FIGS. 13B-13D), the observed population distance is divided for each (command, condition) tuple by the mean of the corresponding shuffle distribution. With this normalization, the spread of the shuffle distribution (FIG. 13B, right) can be visualized and a normalized distance of 1 can be interpreted as the expected distance according to the shuffle distribution.

In relation to activity distances pooling over conditions, to test whether condition-specific neural activity significantly deviated from condition-pooled neural activity for a given command (FIG. 13E, middle), the distance between condition-specific and condition-pooled average activity is aggregated over all Ncond conditions in which the command was used (>=15 occurrences of the command in a condition). An aggregate command distance is computed:

$$d {μ}_{pop - com} = \frac{1}{Ncond} {\sum }_{j = 1}^{Ncond} d {μ}_{pop - com - j} ,$$

and an aggregate shuffle distribution is computed:

$$d {μ}_{shuff - i - pop - com} = \frac{1}{Ncond} {\sum }_{j = 1}^{Ncond} d {μ}_{shuff - i - pop - com - j} .$$

Then, dμ_{pop-com} is compared to the distribution of dμ_{shuff-i-pop-com} for i=1:1000 to derive a p-value for each command. The fraction of commands with significant population activity distances is reported in FIG. 13E, middle.

In relation to single neuron distances, to test whether an individual neuron's condition-specific activity deviated from condition-pooled activity (FIG. 13E right), the distances between condition-specific and condition-pooled average activity is aggregated over the C (command, condition) tuples with at least 15 observations. The aggregated distance for neuron n was computed:

$$d μ ( n ) = \frac{1}{C} {\sum }_{c = 1}^{C} d {μ}_{c} ( n )$$

where dμ_{c}(n) is the condition-specific absolute difference for the nth neuron and cth (command, condition) tuple. Then dμ(n) was compared to the distribution of the aggregated shuffle:

$$d {μ}_{shuff - i} ( n ) = \frac{1}{C} {\sum }_{c = 1}^{C} d {μ}_{shuff - i - c} ( n )$$

for i=1:1000 to derive a p-value for each neuron. The fraction of neurons with significant activity distances (p-value<0.05) is reported in FIG. 13E right.

### Neural Activity Distances Summary

Single neuron activity distances reported in FIG. 18B (left) are for all (command, condition, neuron) tuples that had at least 15 observations. Distances are reported as a z-score of shuffle distribution:

$${z}_{c o m - c o n d} ( n ) = \frac{( d {μ}_{c o m - c ond} ( n ) - mean ( d {μ}_{s h u f f - i} , ( n ) i = 1 : 1000 ) )}{sfd ( d {μ}_{shuff - i} ( n ) , i = 1 : 1000 )} .$$

Single neuron activity distances reported in (FIG. 18B center, right) are for (command, condition, neuron) tuples that significantly deviated from shuffle. Raw distances in neuron activity are reported as dμ_{com-cond}(n) (FIG. 18B, center), and fraction distances as

$$\frac{d {μ}_{c o m - c o n d} ( n )}{{μ}_{c o m - p o o l} ( n )}$$

(FIG. 18B, right).

Population activity distances reported in FIGS. 13B-13D and FIG. 18C left are for all (command, condition) tuples. Distances in population activity are reported as a fraction of shuffle mean: dμ_{pop-com-cond}/mean (dμ_{shuff-i}, i=1:1000) (FIGS. 13B-13D), and as a z-score of shuffle distribution:

$${z}_{p o p - c o m - c o n d} = \frac{( {μ}_{p o p - c o m - c ond} - mean ( d {μ}_{shuff - i} , i = 1 : 1000 ) )}{sfd ( d {μ}_{shuff - i} , i = 1 : 1000 )}$$

(FIG. 18C left).

Population activity distances reported in FIG. 18C (center, right) are for (command, condition) tuples that significantly deviated from shuffle. Distances in population activity are reported as a fraction of shuffle mean dμ_{pop-com-cond}/mean (dμ_{shuff-i}, i=1:1000) (FIG. 18C, center) and fraction of condition-pooled activity as

$$\frac{d {μ}_{p o p - o u t - c o n d}}{{‖ {μ}_{c o m - p o o l} ‖}_{2}}$$

(FIG. 18C, right).

### Invariant Dynamics Models

In order to test whether invariant dynamics predicts the different neural activity patterns issuing the same command for different conditions, a linear model was fit for each experimental session on training data of neural activity from all conditions and assessed on held-out test data. Neural activity at time t, xₜ, was modeled as a linear function of xₜ₋₁:

$${x}_{t} = A {x}_{t - 1} + b$$

Here A∈R^{N×N} modeled invariant dynamics and b∈R^{N} was an offset vector that allowed the model to identify non-zero fixed points of neural dynamics. Ridge regression was used to estimate the A and b parameters. Prior to any training or testing, data was collated such that all neural activity in bins from t=2:Tₜᵣₗ in all rewarded trials were paired with neural activity from t=1: (Tₜᵣₗ−1), where Tₜᵣ is the number of time samples in a trial.

### Estimation of Ridge Parameter

For each experimental session, data collated from all conditions was randomly split into 5 sections, and a Ridge model (sklearn.linear_model.Ridge) with a ridge parameter varying from 2.5×10⁻⁵ to 10⁶ was trained using 4 of the 5 sections and tested on the remaining test section. Test sections were rotated, yielding five estimates of R² for each ridge parameter. The ridge parameter yielding the highest cross-validated mean R² was selected for each experimental session. Ridge regression was used primarily due to a subset of sessions with a very high number of units (148 and 151 units), thus a high number of parameters needed to be estimated for the A matrix. Without regularization, these parameters tended to extreme values, and the model generalized poorly.

### Invariant Dynamics Model: Fitting and Testing

Once a ridge parameter for a given experimental session was identified, A, b were again trained using 4/5 of the data. The remaining test data was predicted using the fit A, b. This procedure was repeated, rotating the training and testing data such that after five iterations, all data points in the experimental session had been in the test data section for one iteration of model-fitting. The predictions made on the held-out test data were collated together into a full dataset. Predictions were then analyzed in subsequent analyses.

### Generalization of Invariant Dynamics

It is assessed how well invariant dynamics generalized when certain categories of neural activity were not included in the training data. Invariant dynamics models were estimated after excluding neural activity in the following categories (FIG. 14C, FIGS. 20A-201, FIGS. 15C-15D):
1. Left-out Command: For each command (total of 32 command bins), training data sets were constructed leaving out neural activity that issued the command (FIG. 14C, FIGS. 20A-201, FIGS. 15C-15D).
2. Left-out Condition: For each condition (consisting of target, task, and clockwise or counterclockwise movement for obstacle avoidance), training data sets were constructed leaving out neural activity for the given condition (FIG. 14C, FIGS. 20A-201, FIGS. 15C-15D).
3. Left-out Command Angle: For each command angular bin (total of 8 angular bins), training data sets were constructed leaving out neural activity that issued commands in the given angular bin. This corresponds to leaving out neural activity for the 4 command bins that have the given angular bin but different magnitude bins (FIG. 20B, middle).
4. Left-out Command Magnitude: For each command magnitude bin (total of 4 magnitude bins), training data sets were constructed leaving out neural activity that issued commands of the given command magnitude. This corresponds to leaving out neural activity for the 8 command bins that have the given magnitude bin but different angle bins (FIG. 20B, right).
5. Left-out Classes of Conditions (FIG. 20G):
a. vertical condition class consisting of conditions with targets located at 90 and 270 degrees for both tasks,
b. horizontal condition class consisting of conditions with targets located at 0 and 180 degrees for both tasks,
c. diagonal 1 condition class consisting of conditions with targets located at 45 and 215 degrees for both tasks, and
d. diagonal 2 condition class consisting of conditions with targets located at 135 and 315 degrees for both tasks.

For each of the listed categories above, many dynamics models were computed—each one corresponding to the exclusion of one element of the category (e.g., one model per: command left-out, condition left-out, command angle left-out, command magnitude left-out, and class of conditions left-out). Each of the trained models was then used to predict the left-out data. Predictions were aggregated across all dynamics models resulting in a full dataset of predictions. The R² of this predicted dataset reflected how well dynamics models can generalize to types of neural activity that were not observed during training. Monkey J did not perform all conditions in the “diagonal 2” class, and so was not used in the analysis predicting excluded “diagonal 2” conditions.

As an additional comparison, invariant dynamics that lie only within the decoder-null space (the neural activity subspace that was orthogonal to the decoder such that variation of neural activity in this space has no effect on the decoder's output, e.g., commands for movement) are modeled as a Decoder-null dynamics model.

One approach was to project spiking activity into the decoder null space, and then fit invariant dynamics on the projected, decoder-null spiking activity. An orthonormal basis of the null space of decoder matrix K∈R^{2×N} is first computed using scipy.linalg.null_space, yielding Vₙᵤₗₗ∈R^{N×N-2}. The columns of V correspond to basis vectors spanning the N−2 dimensional null space. The projection matrix Pₙᵤₗₗ∈R^{N×N} is then computed where

$${P}_{n u l l} = {V}_{n u l l} {V}_{n u l l}^{T} .$$

Spiking activity was then projected into the null space

$${x}_{t}^{n u l l} = {P}_{n u l l} {x}_{t} , where {x}_{t}^{n u l l} ∈ {R}^{N \times 1} .$$

Following the above procedure (see “Estimation of Ridge Parameter”), a ridge regression parameter was selected using projected data xₜ^{null}. Decoder-null dynamics model parameters Aₙᵤₗₗ, bₙᵤₗₗ were then fit on ⅘ of the dataset and then tested on the remaining 1/5 of the

$${x}_{t}^{n u l l}$$

dataset. As verore, wie training/testing procedure was repeated 5 times such that all data points fell into the test dataset once. Predictions of test data from all five repetitions were collated into one full dataset of predictions.

### Shuffle Dynamics Model

The invariant dynamics model was compared to a shuffle dynamics model fit on shuffled data (see “Behavior-preserving shuffle of activity” above). Following the above procedure (see “Estimation of Ridge Parameter”), a ridge parameter was selected using shuffled data. Shuffle dynamics model parameters A_{shuffle}, b_{shuffle} were then fit on 4/5 of the dataset using shuffled data and then tested on the remaining 1/5 of the dataset using original, unshuffled data.

### Invariant Dynamics Model Characterization

### Dimensionality and Eigenvalues

Once the linear invariant dynamics model's parameters A, b were estimated, A was analyzed to assess which modes of dynamics¹⁶ were present (FIGS. 19A-19F). The eigenvalues of A were computed. From each eigenvalue, an oscillation frequency and time decay value were computed using the following equations:

$$Frequency = ∠λ / ( 2 πΔ t ) Hz if λ is complex , else frequency = 0 Hz$$

$$Time Decay = \frac{- 1}{ln ( \vert λ \vert )} Δ t sec$$

Modes of dynamics contributing substantially to predicting future neural variance will have time decays greater than the BMI decoder's binsize (here, 100 ms). 2-4 such dimensions of dynamics were found across sessions and subjects (FIGS. 19A-19F).

As illustrated in FIG. 15C, next activity xₜ₊₁ can be predicted based on current activity xₜ by taking the expected value according to the model: E(xₜ₊₁|xₜ, A, b)=Axₜ++b.

As illustrated in FIGS. 15D-15G, the next command commandₜ₊₁ can be predicted based on current neural activity xₜ by taking its expected value according to the model: E(commandₜ₊₁|xₜ, A, b, K)=K(Axₜ+b), where the decoder matrix K maps between neural activity and the command. This amounts to first predicting next activity based on current activity as above E(xₜ₊₁|xₜ, A, b)=Axₜ+b and then applying decoder K.

As illustrated in FIGS. 14C-14G, current activity xₜ can be predicted not only with knowledge of previous activity xₜ₋₁, but also with knowledge of the current command commandₜ (xₜ|xₜ₋₁, A, b, K, commandₜ). xₜ and xₜ₋₁ are modeled as jointly Gaussian with the dynamics model, and commandₜ is jointly Gaussian with them since commandₜ=Kxₜ. The prediction of xₜ is modified based on knowledge of commandₜ: E(xₜ|xₜ₋₁, A, b, K, commandₜ). Explicitly conditioning on commandₜ, it is thereby ensured that K*E(xₜ|xₜ₋₁, A, b, K, commandₜ)=commandₜ. To do this the joint distribution of xₜ and commandₜ is written as:

$$\begin{matrix}{x}_{t} \\ {Kx}_{t}\end{matrix} ∼ N ( ( \begin{matrix}μ \\ K μ\end{matrix} ) , ( \begin{matrix}\sum & {( K \sum )}^{T} \\ K \sum & K \sum {K}^{T}\end{matrix} ) )$$

where μ=E(xₜ|xₜ₋₁, A, b)=Axₜ₋₁+b, and Σ=cov[xₜ-(Axₜ₋₁+b)] is the covariance of the noise in the dynamics model. Then, the multivariate Gaussian conditional distribution provides the solution to conditioning on commandₜ:

E(xₜ|xₜ₋₁, A, b, K, commandₜ)=Axₜ₋₁+b+Σ^{T}K^{T}(KΣK^{T})⁻¹(commandₜ−K(Axₜ₋₁+b))

This prediction constrains the prediction of xₜ to produce the given command commandₜ.

For these predictions, E is estimated following dynamics model fitting and set to the empirical error covariance between estimates of E(xₜ)=Axₜ₋₁+b and true xₜ in the training data.

As illustrated in FIGS. 14C-14E, as a comparison to the dynamics prediction (xₜ|xₜ₋₁, A, b, K, commandₜ), xₜ is predicted as its expected value (xₜ|K, commandₜ) based only on the command commandₜ=Kxₜ it issues and the decoder matrix K. The same approach was used as above, except with empirical estimates of μ, Σ corresponding to the mean and covariance of the neural data instead of using the neural dynamics model and xₜ₋₁ to compute μ, Σ.

$$\begin{matrix}{x}_{t} \\ {Kx}_{t}\end{matrix} ∼ N ( ( \begin{matrix}μ \\ K μ\end{matrix} ) , ( \begin{matrix}\sum & {( K \sum )}^{T} \\ K \sum & K \sum {K}^{T}\end{matrix} ) )$$

This formulation makes the prediction:

$$E ( {x}_{t} \vert K , {command}_{t} ) = μ + {\sum }^{T} {{K}^{T} ( K \sum {K}^{T} )}^{- 1} ( {command}_{t} - K μ )$$

For the above predictions, it is evaluated whether invariant dynamics models were more accurate than shuffle dynamics. A distribution of shuffle dynamics R² values was generated by computing one R² value per shuffled dataset (see “Behavior-preserving shuffle of activity” above), where

$${R}_{s huffle , i , j}^{2}$$

corresponds to the R² for shuffle dataset i on session j. For each session j, each invariant dynamics model was considered significant if its R² was greater than 95% of shuffle R² values. To aggregate over S sessions, the R² values for all S sessions were averaged yielding one

$${R}_{a v g}^{2}$$

value. The average value was compared to a distribution of averaged shuffle R² values. Specifically, for each shuffle i (i=1:1000 shuffled dataset) an averaged R² value was computed across all S sessions:

$${R}_{avg , shuffle , i}^{2} = \frac{1}{S} {\sum }_{j = 1}^{S} {R}_{shuffle , i , j}^{2} ,$$

yielding a distribution of averaged shuffle R² values.

### Predicting Condition-Specific Activity

The invariant dynamics model was used to predict the condition-specific average activity for a given command (μ_{com-cond}, e.g., the average neural activity over all observations of the command in the condition, see “Analysis of activity issuing a given command” above) (FIGS. 14D-14G). The invariant dynamics model prediction ( [symbol] ) was computed as E(xₜ|xₜ₋₁, A, b, K, commandₜ) (see “Predicting activity issuing a given command” above) averaged over all observations of neural activity for the given command and condition.

To test if the invariant dynamics prediction was significantly more accurate than the shuffle dynamics model (e.g., the dynamics model fit on shuffled data, see “Shuffle dynamics model” above) prediction, the error is computed as the distance between true (μ_{com-cond}) and predicted ( [symbol] ) condition-specific average activity (single neuron error and population distance). Note that population distances for true and predicted activity were taken only in the dimensions null to the decoder (see “Condition-specific neural activity deviation”). The invariant dynamics model was deemed significantly more accurate than shuffle dynamics if the error was less than the 5^{th} percentile of the distribution of the errors from shuffle dynamics models. The fraction of (command, condition) tuples were individually significant relative to shuffle (FIG. 14G, left). It was determined that commands were individually significant relative to shuffle by analyzing the average population activity error across conditions (FIG. 4G, middle). It was determined that neurons were individually significant relative to shuffle by analyzing the average single-neuron error over (command, condition) tuples (FIG. 4G, right).

### Predicting Condition-Specific Component

The component of neural activity for a given command that was specific to a condition was calculated as μ_{com-cond}−E(x_{com-cond}^{t}|K, commandₜ), where μ_{com-cond} is neural activity averaged over observations for the given command and condition, and E(x_{com-cond}^{t}|K, commandₜ) is the prediction of neural activity only given the command it issued, averaged over observations for the (command, condition) tuple (see “Predicting current activity only with command” above). Thus, μ_{com-cond}−E(x_{com-cond}^{t}|K, commandₜ) estimates the portion of neural activity that cannot be explained by just knowing the command issued.

It is analyzed how well this condition-specific component can be predicted with invariant dynamics as: [symbol] −E(x_{com-cond}^{t}|K, commandₜ) (see “Predicting condition-specific activity” above for calculation of [symbol] ). The variance of μ_{com-cond}−E(x_{com-cond}^{t}|K, commandₜ) explained by [symbol] −E(x_{com-cond}^{t}|K, commandₜ) is reported in FIG. 14F.

### Predicting Condition-Specific Next Command

For each (command, condition) tuple, the average “next command” command_{com-cond} was calculated. For multiple observations of the given command in the given condition, the command at the time step was taken immediately following the given command and averaged over observations. Then it was analyzed how well invariant dynamics predicted this average “next command” [symbol] , calculated as E(commandₜ₊₁|xₜ, A, b, K) averaged over all observations of neural activity xₜ for the given command and condition. The L2-norm of the difference command_{com-cond}− [symbol] was computed and compared to the errors obtained from the shuffled-dynamics predictions. For each (command, condition) tuple, the dynamics-predicted “next command” was deemed significantly more accurate than shuffle dynamics if the error was less than the 5^{th} percentile of the distribution of the errors of the shuffled-dynamics predictions (FIG. 15E, left). Commands were determined to be individually significant if the error averaged over conditions was significantly less than the shuffled-dynamics error averaged over conditions (FIG. 15E, right).

In relation to analysis of predicted command angle, it can be further analyzed whether invariant dynamics predicted the transition from a given command to different “next commands” in different movements. Thus, two additional metrics are calculated on the direction of the predicted “next command”, e.g., the angle of the predicted “next command” [symbol] with respect to the condition-pooled “next command” command_{com-pool} (the average “next command” following a given command when pooling over conditions).

First, it can be predicted whether a condition's “next command” would rotate clockwise or counterclockwise relative to the condition-pooled “next command.” Specifically, it was calculated whether the sign of the cross-product between [symbol] and command_{com-pool} matched the sign of the cross-product between command_{com-cond} and command_{com-pool}. The fraction of (command, conditions) that were correctly predicted (clockwise vs counterclockwise) was compared to the fraction of (command, condition) tuples correctly predicted in the shuffle distribution (FIG. 15G, left).

Second, the absolute error of the angle between the predicted “next command” and the condition-pooled “next command” is calculated for each (command, condition) tuple:

abs(Ð( [symbol] , command_{com-pool})−(command_{com-cond}, command_{com-pool})

Explicitly, for each (command, condition) tuple, the absolute difference is calculated between two angles: 1) the angle between the predicted “next command” and the condition-pooled “next command” and 2) the angle between the true “next command” and the condition-pooled “next command”. These errors were then compared to the shuffle distribution (FIG. 15G, right).

### Estimation of Behavior-Encoding Models

To compare invariant dynamics models to models in which neural activity encodes behavioral variables in addition to the command, a series of behavior-encoding models (FIGS. 21A-21F) is fitted. Regressors included cursor state (position, velocity), target position (x,y postion in cursor workspace), and a categorical variable encoding target number (0-7) and task (“center-out”, “clockwise obstacle-avoidance”, or “counter-clockwise obstacle-avoidance”).

Models were fit using Ridge regression following the same procedure described above (see “Estimation of Ridge Parameter”) was followed with one additional step: prior to estimating the ridge parameter or fitting the regression, variables were z-scored. Without z-scoring, ridge regression can favor giving explanatory power to the variables with larger variances, since they can have smaller weights which work well with ridge regression. Then, as above, models were fit using ⅘ of the data and then used to predict the held-out ⅕ of data. After 5 rotations of training and testing data, a full predicted dataset was collated.

It is then tested whether invariant neural dynamics improved the prediction of neural activity beyond behavior-encoding. The R² of the model containing all regressors except previous neural activity was compared to the R² of the model containing all regressors plus previous neural activity (FIG. 21B) using a paired Student's t-test where session was paired. One test was done for each monkey.

For analysis between pairs of conditions, it is assessed whether the invariant dynamics model predicted the relationship between pairs of conditions for neural activity and behavior (FIGS. 22A-22K).

In relation to average neural activity for a given command, the invariant dynamics model was used to predict the distance between average neural activity patterns for the same command across pairs of conditions. Concretely, the predicted distance was simply the distance between the predicted neural activity pattern for condition 1 and for condition 2. The correlation between the true distance and the predicted distance was reported for individual neurons (FIGS. 22A-22C) and population activity (FIGS. 22B, 22D). The Wald test (implemented in scipy.stats.linregress) was used to assess the significance of the correlations on single sessions. To assess significance pooled over sessions, data points (true distances vs. dynamics model predicted distances) were aggregated across sessions and assessed for significance.

### Average Next Command

The invariant dynamics model was used to predict the distance between “next commands” for the same given command across pairs of conditions. Concretely, the predicted distance was simply the distance between the predicted “next command” for condition 1 and for condition 2. The correlation between the true distance and the predicted distance was reported (FIGS. 22J, 22K). As above, the Wald test was used to assess significance of correlations on single sessions and over pooled sessions.

For correlating neural distance with behavior, it can be determined whether neural activity for a given command was more similar across conditions with more similar command subtrajectories (see “Command subtrajectories”) (FIG. 22E), and whether invariant dynamics predict this. Specifically, the correlation is analyzed from the distance between average neural activity across two conditions for a given command to the distance between command subtrajectories for the same two conditions (FIGS. 22F top, 22G-22H left). Further, invariant dynamics can predict this correlation (FIGS. 22F bottom, 22G-22H right). For a command (that was used in more than five conditions) and pair of conditions that used the command (>=15 observations in each condition in the pair), 1) the distances between condition-specific average activity were computed and 2) distances between command subtrajectories were computed. The neural activity distances were correlated with the command subtrajectory distances (FIGS. 22F top, 22G-22H left). To assess whether invariant dynamics made predictions that maintained this structure, that same analysis is performed with distances between dynamics-predicted condition-specific average activity across pairs of conditions (FIGS. 22F bottom, 22G-22H right).

The significance of the relationship is assessed using a linear mixed effects (LME) model (statsmodels.formula.api.mixedlm). The LME modeled command as a random effect because the exact parameters of the increasing linear relationship between command subtrajectories and population activity can vary depending on command. Individual sessions were assessed for significance. To assess significance across sessions, data points were aggregated over sessions, and the LME model used command and session ID as random effects.

In relation to analysis of Optimal Feedback Control Models, for each simulated trial, the magnitude of input to the neural population is computed as the L2 norm of the input matrix uₜ∈R^{N×T} (where N is the number of neurons and T=40 was the horizon and thus movement length). For each of the 24 conditions, the average input magnitude is calculated over the 20 trials. The magnitude of input used by the Invariant Dynamics Model is compared to the No Dynamics Model, where the Invariant Dynamics Model was either the Full Dynamics Model (FIG. 16C) or the Decoder-Null Dynamics Model (FIG. 16D). Each individual session is analyzed with a paired Wilcoxon signed-rank test, where each pair within a session consisted of one condition (24 conditions total). These results are aggregated across sessions for each subject using a linear mixed effect (LME) model between input magnitude and model category (Invariant Dynamics Model or No Dynamics Model), with session modeled as a random effect.

Simulated activity can issue a given command. In the OFC simulations, different neural activity patterns can be used to issue the same command across different conditions, applying analyses used on experimental neural data to the OFC simulations. As above, discretized command bins (see “Command discretization for analysis”) are defined and calculated for the average neural activity for each (command, condition) tuple. For (command, condition) tuples with >=15 observations (example shown in FIG. 16E), the distance is computed between condition-specific average activity and condition-pooled average activity by subtracting the activity, projecting into the decoder-null space, taking the L2 norm, and normalizing by the square root of the number of neurons, as in the experimental data analysis (see “Analysis of activity issuing a given command”).

The distance between condition-specific average activity and condition-pooled average activity for a given command is analyzed, comparing each model to its own shuffle distribution (see “Behavior-preserving shuffle of activity”) (FIGS. 16G-16H). Concretely, for each simulated session, the mean of the shuffle distribution of distances for each (command, condition) tuple is calculated and these shuffle means (one per (command, condition) tuple) are compared to the observed distances from the simulations. Individual sessions are analyzed with a Mann-Whitney U test. These results are aggregated across sessions for each subject with a LME model between activity distance and data source (OFC Simulation vs shuffle), with session modeled as a random effect. For visualization of distances relative to the shuffle distribution (FIG. 16F-H), the observed distance for each (command, condition) tuple is divided by the mean of the corresponding shuffle distribution (same as in FIGS. 13B-13D).

For statistics Summary, in many analyses, it can be assessed whether a quantity calculated for a specific condition was significantly larger than expected from the distribution of the quantity due to subsampling the condition-pooled distribution. A p-value was computed by comparing the condition-specific quantity to the distribution of the quantity computed from subsampling the condition-pooled distribution. The “behavior-preserving shuffle of activity” and “matching the condition-pooled distribution” (see above) were used to construct the condition-pooled distribution.

The following is a summary of these analyses:
FIG. 17D, Quantity: distance between condition-specific average command subtrajectory and condition-pooled average command subtrajectory, P-value: computed using behavior-preserving shuffle.
FIG. 17E, Quantity: distance between condition-specific average next command and the condition-pooled average next command, P-value: computed using behavior-preserving shuffle.
FIG. 13B left, 3E right: Quantity: for a given command, distance between condition-specific average activity for a neuron and condition-pooled average activity for a neuron, P-value: behavior-preserving shuffle.
FIG. 13B right, 3D, 3E left, middle: Quantity: for a given command, distance between condition-specific average population activity and condition-pooled average population activity, P-value: behavior-preserving shuffle.
FIG. 14G right: Quantity: for a given command, error between the invariant dynamics' prediction of condition-specific average activity for a neuron and the true condition-specific average activity for the neuron. P-value: distribution of prediction errors from shuffle dynamics (models fit on behavior-preserving shuffle and that made predictions using unshuffled data).
FIG. 14G left, middle: Quantity: for a given command, error between the invariant dynamics' prediction of condition-specific average population activity and the true condition-specific average population activity. P-value: distribution of prediction errors from shuffle dynamics (models fit on behavior-preserving shuffle and that made predictions using unshuffled data).
FIG. 15E: Quantity: for a given command, error between the invariant dynamics' prediction of condition-specific average next command and true condition-specific average next command. P-value: distribution of prediction errors from shuffle dynamics (models fit on behavior-preserving shuffle and that made predictions using unshuffled data).

In the above analyses, the fraction of condition-specific quantities is also assessed which were significantly different from the condition-pooled quantities or significantly predicted compared to a shuffled distribution (FIGS. 17D-17E, FIG. 13E, FIG. 14G, FIG. 15E, FIGS. 20D-20I, FIG. 22G). In order to aggregate over all data to determine whether condition-specific quantities were significantly different from shuffle or significantly predicted within a session relative to shuffle dynamics, the condition-specific quantity is averaged over the relevant dimensions (command, condition, and/or neuron) to yield a single aggregated value for a session. For example in FIG. 13E right, the distance between average activity for a (command, condition, neuron) tuple and the condition-pooled average activity for a (command, neuron) tuple are averaged over (command, condition) tuples to yield an aggregated value that is used to assess if individual neurons are significant. The shuffle distribution is correspondingly averaged across all relevant dimensions (command, condition, and/or neuron). Together this procedure yielded a single aggregated value that can be compared to a single aggregated distribution to determine session significance. Finally, for aggregating over sessions, the condition-specific quantity can be taken which was aggregated within a session and averaged it across sessions and again compared it to a shuffle distribution of this value aggregated over sessions.

When R² was the metric assessed (FIGS. 14C-14F, FIGS. 15C-15D, FIGS. 20B, 20F, 20G), a single R² metric was computed for each session and compared to the R² distribution from shuffle models.

In some cases, a linear regression was fit between two quantities (FIGS. 22C, 22D, 22G, 22J, 22K) on both individual sessions and on data pooled over all sessions, and the significance of the fit and correlation coefficient were both reported. In other cases where random effects such as session or analyzed command can have influenced the linear regression parameters (FIGS. 22F-22G), a Linear Mixed Effect (LME) model was used with session and/or command modeled as random effects on intercept.

In FIGS. 21A-21F, a paired Student's t-test was used to compare two models' R² metric across sessions. FIG. 16 analyzed simulations of OFC models, not experimentally-recorded data. FIGS. 16C-16D used a paired Wilcoxon test and a LME to compare input magnitude between a pair of OFC models. FIGS. 16G-16H used a Mann-Whitney U test and a LME to compare population distance between an OFC model and its shuffle distribution.

What is claimed:

1. A method of modeling and decoding neural activities, comprising:
measuring, by a brain-machine interface (BMI), activities of a neural population;
receiving, by a processor, a first plurality of signals corresponding to the activities of the neural population from the BMI;
generating, by the processor applying a neural dynamics model to the first plurality of signals, a second plurality of signals corresponding to a de-noised state of the activities of the neural population;
generating, by the processor applying a BMI decoding model to the second plurality of signals, a control signal;
generating, by the processor applying a BMI plant model to the control signal, a movement vector;
moving an object according to a predetermined path, by the processor, in response to the movement vector and a current state of the object; and
in response to the moving of the object, continuously measuring, by the BMI, the activities of the neural population,
wherein a first plurality of coefficients of the neural dynamics model and a second plurality of coefficients of the BMI decoding model are iteratively updated, by the processor executing a learning algorithm, in response to the BMI's continuous measurement of the activities of the neural population.

2. The method of claim 1, further comprising:
constructing, by the processor, an input inference model configured to update a first state of the activities of the neural population to a second state of the activities of the neural population in response to an input signal;
moving the object, by the processor, according to the movement vector and in response to a perturbance vector generated by the processor;
measuring, by the BMI, an error signal corresponding to a change of the activities of the neural population based on the object moving in response to the perturbance vector; and
updating, by the processor executing a calibration algorithm, a plurality of coefficients of the input inference model,
wherein the BMI decoding model, when applied by the processor, is configured to process the first plurality of signals by combining the neural dynamics model and the input inference model.

3. The method of claim 1, wherein the object's motion is defined by a vector including a first component corresponding to a position and a second component corresponding to a velocity.

4. The method of claim 3, wherein the first component and the second component of the vector are linearly updated by a command vector.

5. The method of claim 1, wherein the object is a physical prosthetic device.

6. The method of claim 1, wherein the object is a cursor on a display.

7. The method of claim 1, wherein the neural dynamics model includes at least one invariant parameter across various neural activities.

8. The method of claim 1, further comprising:
constructing, by the processor, an augmented dynamics model; and
generating, by the processor applying the augmented dynamics model, an augmented dynamics signal,
wherein the BMI plant model, when applied by the processor, is configured to generate the movement vector in response to the augmented dynamics signal and the control signal generated by the BMI decoding model.

9. The method of claim 8, wherein the augmented dynamics model includes at least one invariant parameter across various neural activities.

10. The method of claim 1, wherein the neural dynamics model comprises:
a neural state model configured to output a neural state signal, the neural state signal corresponding to a recurrent dynamic of the neural population; and
a neural input model configured to output a neural input signal.

11. A system comprising:
a computing device, the computing device comprising a processor and a memory device in communication with the processor; and
a brain sensing device configured to measure activities of a neural population and send signals corresponding to the measured activities to the computing device,
wherein the processor, when executing instructions stored in the memory device, is configured to:
receive a first plurality of signals corresponding to the activities of the neural population from the brain sensing device;
generate, by applying a neural dynamics model to the first plurality of signals, a second plurality of signals corresponding to a de-noised state of the activities of the neural population;
generate, by applying a BMI decoding model to the second plurality of signals, a control signal;
generate, by applying a BMI plant model to the control signal, a movement vector; and
move an object according to a predetermined path, in response to the movement vector and a current state of the object,
wherein a first plurality of coefficients of the neural dynamics model and a second plurality of coefficients of the BMI decoding model are iteratively updated, by the processor executing a learning algorithm, in response to the brain sensing device's continuous measurement of the activities of the neural population.

12. The system of claim 11, the processor further configured to:
construct an input inference model configured to update a first state of the activities of the neural population to a second state of the activities of the neural population in response to an input signal;
move the object according to the movement vector and in response to a perturbance vector generated by the processor; and
update, by executing a calibration algorithm, a plurality of coefficients of the input inference model,
wherein the BMI decoding model, when applied by the processor, is configured to process the first plurality of signals by combining the neural dynamics model and the input inference model.

13. The system of claim 11, wherein the object's motion is defined by a vector including a first component corresponding to a position and a second component corresponding to a velocity.

14. The system of claim 13, wherein the first component and the second component of the vector are linearly updated by a command vector.

15. The system of claim 11, wherein the object is a physical prosthetic device.

16. The system of claim 11, wherein the object is a cursor on a display.

17. The system of claim 11, wherein the neural dynamics model includes at least one invariant parameter across various neural activities.

18. The system of claim 11, the processor further configured to:
construct an augmented dynamics model; and
generate, by applying the augmented dynamics model, an augmented dynamics signal,
wherein the BMI plant model, when applied by the processor, is configured to generate the movement vector in response to the augmented dynamics signal and the control signal generated by the BMI decoding model.

19. The system of claim 18, wherein the augmented dynamics model includes at least one invariant parameter across various neural activities.

20. The system of claim 11, wherein the neural dynamics model comprises:
a neural state model configured to output a neural state signal, the neural state signal corresponding to a recurrent dynamic of the neural population; and
a neural input model configured to output a neural input signal.

21. A method of simulating neural activities, comprising:
generating, by a processor applying a feedback control model, an input signal;
generating, by a processor applying a simulated neural dynamics model, a state signal;
combining, by a neural activity simulation program, the input signal and the state signal;
in response to combining the input signal and the state signal, generating a simulated neural activity signal; and
sending the simulated neural activity signal to a BMI system, the BMI system configured to be controlled by the neural activity simulation program.

22. The method of claim 21, further comprising:
measuring, by a device, a movement signal, the movement signal corresponding to a subject's physical movement; and
transforming, by the processor, the movement signal to a command signal,
wherein generating the simulated neural activity signal comprises inferring by the processor the simulated neural activity signal from the transformed command signal.
